(19) United States
(12) Patent Application Publication
(10) Pub. No.: US 2025/0290037 A1
(43) Pub. Date: Sep. 18, 2025

(54) PATHOLOGY-RESPONSIVE RECOMBINANT CELLS AND USES THEREOF

(71) Applicant: THE REGENTS OF THE UNIVERSITY OF CALIFORNIA, Oakland, CA (US)

(72) Inventors: Mathew Blurton-Jones, Irvine, CA (US); Jean Paul Chadarevian, Irvine, CA (US); Robert Spitale, Irvine, CA (US); Sunil Gandhi, Irvine, CA (US); Hayk Davtyan, Irvine, CA (US); Jonathan Hasselmann, Irvine, CA (US)

(21) Appl. No.: 18/860,346

(22) PCT Filed: Apr. 27, 2023
(86) PCT No.: PCT/US23/66323

§ 371 Date: Oct. 25, 2024

Related U.S. Application Data
(60) Provisional application No. 63/335,585, filed on Apr. 27, 2022.

(51) Int. Cl. C12N 5/079; A61K 35/15; A61K 35/28; A61K 35/30; A61P 25/28; C12N 5/0786; C12N 5/0789; C12N 5/0797; G01N 33/68

(52) U.S. Cl. CPC C12N 5/0622; A61K 35/15; A61K 35/28; A61K 35/30; A61P 25/28; C12N 5/0623; C12N 5/0645; C12N 5/0647; G01N 33/6896; C12N 2510/02

(57) ABSTRACT

Modified cells that express and present or secrete at least one therapeutic molecule that can treat or ameliorate a disease of interest such as but not limited to Alzheimer's disease. In the modified cells, expression of the therapeutic molecule is induced when the modified cells are proximate to or in contact with pathology related to the disease of interest. The present disclosure also relates to compositions and kits comprising the disclosed cells. The present disclosure also relates to methods of using the disclosed cells for treating disease.

### CROSS-REFERENCES TO RELATED APPLICATIONS

This application claims benefit of U.S. Provisional Application No. 63/335,585 filed Apr. 27, 2022, the specification of which is incorporated herein in their entirety by reference.

### STATEMENT REGARDING FEDERALLY SPONSORED RESEARCH OR DEVELOPMENT

This invention was made with government support under Grant No.DA048813 and Grant No. AG073787 awarded by National Institutes of Health. The government has certain rights in the invention.

### REFERENCE TO A SEQUENCE LISTING

The contents of the electronic sequence listing (name of the file UCI_22_14_PCT.xml; Size: 27,817 bytes; and Date of Creation: Apr. 26, 2023) is herein incorporated by reference in its entirety.

### FIELD OF THE INVENTION

The present invention relates to cells that have been modified to express at least one therapeutic molecule that can treat or ameliorate pathologies and/or symptoms related to particular conditions, such as Alzheimer's disease. The present invention may also relate to compositions and kits comprising the aforementioned cells as well as methods of use thereof for treating Alzheimer's disease.

### BACKGROUND OF THE INVENTION

Several prevalent neurodegenerative diseases are characterized by the progressive accumulation of insoluble proteinaceous aggregates including Alzheimer's Disease, Parkinson's Disease, Huntington's Disease, and amyotrophic lateral sclerosis (ALS). In Alzheimer's Disease, amyloid beta (Aβ) adopts varying conformational states including insoluble fibrillar Aβ plaques and soluble Aβ oligomers which can drive the development of additional downstream pathologies including neurofibrillary tangles and gliosis. Together these pathologies lead to neuronal and synaptic loss and brain atrophy, resulting in memory loss, cognitive impairment, behavioral changes, and dementia. Approximately 6 million people in the United States over the age of 65 are currently living with Alzheimer's disease. Alzheimer's disease is primarily a condition of later life. Thus, as populations age, the incidence of this disease is expected to grow. Thus, new therapeutics must be developed to address this increased need. However, one challenge to developing drugs for treating brain disorders is the blood-brain barrier (BBB), which presents a significant hurdle to the delivery, and, therefore, the efficacy of drugs. Both small molecules and macromolecules have been investigated as effective therapeutic agents to treat brain diseases. However, due to physical constraints on molecules capable of crossing the BBB, most macromolecules cannot penetrate the brain endothelium. Thus, despite decades of work to develop safe and effective therapies for brain disease, delivery of medicines across the BBB at adequate concentrations for target engagement remains a fundamental obstacle.

In recent years, CAR T-cell therapy has demonstrated the power of employing engineered cells to deliver therapeutic agents directly and selectively to the site of cancer pathology. For central nervous system disorders, neural and bone marrow stem cells have been explored as vehicles for delivering therapeutic agents. Unfortunately, human neural stem cells have worrying tumorigenic potential, and bone marrow stem cells require dangerous preconditioning steps to deliver cells into the brain. Therefore, a safe cellular delivery vehicle for the selective delivery of therapies directly to sites of CNS pathologies is still critically needed.

Microglia are the primary innate immune cells of the brain and play a critical role in maintaining neuronal homeostasis and surveying their local environment for pathogenic agents and neuronal damage. Recently, a chimeric mouse model was developed that allows examination of the interactions between human iPSC-derived microglia and neuropathology. In this model, after xenotransplantation, human iPSC-derived microglia (xMGs) disperse widely, mature into homeostatic microglia that exhibit in vivo-like transcriptional profiles and thrive. The present disclosure describes how such cells may be modified for the treatment of Alzheimer's disease and other Aβ-related neurodegenerative disorders.

### BRIEF SUMMARY OF THE INVENTION

It is an objective of the present invention to provide compositions and methods that allow for treating or preventing diseases or conditions associated with amyloid beta-related pathologies, including but not limited to Alzheimer's Disease (AD), or ameliorating or delaying symptoms and/or pathological processes associated with the amyloid beta-related pathology. In some embodiments, the amyloid beta-related pathology is associated with Alzheimer's disease. In some embodiments, the amyloid beta-related pathology is associated with Parkinson's disease. In some embodiments, the amyloid beta-related pathology is associated with Huntington's disease. In some embodiments, the amyloid beta-related pathology is associated with amyotrophic lateral sclerosis (ALS). The present invention is not limited to the aforementioned diseases or conditions associated with amyloid beta pathology.

Embodiments of the present invention can be freely combined with each other if they are not mutually exclusive.

Whereas immune-deficient mice that harbor deletions in Rag2 and il2 receptor gamma (il2rγ) genes have previously been used to transplant human cells, initial attempts to generate human microglial chimeric mice using standard immune-deficient mice failed. It was surprisingly discovered after multiple crosses and experiments that a mouse model harboring humanized CSF-1 alleles was necessary to enable long-term engraftment and survival of human xenotransplanted microglia (Hasselmann et. al., Neuron, 2019; Supp Fig S2). Not only was the generation of this model a challenge, but additional difficulties included backcrossing the model onto an amyloid-producing line (5×FAD mice) and restoring homozygosity for the Rag2, il2rγ humanized CSF-1 alleles.

Murine microglia have been shown to be highly sensitive to isolation procedures (Marsh et. al., Nat Neurosci. 2022). Despite the challenges, Inventors were able to develop a rapid approach to purify engrafted human microglia with minimal disruption of engrafted human microglia. This was achieved using a negative magnetic sorting approach to deplete all mouse cells, leaving behind untouched but highly pure and viable human microglia. The details and validation of this novel isolation approach are provided in Hasselmann et. al., Neuron, 2019. The approach to isolation allowed for the isolation of human microglia from the brains of chimeric mice to examine gene expression and identify candidate microglial genes that exhibit changes in expression in response to beta-amyloid pathology.

The above isolation approach enabled single cell sequencing which provided a partial list of plaque-responsive microglia genes. However, single cell sequencing is less sensitive than bulk RNA sequencing and typically only captures the most abundantly expressed transcripts. Despite the challenges, Inventors were able to develop an approach to isolate plaque-responsive versus non-plaque-responsive microglia from the same chimeric mouse brains, which allowed for a better understanding of the response of human microglia to plaques. The single cell sequencing data and subsequent immunohistochemical validation (see FIGS. 1A-1P) demonstrated that CD9 and HLA-DRB are highly enriched in plaque-associated human microglia. Inventors also developed a fluorescent-activated cell sorting (FACS) approach to isolate CD9/HLA-DRB double positive microglia versus double negative homeostatic microglia. This was achieved using mice transplanted with four independent human microglia samples and then performing bulk RNA sequencing. This analysis provided the more complete dataset of plaque-induced human microglia genes as described herein (see FIGS. 20A-20D).

One aspect of this disclosure provides a modified cell for treating amyloid beta-related pathology (e.g., Alzheimer's disease (AD), Parkinson's disease, Huntington's disease, ALS, other neurodegenerative disorder, e.g., other Aβ-related neurodegenerative disorders) or ameliorating symptoms and/or pathological processes associated with said amyloid beta-related pathology. The cell is sensitive to amyloid beta(Aβ)-related pathology. For example, in some embodiments, the modified cell expresses, presents, secretes, or a combination thereof a therapeutic molecule when the modified cell is proximal to or in contact with Aβ-related pathology (which may also be referred to herein as Alzheimer's Disease-related pathology), e.g., β-amyloid (Aβ) peptide plaques, soluble Aβ monomers, insoluble Aβ monomers, Aβ oligomers, pyroglutamate Aβ, protofibrils, fibrils comprising Aβ of varying lengths, or a combination thereof. In some embodiments, the modified cell expresses and secretes a therapeutic molecule when the modified cell is proximal to or in contact with an amyloid beta-related pathology (e.g., Alzheimer's disease-related pathology). In some embodiments, the modified cell expresses, presents, and secretes a therapeutic molecule when the modified cell is proximal to or in contact with an amyloid beta-related pathology. The therapeutic molecule alters one or more amyloid beta-related pathology phenotypes or at least one aspect of the amyloid beta-related pathology. For example, in some embodiments, the therapeutic molecule reduces the size and/or number of Aβ aggregates. The present invention is not limited to the aforementioned example of altering an amyloid beta-related pathology phenotype.

The amyloid beta-related pathology phenotype or symptom may comprise one or a combination of memory problems, learning deficits, cognitive problems, vision problems, behavioral changes, personality changes, depression, or seizures. In some embodiments, the Aβ-related pathology may comprise β-amyloid (Aβ) peptide plaques. In some embodiments, the Aβ-related pathology may comprise soluble Aβ monomers. In some embodiments, the Aβ-related pathology may comprise insoluble Aβ monomers. In some embodiments, the Aβ-related pathology may comprise Aβ oligomers. In some embodiments, the Aβ-related pathology may comprise protofibrils. In some embodiments, the Aβ-related pathology may comprise fibrils comprising Aβ of varying lengths. In some embodiments, the Aβ-related pathology may comprise one or a combination of: β-amyloid (Aβ) peptide plaques, soluble Aβ monomers, insoluble Aβ monomers, Aβ oligomers, pyroglutamate Aβ, protofibrils, fibrils comprising Aβ of varying lengths, or a combination thereof.

The modified cell may comprise a nucleic acid sequence encoding the therapeutic molecule. The nucleic acid sequence may be operatively linked to a pathology-responsive promoter, e.g., a promoter responsive to pathology associated with Alzheimer's disease, Parkinson's disease, Huntington's disease, ALS, etc., e.g., an amyloid beta-responsive promoter. As a non-limiting example, the nucleic acid may be operatively linked to a promoter responsive to Aβ peptides, e.g., the promoter may be activated when the cell is in proximity to or in contact with Aβ peptides. In some embodiments, the therapeutic molecule may cleave Aβ peptides. In some embodiments, the therapeutic molecule may reduce the amount of Aβ peptide in Aβ peptide plaques in an individual's brain. In some embodiments, the therapeutic molecule may reduce the size or number of soluble Aβ monomers, insoluble Aβ monomers, Aβ oligomers, pyroglutamate Aβ, protofibrils, or fibrils comprising Aβ of varying lengths. In some embodiments, the therapeutic molecule may reduce the size and/or number of Aβ aggregates. In some embodiments, the therapeutic molecule may enhance amyloid proteolysis. In some embodiments, the therapeutic molecule may enhance microglial phagocytosis of amyloid beta. The present invention is not limited to the aforementioned mechanisms of action for altering an amyloid beta-related pathology phenotype.

One aspect is a modified cell comprising a nucleic acid sequence encoding a therapeutic molecule, wherein the nucleic acid sequence is operatively linked to a promoter that activates transcription of the therapeutic molecule when the cell is proximal to or in contact with, β-amyloid (Aβ) peptide plaques, soluble Aβ monomers, insoluble Aβ monomers, Aβ oligomers, pyroglutamate Aβ, protofibrils, or fibrils comprising Aβ of varying lengths. In some embodiments, the therapeutic molecule cleaves Aβ peptide. In some embodiments, the therapeutic molecule reduces the amount of Aβ peptide in Aβ peptide plaques in an individual's brain. In some embodiments, the therapeutic molecule reduces the size or number of soluble Aβ monomers, insoluble Aβ monomers, Aβ oligomers, pyroglutamate Aβ, protofibrils, or fibrils comprising Aβ of varying lengths. In some embodiments, the therapeutic molecule reduces the size and/or number of Aβ aggregates. In some embodiments, the therapeutic molecule enhances amyloid proteolysis. In some embodiments, the therapeutic molecule enhances microglial phagocytosis of amyloid beta.

In some embodiments, the therapeutic molecule is a therapeutic mRNA molecule. In some embodiments, the therapeutic molecule is a therapeutic protein. The therapeutic protein may be a membrane-bound protein or a secreted protein. Secreted therapeutic proteins may include modified proteins. For example, in some embodiments, the therapeutic protein is a protein having been modified to lack a cytoplasmic domain. In some embodiments, the therapeutic protein is a protein having been modified to lack a transmembrane domain. In other embodiments, the therapeutic protein is a protein having been modified to lack a transmembrane domain and a cytoplasmic domain. In some embodiments, the therapeutic molecule comprises an enzyme or an immune-modulating protein. In some embodiments, the therapeutic molecule comprises TREM2. In some embodiments, the therapeutic molecule comprises an insulin degrading enzyme. In some embodiments, the therapeutic molecule comprises MSR1 (SCARA1). In some embodiments, the therapeutic molecule comprises LRP1. In some embodiments, the therapeutic molecule comprises APOE. In some embodiments, the therapeutic molecule comprises IL4. In some embodiments, the therapeutic molecule comprises IL-10. In some embodiments, the therapeutic molecule comprises an endothelin-converting enzyme (ECE). In some embodiments, the therapeutic molecule comprises a protease enzyme, e.g., cathepsin B or cathepsin D. In some embodiments, the therapeutic molecule comprises cathepsin B. In some embodiments, the therapeutic molecule comprises cathepsin D. In some embodiments, the therapeutic molecule comprises matrix metalloproteinase (MMP) enzyme, e.g., MMP2 or MMP9. In some embodiments, the therapeutic molecule comprises matrix metalloproteinase 2 (MMP2). In some embodiments, the therapeutic molecule comprises matrix metalloproteinase 9 (MMP9). Particular therapeutic molecules are described in the following disclosures, which are incorporated herein in their entirety by reference: Leissring et al., Neuron 2003; 40:1087-1093; Eckman et al., J Biol Chem 2001; 276:24540-24548; Hernandez-Guillamon et al., J Biol Chem 2015; 290(24):15078-15091; Suire and Leissing, J Exp Neurol. 2021; 2(1):10-15; Wang et al., J Biol Chem 2012; 287(47):39843-41; Zhao et al., J Exp Med. 2022; 219(12):e20212479; Frenkel et al., Nat Commun. 2013; 4:2030; N'Songo et al., Mol Neurodegener. 2013; 8(Suppl 1):P33; Ulrich et al., J Exp Med. 2018; 215(4):1047-1058; Yi et al., Cytotechnology 2020; 72(4):589-602. In some embodiments, the therapeutic molecule comprises a metalloprotease, which may comprise neprilysin activity. In some embodiments, the therapeutic molecule comprises neprilysin. In some embodiments, the therapeutic protein comprises neprilysin or a derivative thereof. For example, the therapeutic protein may comprise an amino acid sequence at least 70%, at least 75%, at least 80%, at least 85%, at least 90%, at least 95%, at least 96%, at least 97% at least 98%, or at least 99% identical to SEQ ID NO: 1, SEQ ID NO: 2, or SEQ ID NO: 3. In some embodiments, the therapeutic protein comprises an amino acid sequence selected from the group consisting of SEQ ID NO: 1, SEQ ID NO: 2, and SEQ ID NO: 3. For example, in some embodiments, the therapeutic protein comprises a membrane-bound neprilysin according to SEQ ID NO: 1. In some embodiments, the therapeutic protein comprises a membrane-bound neprilysin according to SEQ ID NO: 2. In some embodiments, the therapeutic protein comprises a secreted neprilysin according to SEQ ID NO: 3.

The pathology-responsive promoter may, but need not be, an endogenous promoter. In some embodiments, the promoter is an endogenous promoter. In some embodiments, the promoter is not an endogenous promoter. In some embodiments, the promoter is an endogenous promoter but is also incorporated into the cell exogenously (separately), e.g., as a proximal promoter, wherein the therapeutic molecule is operatively linked to the proximal promoter. In some embodiments, the promoter is the wild type (non-modified) form of the promoter. In some embodiments, the promoter is modified (as compared to its wild type form). As a non-limiting example, a CD9 proximal promoter may be produced and integrated within the AAV safe harbor locus.

The pathology-responsive promoter may comprise a promoter from a gene selected from the group consisting of a DCSTAMP gene, a CD9 gene, a CD44 gene, an LGALS3 gene, an SPP1 gene, a GPNMB gene, an HLA-DRB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, an ITGAX gene, and a MITF gene. For example, in some embodiments, the promoter comprises the promoter of a CD9 gene. In some embodiments, the promoter comprises the promoter of an LGALS3 gene. In some embodiments, the promoter comprises the promoter of a HLA-DRB gene. In some embodiments, the promoter comprises the promoter of the CD11c (ITGAX) gene. In some embodiments, the promoter comprises the promoter of a gene selected from: CD9, LGALS3, HLA-DRB, and CD11c. In some embodiments, the promoter comprises the promoter of a gene selected from: CD9, LGALS3, HLA-DRB, TREM2, and CD11c. In some embodiments, the promoter comprises the promoter of a DCSTAMP gene. In some embodiments, the promoter comprises the promoter of a CD44 gene. In some embodiments, the promoter comprises the promoter of an SPP1 gene. In some embodiments, the promoter comprises the promoter of a GPNMB gene. In some embodiments, the promoter comprises the promoter of an LPL gene. In some embodiments, the promoter comprises the promoter of a LIPA gene. In some embodiments, the promoter comprises the promoter of a FABP3 gene. In some embodiments, the promoter comprises the promoter of an MS4A6A gene. In some embodiments, the promoter comprises the promoter of a CXCR4 gene. In some embodiments, the promoter comprises the promoter of a CH13L1 gene. In some embodiments, the promoter comprises the promoter of an OLR1 gene. In some embodiments, the promoter comprises the promoter of a CD36 gene. In some embodiments, the promoter comprises the promoter of a SLAMF8 gene. In some embodiments, the promoter comprises the promoter of a TREM2 gene. In some embodiments, the promoter comprises the promoter of an MSR1 gene. In some embodiments, the promoter comprises the promoter of a B2M gene. In some embodiments, the promoter comprises the promoter of a MITF gene. The present invention is not limited to the aforementioned promoters. While the data is not shown for the promoters such those from a DCSTAMP gene, a CD44 gene, an SPP1 gene, a GPNMB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, and a MITF gene, one of ordinary skill in the art understands that these promoters may be used in lieu of those from CD9, LGALS3, HLA-DRB, and CD11c with a reasonable expectation of success.

Other non-limiting examples of pathology-responsive promoters useful for producing a modified cell of the disclosure include, but are not limited to, HLA-DRB3, HLA-DRB5, HLA-DRA, CHIT1, DKK2, HLA-DQB1, PLXNA1, CCR7, HLA-DPA1, RGCC, COX6A2, HLA-DRB1, HLA-DPB1, TAFA3, FAIM2, ZNF804A, LGALS1, DOK2, HLA-DOA, GPR153, SLPI, ADGRF5, MYOZ1, HBEGF, BHLHE40, TMEM37, SLC16A8, GRM5, S100A4, GABRB1, LONRF3, PIGR, RAB7B, BIRC7, FABP5, CA2, FCMR, GABRB3, ATF7-NPFF, ANXA2, EGR2, OR9G1, IFNLR1, APOC1, CD200R1, PKD2L1, TDRD6, HLA-DMA, KCNJ5, GLDN, PREX2, OLFM2, PADI2, COL1A2, RRAGD, ROR2, PTCRA, EDN1, RAMP1, BCL2A1, CD83, CHST2, DAGLA, MAFB, DUSP2, FPR3, DPYD, PHLDA1, PTPRG, IER5L, CPNE8, MPDZ, RNF152, PTGER4, MCF2, SDS, CIITA, RGS16, ANGPTL6, PLA2G7, CCL3, ATF3, RAB42, TBX18, STARDI3, NR4A3, S100A13, TRDN, GADD45G, ADRA1B, LYPD1, FXYD6, EPHB6, FERMT2, ART4, TNFSF18, TNFSF15, SLC38A4, CDH6, SCIN, FGR, ZNF385B, FUT9, SORCS1, KLF10, CFD, TRPM1, FLVCR2, NFIL3, MYO1E, ITPRID1, SLC47A1, LITAF, RASGEF1B, DENND2D, ADCY3, SYTL3, ARL4C, IL4I1, CDCP1, TRERF1, VAT1, INPP1, ARRDC4, ENPP1, GSDME, APOC2, COL4A1, CDKN1A, KCNC4, KCNJ2, USP2, UNC13B, VEGFB, TNFAIP2, SLC35F2, HLA-DMB, NAB2, ACP5, CYTL1, FXYD5, ALDH1A2, TMEM176B, TAGLN2, TIMD4, MEP1A, ADGRL2, FAM20C, LRRC39, CD74, RASGRP3, HEG1, TNFRSF14, ADGRE2, FAM110B, GADD45B, TSKU, ID2, ADGRD1, DUSP10, TNFSF13B, FKBP5, MYC, BCL6, TNFAIP8L3, ARID5B, APOC4-APOC2, PMAIP1, P2RY8, CCRL2, RNASE1, LSP1, ARHGEF3, CLEC19A, KCNQ5, DTNA, PILRA, WIPF3, CSTB, XYLT1, DBI, SDC2, KCNMA1, ALCAM, TSPAN4, RPS28, KLHDC8B, MBOAT1, FAM20A, CDH8, NAP1L1, SMIM4, ALAS1, PDLIM7, IQGAP2, INPP4B, TRIM58, SLC36A1, TXN, HLA-B, HSPB1, AP1S2, PLAU, HLA-DQA1, CYSTM1, IER5, MS4A7, SPN, CTSD, SELENOP, SDSL, CD14, HLA-F, B3GALNT1, GAPT, RASSF3, GAS7, GAS2L3, GPD2, CPM, SGPP1, LILRA4, NEB, PRXL2C, SNX24, PPARG, CAPG, CD6, INHBA, PTPN7, LYRM9, ATOX1, UGCG, TMSB10, IFI30, M1AP, SH3BGRL3, CMYA5, PRKCH, NIBAN1, GK, HSD3B7, GPR65, LGALS9, RPL36, SH3RF1, WWC3, ALDH5A1, SNTB2, HACD4, LTA4H, SCIMP, SLC11A1, PTPDC1, CPNE2, CALHM2, PRDX1, APOE, IQGAP1, OSBPL3, GRK3, DPEP2, TMEM154, FOXP1, GYPC, SLC15A3, NENF, HPSE, TNFRSF11A, PPM1M, HPCAL1, SLC46A1, SLC1A3, MORC1, GABARAPL1, MGLL, TGIF1, ZYX, MAP4, or MFSD2A.

The nucleic acid sequence encoding the therapeutic molecule may be inserted into the genome of the modified cell (see Example 3). The nucleic acid sequence may be inserted into the genome of the modified cell so that transcription of the nucleic acid sequence is under the control of an endogenous pathology-responsive promoter in the genome of the modified cell. The site of insertion of the nucleic acid sequence may be within, upstream, or downstream of a gene controlled by a pathology-responsive promoter, such that the nucleic acid sequence encoding the therapeutic molecule is in-frame with a coding sequence in an exon of the gene. The nucleic acid sequence may be inserted within a gene controlled by a pathology-responsive promoter, such that the nucleic acid sequence encoding the therapeutic molecule is joined with at least part of the coding sequence of an exon of the gene. The exon may be any exon within the gene, including the first or last exon. A first polynucleotide encoding a protease cleavage site, a ribosomal skipping sequence, or a self-cleaving peptide may be inserted between the coding sequence of the exon and the therapeutic molecule encoding the nucleic acid sequence. As an example, in some embodiments, the self-cleaving peptide comprises a P2A sequence. To produce a secreted therapeutic molecule, a second polynucleotide encoding a secreted peptide signal sequence may be joined with the nucleic acid sequence encoding the therapeutic molecule. In aspects of the disclosure in which the therapeutic molecule is a therapeutic protein, the coding sequence of the exon and the therapeutic protein-encoding nucleic acid sequence may be joined in-frame. Likewise, the first polynucleotide encoding the protease cleavage site, a ribosomal skipping sequence, or self-cleaving peptide, and the second polynucleotide encoding the secreted peptide signal sequence may be joined in-frame with one another and with the coding sequence of the exon and the therapeutic protein-encoding nucleic acid sequence.

Modified cells of the disclosure may be produced using a cell from a lineage of cells that can differentiate into migratory cells. For example, in some embodiments, the modified cell is produced using a pluripotent stem cell (PSC). In some embodiments, the modified cell is produced using an induced pluripotent stem cell (iPSC). In some embodiments, the modified cell is produced using a myeloid progenitor cell. In some embodiments, the modified cell is produced using an erythromyeloid progenitor. In some embodiments, the modified cell is produced using a hematopoietic stem cell. In some embodiments, the modified cell is produced using a hematopoietic progenitor or precursor cell (HSPC). In some embodiments, the modified cell is produced using a lymphoid progenitor cell. In some embodiments, the modified cell is produced using a megakaryocyte-erythroid (mk-ery) cell. In some embodiments, the modified cell is produced using a cord-blood stem cell. In some embodiments, the modified cell is produced using an embryonic stem cell. In some embodiments, the modified cell is produced using a myeloid progenitor cell, a hematopoietic stem cell, or a hematopoietic progenitor cell. In some embodiments, the modified cell is produced using a pluripotent stem cell (PSC), an induced pluripotent stem cell (iPSC), a myeloid progenitor cell, an erythromyeloid progenitor, a hematopoietic stem cell, a hematopoietic progenitor or precursor cell (HSPC), a lymphoid progenitor cell, a megakaryocyte-erythroid (mk-ery) cell, a cord-blood stem cell, or an embryonic stem cell.

The modified cell may be a microglia-like (MGL) cell, which may be an induced MGL (iMGL), which may be a human induced pluripotent stem-cell-derived MGL cell (hiMGL). The hiMGL may express P2RY12 and TREM2. The hiMGL may express TMEM119 or Iba1. The hiMGL may express higher levels of AXL, STAB1, P2RY6, CCR6, or GPR84 than mature microglia endogenous to the individual. The hiMGL may express lower levels of CTSL, CTSD, or NPL than mature microglia endogenous to the individual. The hiMGL may express reduced levels of FFAR2 and COL26A1 than mature microglia endogenous to the individual. The hiMGL may express reduced levels of Siglec11 and Siglec12 than mature microglia endogenous to the individual. The hiMGL may express P2RY13 or OLFML3. The hiMGL may be capable of phagocytosing β-amyloid (Aβ) peptide plaques, soluble Aβ monomers, insoluble Aβ monomers, Aβ oligomers, pyroglutamate Aβ, protofibrils, and/or fibrils comprising Aβ of varying lengths. The hiMGL may be capable of phagocytosing Aβ-40 or Aβ-42 of either soluble or insoluble form.

One aspect of the disclosure provides a composition comprising a modified cell of the disclosure.

One aspect of the disclosure provides a method of treating Alzheimer's Disease in an individual in need thereof, or ameliorating symptoms or pathological processes associated with Alzheimer's disease in said individual. The method comprises administering to the individual a modified cell of the disclosure or composition of the disclosure. The method comprises administering to (e.g., transplanting into) a brain tissue of the individual a modified cell of the disclosure or composition of the disclosure (e.g., hiMGLs).

One aspect of the disclosure provides a method of treating Parkinson's disease in an individual in need thereof, or ameliorating symptoms or pathological processes associated with Parkinson's disease in said individual. The method comprises administering to the individual a modified cell of the disclosure or composition of the disclosure. The method comprises administering to (e.g., transplanting into) a brain tissue of the individual a modified cell of the disclosure or composition of the disclosure (e.g., hiMGLs).

One aspect of the disclosure provides a method of treating Huntington's disease in an individual in need thereof, or ameliorating symptoms or pathological processes associated with Huntington's disease in said individual. The method comprises administering to the individual a modified cell of the disclosure or composition of the disclosure. The method comprises administering to (e.g., transplanting into) a brain tissue of the individual a modified cell of the disclosure or composition of the disclosure (e.g., hiMGLs).

One aspect of the disclosure provides a method of treating amyotrophic lateral sclerosis (ALS) in an individual in need thereof, or ameliorating symptoms or pathological processes associated with ALS in said individual. The method comprises administering to the individual a modified cell of the disclosure or composition of the disclosure. The method comprises administering to (e.g., transplanting into) a brain tissue of the individual a modified cell of the disclosure or composition of the disclosure (e.g., hiMGLs).

One aspect of the disclosure provides a method of treating a neurodegenerative disorder, e.g., an amyloid beta-related neurodegenerative disorder, in an individual in need thereof, or ameliorating symptoms or pathological processes associated with ALS in said individual. The method comprises administering to the individual a modified cell of the disclosure or composition of the disclosure. The method comprises administering to (e.g., transplanting into) a brain tissue of the individual a modified cell of the disclosure or composition of the disclosure (e.g., hiMGLs).

One aspect of the disclosure provides a kit comprising the modified cell or composition of the disclosure. For example, the kit may comprise a modified cell described herein, wherein the modified cell comprises a nucleic acid sequence encoding the therapeutic molecule as described herein.

One aspect of the disclosure provides a method of reducing the amount of Aβ peptide in Aβ peptide plaques; and/or, the size or number of soluble Aβ monomers, insoluble Aβ monomers, Aβ oligomers, pyroglutamate Aβ, protofibrils, or fibrils comprising Aβ of varying lengths, in the brain of the individual; the method comprising administering a modified cell or a composition of the disclosure to the individual.

One aspect of the disclosure is a method of producing a modified cell of the disclosure, the method comprising introducing into a cell a nucleic acid sequence encoding a therapeutic molecule, wherein the nucleic acid sequence is operatively linked to a promoter that is responsive to amyloid beta-related pathology, and wherein the therapeutic protein alters an amyloid beta-related pathology phenotype or at least one aspect of the amyloid beta-related pathology.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A-1P illustrates the relative locations of HLA-DRB, CD9 (tetraspanin), CD11c (ITGAX), and LGALS3 expression, and amyloid plaques in xMGs that are proximal vs. distal to fibrillar amyloid plaques.

FIGS. 1A, 1E, 1I, and 1M show beta-amyloid (Aβ) expression. FIGS. 1B, 1F, 1J, & 1N show xMGs expressing cytosolic green fluorescent protein (cytoGFp) that was used as a genetic label of the human cells. FIGS. 1C, 1G, 1K, & 1O show expression of HLA-DRB, CD9, CD11c, and LGALS3 proteins, respectively. FIGS. 1A, 1E, 1I & 1M show the location of fibrillar amyloid plaques. FIG. 1D shows an overlay of FIGS. 1A, 1B, & 1C; FIG. 1H shows an overlay of FIGS. 1E, 1F & 1G; FIG. 1L shows an overlay of FIGS. 1I, 1J & 1K; FIG. 1P shows an overlay of FIGS. 1M, 1N & 1O.

Brains were fixed with 4% paraformaldehyde and then cut into 40 micron coronal sections using a freezing microtome. We then utilized immunofluorescence labeling with antibodies against the designated proteins and Amylo-Glo which recognized fibrillar beta-amyloid plaques. Confocal microscopy was then used to visualize human microglial proteins and amyloid pathology.

FIG. 2A-2H are similar to FIGS. 1E-1H but show a closer view of the relative locations of CD9 (tetraspanin) expression, and amyloid plaques in xMGs that are proximal or distal to fibrillar amyloid plaques. FIGS. 1A & 1E show xMGs expressing cytosolic green fluorescent protein (cytoGFp) that was used as a genetic label of the human cells. FIGS. 1B & 1F show expression of CD9 protein. FIGS. 1C & 1G show the location of fibrillar amyloid plaques. FIGS. 1E, 1F, & 1G show a higher power view of FIGS. 1A, 1B, & 1C, respectively. FIG. 1D shows an overlay of FIGS. 1A, 1B, & 1C, while; FIG. 1H shows an overlay of FIGS. 1E, 1F & 1G.

FIGS. 3A & 3B show the design used to modify the genome of human iPSCs in which neprilysin (NEP) is inserted downstream of the endogenous CD9 locus using a P2A element, such that CD9 expression remains under the control of the endogenous CD9 promoter and NEP expression is also co-regulated and co-expressed under control of the CD9 promoter. FIG. 3A shows a construct in which the NEP is membrane-anchored. FIG. 3B shows a construct in which NEP is secreted (sNEP). SP=secreted peptide signal sequence. P2A=self-cleaving peptide used to enable polycistronic expression.

FIG. 4A-4C show microglial cells according to the present disclosure digesting and phagocytosing fluorescently labeled fibrillarized human Aβ42 amyloid in vitro. FIG. 4A illustrates membrane-anchored neprilysin expressing microglial cells digesting and phagocytosing digested Aβ. FIG. 4B illustrates secreted neprilysin expressing microglial cells digesting and phagocytosing digested Aβ. FIG. 4C shows Aβ phagocytosis by wild type (WT) microglial cells (wild type cells differentiated from human parental iPSC line and not CRISPR-edited to include neprilysin construct), membrane-anchored neprilysin expressing microglial cells (NEP), and secreted neprilysin expressing microglial cells (sNEP). Increases on the Y-axis over time indicate increasing internalization and phagocytosis of Aβ. Decreasing slope over time, as observed in sNEP and NEP data, indicates increasing degradation of Aβ following internalization.

FIG. 5A-5C show expression of Neprilysin within the cortex and hippocampus of wildtype (WT) or amyloid-plaque developing AD mice (5×-MITRG). FIGS. 5A and 5B depict levels of human Neprilysin in soluble cortex and hippocampus brain extracts, respectively, from mice transplanted with PBS (n=13), wild-type microglia (WT; n=9), membrane-bound Neprilysin-expressing microglia (NEP; n=11), and secreted Neprilysin-expressing microglia (sNEP; n=9) analyzed by Human Neprilysin DuoSet ELISA. FIG. 5C depicts levels of Neprilysin (monoclonal antibody, SN5C) normalized to GAPDH levels per lane (n=3) from soluble hippocampus samples of 5×-MITRG (5×) and MITRG-WT (WT) transplanted mice. This antibody preferentially detects membrane-bound NEP, but not sNEP. This Western blot confirms a significant upregulation of NEP levels only in 5×-MITRG mice transplanted with CD9-NEP microglial progenitors. Data are represented as mean value±SEM. Statistical significance is calculated using a two-way ANOVA test. ns=not significant or p≥0.05; * p<0.05; ** p<0.01; *** p<0.001; **** p<0.0001.

FIG. 6A-6D show reductions in soluble amyloid species in brains treated with microglial cells of the present disclosure. Levels of Human Aβ-42 and Aβ-40 peptide were measured in soluble extracts of cortex (FIGS. 6A, 6C) and hippocampus (FIGS. 6B and 6D) samples taken from mice transplanted with PBS (n=13), wild-type microglia (WT; n=9), membrane-bound Neprilysin-expressing microglia (NEP; n=11), and secreted Neprilysin-expressing microglia (sNEP; n=9) analyzed by MSD Assay. Data are represented as mean value±SEM. Statistical significance is calculated using ANOVA with Tukey's post hoc test. ns=not significant or p≥0.05; * p<0.05; ** p<0.01; *** p<0.001; **** p<0.0001.

FIG. 7A-7D show reductions in insoluble amyloid species in brains treated with microglial cells of the present disclosure. Insoluble amyloid species provide a biochemical measure of amyloid plaques, and Aβ-42 is considered the more neurotoxic and aggregation-prone species of Aβ. Human Aβ-42 and Aβ-40 peptides were measured in insoluble extracts of the cortex (FIGS. 7A, 7C) and hippocampus (FIGS. 7B and 7D) samples taken from mice transplanted with PBS (n=13), wild-type microglia (WT; n=9), membrane-bound Neprilysin-expressing microglia (NEP; n=11), and secreted Neprilysin-expressing microglia (sNEP; n=9) analyzed by Mesoscale Devices (MSD) Aβ multiplex Assay. Data are represented as mean value±SEM. Statistical significance is calculated using ANOVA with Tukey's post hoc test. ns=not significant or p<0.05; * p≥0.05; ** p<0.01; *** p<0.001; **** p<0.0001.

FIG. 8A-8B show reductions of soluble Aβ oligomers in brains treated with microglial cells of the present disclosure. Human Aβ oligomers collected from soluble extracts of cortex (FIG. 8A) and hippocampus (FIG. 8B) samples from mice transplanted with PBS (n=13), wild-type microglia (WT; n=9), membrane-bound Neprilysin-expressing microglia (NEP; n=11), and secreted Neprilysin-expressing microglia (sNEP; n=9) were analyzed by MSD Assay. Data are represented as mean value±SEM. Statistical significance is calculated using ANOVA with Tukey's post hoc test. ns=not significant or p z 0.05; * p<0.05; ** p<0.01; *** p<0.001; **** p<0.0001.

FIG. 9A-9H show that delivery of Neprilysin according to the methods of this disclosure has no effect on synaptic density in wildtype MITRG mice (FIGS. 9A-9D) but reduces the synaptic loss that occurs in 5×-MITRG (9E-9H) mice. Levels of presynaptic marker synaptophysin (SYP1) and postsynaptic marker PSD-95 in soluble cortex (9A, 9C, 9E, 9G) and hippocampus (9B, 9D, 9F, 9H) samples from mice transplanted with PBS (n=13), wild-type microglia (WT; n=9), membrane-bound Neprilysin-expressing microglia (NEP; n=11), and secreted Neprilysin-expressing microglia (sNEP; n=9) were analyzed by ELISA. Data are represented as mean value±SEM. Statistical significance is calculated using ANOVA with Tukey's post hoc test. ns=not significant or p≥0.05; * p<0.05; ** p<0.01; *** p<0.001; **** p<0.0001.

FIG. 10A-10B show delivery of Neprilysin according to the methods of this disclosure reduces astrogliosis in 5×-MITRG mice. Levels of glial fibrillary acidic protein (GFAP) in soluble cortex (FIG. 10A) and hippocampus (FIG. 10B) samples from 5×-MITRG mice transplanted with PBS (n=13), wild-type microglia (WT; n=9), membrane-bound Neprilysin-expressing microglia (NEP; n=11), and secreted Neprilysin-expressing microglia (sNEP; n=9) analyzed by ELISA are shown. Data are represented as mean value±SEM. Statistical significance is calculated using ANOVA with Tukey's post hoc test. ns=not significant or p≥0.05; * p<0.05; ** p<0.01; *** p<0.001; **** p<0.0001.

FIG. 11A-11D show targeted induction of Neprilysin in response to Aβ Pathology in microglial cells of this disclosure limits off-target degradation of additional neuropeptide substrates of Neprilysin in vivo. Levels of non-targeted Neprilysin substrates Bradykinin (FIGS. 11A-11B) and Somatostatin (FIGS. 11C-11D) in soluble cortex (FIGS. 11A, 11C) and hippocampus (FIGS. 11B, 11D) samples from mice transplanted with PBS (n=13), wild-type microglia (WT; n=9), membrane-bound Neprilysin-expressing microglia (NEP; n=11), and secreted Neprilysin-expressing microglia (sNEP; n=9) were analyzed by ELISA. Data are represented as mean value±SEM. Statistical significance is calculated using ANOVA with Tukey's post hoc test. ns=not significant or p≥0.05; * p<0.05; ** p<0.01; *** p<0.001; **** p<0.0001.

FIGS. 12A and 12B shows, in schematic form, the design of in vivo experiments described in this disclosure.

FIG. 13 shows amyloid pathology induces CD9 expression within human microglia which leads to highly localized induction of the Neprilysin payload. The nuclei of many transplanted human microglia are shown (Ku80). However, CD9 expression and thus Neprilysin induction is restricted to those human microglia that are adjacent to Amylo-Glo positive beta-amyloid plaques.

FIG. 14 shows Hippocampal Aβ Pathology is reduced by sNEP-expressing human microglia in vivo.

FIG. 15 shows the numbers of medium and large AmyloGlo positive plaques are significantly reduced in vivo. Statistical significance is calculated using ANOVA with Tukey's post hoc test. ns=not significant or p≥0.05; * p<0.05; ** p<0.01; *** p<0.001; **** p<0.0001.

FIG. 16A-16D show the effect of human iPSC-microglia transplantation on Aβ proteins in 5×-MITRG mice. Levels of Human Aβ-42 (FIGS. 16A-16B) and Aβ-40 (FIG. 16C-16D) peptides collected from soluble extractions of cortex (FIG. 16A, 16C) and hippocampus (FIG. 16B, 16D) samples from mice transplanted with PBS (n=13) and wild-type microglia (WT; n=9) analyzed by MSD Assay. Data are represented as mean value±SEM. Statistical significance calculated using unpaired T-test. ns=not significant or p≥0.05; * p<0.05; ** p<0.01; *** p<0.001; **** p<0.0001.

FIG. 17A-17D show the effect of human iPSC-microglia transplantation on Aβ proteins in 5×-MITRG mice. Levels of Human Aβ-42 (FIGS. 17A-17B) and Aβ-40 (FIG. 17C-17D) peptides collected from insoluble extractions of cortex (FIG. 17A, 17C) and hippocampus (FIG. 17B, 17D) samples from mice transplanted with PBS (n=13) and wild-type microglia (WT; n=9) analyzed by MSD Assay. Data are represented as mean value±SEM. Statistical significance calculated using unpaired T-test. ns=not significant or p≥0.05; * p<0.05; ** p<0.01; *** p<0.001; **** p<0.0001.

FIG. 18A-18D show the effect of targeted delivery of Neprilysin on Aβ proteins in 5×-MITRG mice. Levels of Human Aβ-42 (FIGS. 18A-18B) and Aβ-40 (FIG. 18C-18D) peptide collected from soluble extractions of cortex (FIG. 18A, 18C) and hippocampus (FIG. 18B, 18D) samples from mice transplanted wild-type microglia (WT; n=9), membrane-bound Neprilysin-expressing microglia (NEP; n=11), and secreted Neprilysin-expressing microglia (sNEP; n=9) analyzed by MSD Assay. Data are represented as mean value±SEM. Statistical significance is calculated using ANOVA with Tukey's post hoc test. ns=not significant or p≥0.05; * p<0.05; ** p<0.01; *** p<0.001; **** p<0.0001.

FIG. 19A-19D show the effect of targeted delivery of Neprilysin on Aβ proteins in 5×-MITRG mice. Levels of Human Aβ-42 (FIGS. 19A-19B) and Aβ-40 (FIG. 19C-19D) peptides collected from soluble extractions of cortex (FIG. 19A, 19C) and hippocampus (FIG. 19B, 19D) samples from mice transplanted wild-type microglia (WT; n=9), membrane-bound Neprilysin-expressing microglia (NEP; n=11), and secreted Neprilysin-expressing microglia (sNEP; n=9) analyzed by MSD Assay. Data are represented as mean value±SEM. Statistical significance is calculated using ANOVA with Tukey's post hoc test. ns=not significant or p≥0.05; * p<0.05; ** p<0.01; *** p<0.001; **** p<0.0001.

FIG. 20A-20D shows volcano plots from bulk RNA sequencing of four independent xMG lines. 5×-MITRG mouse pups were transplanted with human microglial progenitors generated from four independent induced pluripotent stem cell lines. At 6-months of age, xMGs were isolated from the brains of 5×-MITRG mice and subpopulations of xMGs separated via fluorescent-activated cell sorting (FACS). To enrich plaque-associated disease-associated microglia (DAMs), xMGs were FACS sorted to isolate CD9/HLA-DRB double positive microglia. In contrast homeostatic microglia were isolated as being negative for both CD9 and HLA-DRB expression. RNA was isolated from each of these samples and then analyzed via bulk RNA sequencing. The resulting bulk sequencing comparison between homeostatic and DAM xMG subpopulations revealed numerous genes that were significantly upregulated within DAM microglia. Nearly all DAM genes previously identified via single cell sequencing were again identified as being enriched within the bulk sequenced DAM subpopulation. However, because bulk sequencing provided far greater sequencing depth, many other significantly enriched DAM genes were identified including those provided in [0020]. FIG. 20A-20D provide volcano plots from these bulk sequencing comparisons from xMGs derived from each of the four independent induced pluripotent stem cell lines and a subset of significantly enriched genes are labeled. This analysis provided the more complete dataset of plaque-induced human microglia genes as described herein.

These plots compare flow cytometry (FACs) sorted CD9+/HLA-DRB+ double positive plaque-associated microglia to double negative homeostatic microglia. A subset of significant genes (FDR≤0.05) with a log₂(fold-change) ≥1 (at least 2-fold increase) when comparing DAM vs Homeostatic subpopulations across the patient lines are shown in the plots, and the rest are described below.

### DETAILED DESCRIPTION OF THE INVENTION

The present disclosure relates to compositions for, and methods of, treating diseases or conditions associated with amyloid beta-related pathology, such as but not limited to Alzheimer's disease, Parkinson's disease, Huntington's disease, amyotrophic lateral sclerosis (ALS), etc. More specifically, the present disclosure describes cells that have been modified to express (and present or secrete) at least one therapeutic molecule that can alter at least one amyloid beta-related pathology phenotype. Such cells have been modified so that the therapeutic molecule is expressed when the modified cell contacts or is proximate to amyloid beta-related pathology. Thus, an embodiment of the disclosure can generally be practiced by producing cells that express a therapeutic molecule when the cells are in contact with or proximate to amyloid beta-related pathology. Accordingly, the present disclosure also describes methods of making such cells, and of using such cells to treat disease.

Before the present disclosure is further described, it is to be understood that the invention is not limited to particular embodiments described, as such may, of course, vary. For example, the present invention is not limited to embodiments associated with Alzheimer's Disease. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. For example, a compound refers to one or more compound molecules. As such, the terms “a,” “an,” “one or more,” and “at least one” can be used interchangeably. Similarly, the terms “comprising,” “including,” and “having” can be used interchangeably. It is further noted that the claims may be drafted to exclude any optional element. As such, this statement is intended to serve as an antecedent basis for use of such exclusive terminology as “solely,” “only” and the like in connection with the recitation of claim elements or use of a “negative” limitation.

Publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed. All publications mentioned herein are incorporated herein by reference to disclose and describe the methods and/or materials in connection with which the publications are cited.

The present disclosure relates to modified cells that express a therapeutic molecule for treating diseases associated with amyloid beta-related pathology when the cells are in contact with, or proximate to amyloid beta-related pathology. As used herein, a “modified cell” is a cell that has been intentionally altered using, for example, recombinant DNA technology, CRISPR technology, and the like. Modified cells of the disclosure may be produced, for example, by introducing into the cell at least one nucleotide sequence encoding a therapeutic molecule that can alter at least one amyloid beta-related pathology phenotype, wherein the nucleic acid sequence is inserted into the cell in such a manner that transcription of the nucleotide sequence occurs when the cell is proximal to or in contact with amyloid beta-related pathology.

Cells used to produce modified cells of the disclosure may be obtained commercially (i.e., purchased), obtained from cell culture (e.g., of previously banked cells), or they may be obtained from an individual. As used herein, the terms “individual,” “subject,” and “patient” are well-recognized in the art and are herein used interchangeably to refer to any human or other animal that may be treated using cells of the disclosure. Examples include, but are not limited to, humans and other primates, including non-human primates such as chimpanzees and other apes and monkey species; farm animals such as cattle, sheep, pigs, seals, goats, and horses; domestic mammals such as dogs and cats; and laboratory animals including rodents such as mice, rats, and guinea pigs. The terms “individual,” “subject,” and “patient” by themselves do not denote a particular age, sex, race, and the like. Thus, individuals of any age, whether male or female, are intended to be covered by the present disclosure and include, but are not limited to the elderly, adults, children, babies, infants, and toddlers. Likewise, unless specified otherwise, cells and methods of the present disclosure can be from and/or applied to any race, including, for example, Caucasian (white), African-American (black), Native American, Native Hawaiian, Hispanic, Latino, Asian, African (e.g., of African descent) and European.

Any type of cell that can be modified to express a therapeutic molecule when the cell is proximate to, or in contact with, Alzheimer's disease-related pathology may be used to produce modified cells of the disclosure. In some aspects, modified cells are produced using a cell from a lineage of cells that can differentiate into migratory cells. Examples of cells useful for producing modified cells of the disclosure include, but are not limited to, pluripotent stem cells (PSCs), induced pluripotent stem cells (iPSCS), myeloid progenitor or precursor cells, erythromyeloid progenitors, hematopoietic stem and progenitor cells (HSPCs), a cord-blood stem cell, lymphoid progenitor cells, and megakaryocyte-erythroid (mk-ery) progenitor cells. In some aspects, a cell used to produce modified cells of the disclosure may be a pluripotent stem cell (PSC), including induced PSCs (iPSCS), a progenitor cell, an embryonic stem cell, or cells derived therefrom. As used herein, “pluripotent stem cell” (PSC) refers to a cell that has the capacity to self-renew by division, and to develop into the three primary germ cell layers of an early embryo, and therefore into all cells of an adult body. As used herein, “induced pluripotent stem cell” or “iPSC” refers to a type of pluripotent stem cell that can be generated directly from a somatic cell. In some aspects, a cell used to produce modified cells of the disclosure may be a circulating blood progenitor cell (e.g., a myeloid progenitor cell, CD34+ hematopoietic progenitor cell, or a monocyte).

In some aspects, a cell used to produce a modified cell of the disclosure is a microglial-like cell (MGL), including an induced MGL (iMGL) cell such as a human iMGL (hiMGL) or a microglial progenitor cell. As used herein, “microglial progenitor cell” refers to a biological cell that can differentiate into microglia or microglial-like cells. Microglial progenitor cells may include hematopoietic progenitor cells, erythromyeloid progenitor cells, primitive macrophages, and the like. Microglial progenitor cells may also be derived from pluripotent stem cells (PSCs), including induced pluripotent stem cells (iPSCs) and embryonic stem cells (ESCs). iMGLs are further described in US20200239844 and Abud et. al., Neuron, 2017: PMID: 28426964, the disclosures of which are incorporated herein by reference in their entirety. In some embodiments, iMGLs express microglial cell marker proteins such as P2RY12 and TREM2. In some embodiments, iMGLs have higher expression of AXL, P2RY6, CCR6, or GPR84 than adult microglia. In some embodiments, iMGLs have lower expression of CTSL, CTSD, or NPL than adult microglia. In some embodiments, iMGLs express mRNA sequences indicative of a mitotic cell cycle process gene ontology. In some embodiments, iMGLs reduce FFAR2 and COL26A1 expression when cultured with rat-hippocampal neurons. In some embodiments, iMGLs increase Siglec11 and Siglec12 expression when cultured with rat-hippocampal neurons. In some embodiments, iMGLs are differentiated in vitro. In some embodiments, iMGLs express P2RY13 and OLFML3. In some embodiments, iMGLs are capable of phagocytosing human synaptosomes. In some embodiments, iMGLs have less phagocytic activity on E. coli particles than macrophages. iMGLs are capable of phagocytosing amyloid Aβ fibers or tau oligomers. In some embodiments, IFNγ induces secretion of TNFα, IL-8, CCL2, and CCL4 from iMGLs. In some embodiments, IL-10 induces secretion of TNFα, IL-8, CCL3, CCL4, and CXCL10 from iMGLs. In some embodiments, ADP induces a transient calcium influx into the iMGLs. In some embodiments, iMGLs migrate in response to ADP. In some embodiments, iMGLs migrate into a human brain organoid (BORG). In some embodiments, iMGLs extend ramified processes with the BORG. In some embodiments, iMGLs cluster near an injury site in the BORG. In some embodiments, iMGLs are produced by contacting human induced hematopoietic progenitor cells (iHPCs) with a microglial differentiating medium comprising CSF-1, IL-34, and TGFβ1 or CSF-1, IL-34, and a TGFβ mimetic. In some embodiments, iMGLs are produced by plating human induced hematopoietic progenitor cells (iHPCs) on a basement membrane protein-coated culture dish; and contacting the human iHPCs with a microglial differentiating medium comprising CSF-1; IL-34; CSF-1, IL-34, and TGFβ1; or CSF-1, IL-34, and a TGFβ mimetic.

As used herein, “microglial-like cell” or “iMGL” refers to a microglial-like cell that resembles fetal and adult microglia that may be derived from pluripotent stem cells (PSCs), including iPSCs and embryonic stem cells (ESCs). As used herein, “microglia” refers to resident innate immune cells of the CNS that play a role in synaptic plasticity, neurogenesis, homeostatic functions, and immune activity.

Progenitor cells may be generated from PSCs, including iPSCs, using processes known in the art. Such progenitor cells may include but are not limited to, hematopoietic progenitor cells, erythromyeloid progenitor cells, or primitive macrophages.

As used herein, “iPSC-derived microglia-lineage cell” refers to human microglial-like cells (iMGLs) or microglial progenitor cells, that may be derived from iPSCs.

In various aspects, the iMGLs or microglial progenitor cells of the disclosure may be generated from PSCs using processes known in the art. In some aspects, the iMGLs or microglial progenitor cells of the disclosure may be generated from iPSCs or ESCs using processes known in the art. In some aspects, the microglial progenitor cells may include hematopoietic progenitor cells, erythromyeloid progenitor cells, or primitive macrophages. In some aspects, the iMGLs may be derived from microglial progenitor cells.

In some aspects, the iMGLs of the disclosure may be generated by the steps of: (i) differentiating PSCs using a media supplemented with hematopoietic differentiation factors to produce induced hematopoietic progenitor cells (iHPCs); (ii) isolating CD43+ iHPCs; (iii) differentiating the CD43+ iHPCs into human iMGLs using a microglial differentiating media; and (iv) maturing the iMGLs. In some aspects, HPC generation technology allows for collecting media enriched with precursors and carried to (iii) without isolating CD43+ iHPCs.

In some aspects, the human microglial-like cells (hiMGLs) of the disclosure may be generated by (i) differentiating PSCs using a media supplemented with hematopoietic differentiation factors; and (ii) differentiating the CD43+ iHPCs into iMGLs using a microglial differentiating media.

In some aspects, the human microglial-like cells (hiMGLs) of the disclosure may be produced from a cell of a first type comprising the steps of: (i) differentiating a cell of a first type into an iHPC; and (ii) differentiating the iHPC to produce an iMGL. In some aspects, the cell of a first type is not a PSC or an ESC.

In some aspects, the human microglial-like cells (hiMGLs) of the disclosure may be produced by a method comprising a step of differentiating an iHPC to produce an iMGL.

In some aspects, the human microglial-like cells (hiMGLs) of the disclosure may be produced by a method comprising a step of differentiating an engineered iHPC to produce an engineered hiMGL, wherein the engineered iHPC and engineered hiMGL express the therapeutic molecule (e.g., membrane-bound neprilysin, soluble neprilysin, etc.).

The iMGLs produced by any of the methods described herein may express any factor or any combination of factors that a typical canonical microglial cell expresses. In some aspects, the iMGLs produced are c-kit-/CD45+. In some aspects, the c-kit-/CD45+ iMGLs are detected using flow cytometry, immunofluorescence microscopy, qPCR, RNA-seq, or proteomics. In some aspects, other cell types are detected using flow cytometry, immunofluorescence microscopy, qPCR, RNA-seq, or proteomics. In some aspects, the iMGLs produced comprise two separate populations of iMGLs: (1) CD45+/CX3CR1− and (2) CD45+/CX3CR1+. In some aspects, the iMGLs produced are CD43+, CD235a+, or CD41+. In some aspects, the iMGLs produced are CD43+/CD235a+/CD41+.

In some aspects, TRIM14, CABLES1, MMP2, SIGLEC 11 and 12, MITF, and/or SLC2A5 mRNA and/or protein expression may be enriched in the produced iMGLs. In some aspects, COMT, EGR2, EGR3, and/or FFAR2 mRNA and/or protein expression is enriched in the produced iMGLs.

In some aspects, iMGLs may be provided that express a specific gene profile. Any of the iMGLs described herein may comprise a gene expression profile similar to canonical microglia cells. In some aspects, any of the compositions of iMGLs described herein comprise expression of any of the following genes: RUNX1, PU.1, CSF1R, CX3CR1, TGFBR1, RSG10, GAS6, PROS1, P2RY12, GPR34, C1Q, CR3, CABLES1, BHLHE41, TREM2, ITAM, APOE, SLCO2B1, SLC7A8, PPARD, C9orf72, GRN, LRRK2, TARDBP, and CRYBB1. Any of the iMGLs disclosed herein may comprise expression of any of these genes in any combination: RUNX1, SPI1, CSF1R, CX3CR1, TGFBR1, RSG10, GAS6, MERTK, PSEN2, PROS1, P2RY12, P2RY13, OLFML3, GPR34, C1Q, CR3, CABLES1, BHLHE41, TREM2, TYROBP, ITGAM, APOE, SLCO2B1, SLC7A8, PPARD, TMEM119, GPR56, C9orf72, GRN, LRRK2, TARDBP, and CRYBB1.

In some aspects, in any of the compositions of iMGLs described herein, TREM2 and P2RY12 may be co-expressed. In some aspects, any of the compositions of iMGLs described herein may not express any one or more of the genes KLF2, TREM1, MPT, ITGAL, and ADGRE5.

Cells used to produce modified cells of the disclosure may be autologous cells, allogeneic cells, or xenogeneic cells. The term “autologous” as used herein, refers to cells or tissues derived from one subject, wherein the one subject may be both a donor and a recipient. “Allogeneic,” as used herein, refers to cells of the same species that are genetically different from the cells being compared. The term “xenogeneic,” as used herein, refers to cells derived from a different species than the recipient. In certain embodiments, the modified cells may be autologous. In certain embodiments, the modified cells may be allogeneic. In certain embodiments, the modified cells may be xenogeneic.

In certain embodiments, the method comprises administering to (e.g., transplanting into) a brain tissue of the individual a modified cell of the disclosure or composition of the disclosure (e.g., hiMGLs). The methods disclosed herein may involve acquiring and modifying a cell from a subject and subsequently administering (e.g., transplanting) the modified cell, such as hiMGLs, back into the same subject. In other embodiments, the methods may involve obtaining and modifying a cell from a donor subject and administering (e.g., transplanting) the modified cell (e.g., hiMGLs) into a recipient subject. Additionally, in some embodiments, the methods may comprise acquiring and modifying a cell from a donor species and administering (e.g., transplanting) the modified cell (e.g., hiMGLs) into a recipient species.

As used herein, “nucleic acid molecule, “nucleic acid,” “nucleic acid sequence,” and the like, refer to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules or sequences may be composed of monomers that are naturally occurring nucleotides (such as DNA and RNA), or analogs of naturally occurring nucleotides (e.g., α-enantiomeric forms of naturally occurring nucleotides), or a combination of both. Nucleic acid molecules or sequences may be either single-stranded or double-stranded.

Introduction of a nucleic acid molecule or sequence into a cell to produce a modified cell may be achieved using nucleic acid transfer methods known in the art. In some aspects, the nucleic acid molecule or sequence may be introduced into cells using transfection, including virus vector-mediated transfection, conjugation, electroporation, liposome-mediated gene transfer, transduction, and direct transfer methods, such as microinjection or particle bombardment. The nucleic acid molecule or sequence may be a double or single-stranded linear nucleic acid molecule or sequence or a circular nucleic acid molecule or sequence. In some aspects, the nucleic acid molecule or sequence may be in a vector, such as a plasmid or a viral vector.

In some aspects, the nucleic acid or sequences comprises a nucleic acid vector. The terms “nucleic acid vector,” “vector,” and the like, are used herein to refer to a nucleic acid molecule or sequence capable of transferring or carrying another nucleic acid molecule or sequence. The nucleic acid to be transferred may be generally linked to, e.g., inserted into a vector nucleic acid molecule or sequence. A vector may include sequences that direct autonomous replication in the cell or may include sufficient sequences to allow integration into host cell DNA. Useful vectors may include, for example, plasmids (e.g., DNA or RNA plasmids), transposons, cosmids, bacterial artificial chromosomes, and viral vectors. Useful viral vectors may include, for example, replication-defective retroviruses and lentiviruses. In some aspects, the nucleic acid vector comprising the nucleic acid molecule or sequence may remain in the cytoplasm of the modified cell.

In some aspects, the nucleic acid sequence may be inserted into the genome of the modified cell. In some aspects, the nucleic acid sequence may be inserted into the genome of the cell to form a “synthetic allele” of a cellular gene. The term “synthetic allele” refers to an allele of a gene in the genetic material of a cell that is modified relative to one or both alleles of the same gene in the genetic material of a reference cell from the same subject. In one non-limiting example, the reference cell is a diploid germ-line cell from the same subject; in another non-limiting example, the reference cell is a cell taken from the subject prior to a therapeutic intervention, according to this disclosure. Synthetic alleles may be created through gene editing techniques that are known in the art, and may differ genetically (e.g., in their nucleic acid sequence) and/or epigenetically (e.g., in their DNA methylation status, histone acetylation status, chromatin structure, or in other aspects that do not materially alter the coding sequence) from non-synthetic alleles of the same gene in the reference cell from the subject. Synthetic alleles may be modified in their coding and/or non-coding sequence(s). In some aspects, the nucleic acid sequence may be inserted into the genome of the modified cell so that it is under the control of an endogenous promoter in the genome of the modified cell. In some aspects, the nucleic acid sequence may be inserted within, or downstream (i.e., following the 3′end), of a locus in the genome of the modified cell such that the nucleic acid sequence in the inserted nucleic acid sequence is under control of a promoter for the locus. As used herein, “locus” refers to a location within a genome that contains a specific gene, which may contain one or more exons and/or introns. Thus, the terms “locus” and “gene” may be used interchangeably herein. In some aspects, the nucleic acid sequence is inserted within a gene or replaces all or part of a gene. In some aspects, the nucleic acid sequence comprises a nucleic acid sequence that encodes a therapeutic protein, and the nucleic acid sequence is inserted into a locus so that the nucleic acid sequence is in-frame with the coding sequence of an exon within the locus. In some aspects, the nucleic acid sequence is inserted into a locus so that the nucleic acid sequence is in-frame with any exon in the locus that yields the desired outcome. For example, in some aspects, the exon may be the first exon within the locus. In some aspects, the exon may be the last exon in the locus. As used herein, “in-frame with the coding sequence of an exon,” “in-frame with an exon,” and the like, mean that when an exonic sequence and a nucleic acid sequence encoding a protein, such as a therapeutic protein, are joined, the joined sequences form one single open reading frame (ORF). In some aspects, a first polynucleotide encoding a protease cleavage sequence, or a self-cleaving peptide (e.g., a 2A peptide), may be inserted, in-frame, between the exonic sequence and the nucleic acid sequence encoding a therapeutic protein. The resulting encoded protein will contain a protease cleavage sequence, or a self-cleaving peptide, between the amino acid sequence encoded by the exonic sequence and the therapeutic protein, thereby allowing the therapeutic protein to be separated from, or produced independently from, the amino acid sequence encoded by the exonic sequence. In some aspects, a second polynucleotide encoding a secreted peptide signal sequence (SP) may be joined to the nucleic acid sequence encoding a therapeutic protein so that the sequence encoding the signal peptide is in-frame with the therapeutic protein-encoding nucleic acid sequence. In some aspects, a nucleotide sequence encoding one or domains of the therapeutic protein, such as a cytoplasmic domain and/or a transmembrane domain, may be deleted so that the therapeutic protein lacks such domains.

Suitable gene-editing techniques for inserting the nucleic acid sequence into the genome of the modified cell may include any gene-editing system known in the art. In some aspects, suitable gene editing techniques may include conventional genome editing systems, such as conventional homologous recombination, ssODNs homologous recombination; chemical systems, such as peptide NA systems; protein based nuclease systems, such as meganuclease systems, zinc-finger nuclease systems and TALEN systems; homing endonuclease (HE) systems, such as Adeno-Associated Virus (AAA) systems; and RNA-protein based systems, such as CRISPR systems, PRIME editing, and the like.

As used herein, “therapeutic molecule” refers to a molecule that, when administered to, or expressed in, an individual, reduces and/or eliminates and/or ameliorates and/or treats and/or prevents at least one amyloid beta-related pathology phenotype. In some aspects, the therapeutic molecule may treat the primary cause of the disease. For example, the therapeutic molecule may reduce the number of, or the amount of soluble or insoluble β-amyloid (Aβ) peptide oligomers, pyroglutamate Aβ, protofibrils, fibrils, and/or total plaque load. In some embodiments, the therapeutic molecule may enhance amyloid proteolysis. In some embodiments, the therapeutic molecule may enhance microglial phagocytosis of amyloid beta. The present invention is not limited to the mechanism by which the therapeutic molecule affects (e.g., reduces, eliminates, ameliorates, treats, prevents, etc.) the amyloid beta-related pathology phenotype. Any type of therapeutic molecule may be used when producing modified cells of the disclosure. The therapeutic molecule may be a therapeutic RNA molecule, or it may be a therapeutic protein, which may be a membrane-bound protein or a secreted protein. Therapeutic proteins useful for producing modified cells of the disclosure include, but are not limited to, enzymes, peptide or protein binding domains (e.g., antibodies or fragments thereof), nucleic acid binding proteins, chimeric proteins such as chimeric antigen receptors, anti-inflammatory proteins, thrombolytic proteins, immunomodulating molecules, proteases, and metallo-endopeptidases, one example of which is neprilysin. In some aspects, the therapeutic protein is selected from the group consisting of enzymes, peptide or protein binding domains, nucleic acid binding proteins, chimeric proteins such as chimeric antigen receptors, anti-inflammatory proteins, thrombolytic proteins, proteases, and metallo-endopeptidases. In some aspects, the therapeutic protein is a metallo-endopeptidase. In some aspects, the therapeutic protein is neprilysin, which may be a membrane-bound neprilysin or a secreted form of neprilysin.

The therapeutic molecule may be a therapeutic RNA. Therapeutic RNAs useful for producing modified cells of the disclosure include, but are not limited to, inhibitors of mRNA translation (e.g., antisense molecules), molecules that interfere with RNA (e.g., RNAi), catalytically active RNA molecules (e.g., ribozymes) and RNAs that bind proteins and other ligands (e.g., aptamers). Methods of producing such molecules are known to those skilled in the art and are also disclosed in U.S. Patent Publication No. 2014/0303073, U.S. Patent Publication No. 2012/0232128, U.S. Patent Publication No. 2011/0118334, U.S. Patent Publication No. 2011/0033859, U.S. Patent Publication No. 2006/0089323, U.S. Patent Publication No. 2012/0263782, U.S. Patent Publication No. 2012/0301449, and U.S. Patent Publication No. 2004/0137429, the entire disclosures of which are incorporated herein by reference.

In some aspects, a therapeutic molecule of the disclosure may affect (e.g., reduce) at least one amyloid beta-related pathology phenotype. As used herein, the term “Alzheimer's disease-related phenotype” means any observable characteristic or trait of Alzheimer's disease, such as a behavior, which may comprise memory problems, cognitive problems, vision problems, behavioral changes, personality changes, depression, and seizures. In some aspects, the therapeutic molecule may alter (e.g., reduce, eliminate, prevent, ameliorate, etc.) at least one aspect of Alzheimer's disease-related pathology. As used herein, “pathology” refers to anatomic and/or physiologic changes that result from a disease. In Alzheimer's disease, it is thought that β-amyloid (Aβ) protein plays a seminal role in development of the disease. For example, pieces of β-amyloid (Aβ) peptide can clump together to form plaques, which are observed in the brains of Alzheimer's patients. The presence of such Aβ plaques may be considered Alzheimer's disease-related pathology. Similarly, it has been observed that a species of Aβ known as Aβ oligomers (AβOs) are noticeably increased in the early stages of Alzheimer's disease, and localize at or within the synapse. The presence of such AβOs may also be considered Alzheimer's disease-related pathology. Further, another hallmark of Alzheimer's patients is the presence of neurofibrillary tangles, which comprise threads of tau protein. The presence of such neurofibrillary tangles may also be considered Alzheimer's disease-related pathology. Thus, in some aspects, the at least one aspect of Alzheimer's disease-related pathology may comprise formation or presence of Aβ plaques, AβOs, and/or neurofibrillary tangles.

As used herein, the term “amyloid beta-related pathology phenotype” means any observable characteristic or trait of a disease or condition associated with amyloid beta-related pathology, such as a behavior, which may comprise memory problems, cognitive problems, vision problems, behavioral changes, personality changes, depression, and seizures. In some aspects, the therapeutic molecule may alter (e.g., reduce, eliminate, prevent, ameliorate, etc.) at least one aspect of amyloid beta-related pathology.

In certain aspects, the therapeutic molecule is a therapeutic protein. Such protein may have enzymatic and/or anti-inflammatory activity. In some aspects, the therapeutic protein may have protease activity. In some aspects, the therapeutic protein may be a metalloprotease, one example of which is neprilysin. In some aspects, the therapeutic protein may be neprilysin. In some aspects, the therapeutic protein comprises, or consists of, an amino acid sequence at least 70%, at least 75%, at least 80%, at least 85%, at least 90%, at least 95%, at least 97%, or at least 99% identical to SEQ ID NO:1, SEQ ID NO:2, or SEQ ID NO:3, wherein the therapeutic protein has neprilysin activity. In some aspects, the therapeutic protein comprises, or consists of, an amino acid sequence at least 70%, at least 75%, at least 80%, at least 85%, at least 90%, at least 95%, at least 97%, or at least 99% identical to SEQ ID NO:1, SEQ ID NO:2, or SEQ ID NO:3, wherein the therapeutic protein has neprilysin activity, and wherein the differences in sequence are due to conservative amino acid substitutions. In some aspects, the therapeutic protein comprises, or consists of, SEQ ID NO:1, SEQ ID NO:2, or SEQ ID NO:3. In some aspects, the therapeutic protein comprises SEQ ID NO: 1. In some aspects, the therapeutic protein comprises SEQ ID NO: 2. In some aspects, the therapeutic protein comprises SEQ ID NO: 3. In some aspects, the therapeutic protein comprises an amino acid sequence at least 70%, at least 75%, at least 80%, at least 85%, at least 90%, at least 95%, at least 97%, or at least 99% identical to SEQ ID NO:1. In some aspects, the therapeutic protein comprises an amino acid sequence at least 70%, at least 75%, at least 80%, at least 85%, at least 90%, at least 95%, at least 97%, or at least 99% identical to SEQ ID NO: 2. In some aspects, the therapeutic protein comprises an amino acid sequence at least 70%, at least 75%, at least 80%, at least 85%, at least 90%, at least 95%, at least 97%, or at least 99% identical to SEQ ID NO:3.

TABLE 1
Non-limiting examples of therapeutic proteins.

| | | SEQ ID |
|---|---|---|
| | Sequence | NO: |
| Full-length Neprilysin; | MGKSESQMDITDINTPKPKKKQRWTPLEISLSVLVLLLTIIAVTM | 1 |
| includes transmembrane | IALYATYDDGICKSSDCIKSAARLIQNMDATTEPCTDFFKYACG | |
| (Bold) and cytoplasmic | GWLKRNVIPETSSRYGNFDILRDELEVVLKDVLQEPKTEDIVAV | |
| domains (underlined). | QKAKALYRSCINESAIDSRGGEPLLKLLPDIYGWPVATENWEQ | |
| | KYGASWTAEKAIAQLNSKYGKKVLINLFVGTDDKNSVNHVIHID | |
| | QPRLGLPSRDYYECTGIYKEACTAYVDFMISVARLIRQEERLPI | |
| | DENQLALEMNKVMELEKEIANATAKPEDRNDPMLLYNKMTLA | |
| | QIQNNFSLEINGKPFSWLNFTNEIMSTVNISITNEEDVVVYAPE | |
| | YLTKLKPILTKYSARDLQNLMSWRFIMDLVSSLSRTYKESRNAF | |
| | RKALYGTTSETATWRRCANYVNGNMENAVGRLYVEAAFAGES | |
| | KHVVEDLIAQIREVFIQTLDDLTWMDAETKKRAEEKALAIKERIG | |
| | YPDDIVSNDNKLNNEYLELNYKEDEYFENIIQNLKFSQSKQLKK | |
| | LREKVDKDEWISGAAVVNAFYSSGRNQIVFPAGILQPPFFSAQ | |
| | QSNSLNYGGIGMVIGHEITHGFDDNGRNFNKDGDLVDWWTQ | |
| | QSASNFKEQSQCMVYQYGNFSWDLAGGQHLNGINTLGENIA | |
| | DNGGLGQAYRAYQNYIKKNGEEKLLPGLDLNHKQLFFLNFAQ | |
| | VWCGTYRPEYAVNSIKTDVHSPGNFRIIGTLQNSAEFSEAFHC | |
| | RKNSYMNPEKKCRVW* | |
| Truncated neprilysin; | YDDGICKSSDCIKSAARLIQNMDATTEPCTDFFKYACGGWLKR | 2 |
| lacks transmembrane | NVIPETSSRYGNFDILRDELEVVLKDVLQEPKTEDIVAVQKAKA | |
| and cytoplasmic domains | LYRSCINESAIDSRGGEPLLKLLPDIYGWPVATENWEQKYGAS | |
| | WTAEKAIAQLNSKYGKKVLINLFVGTDDKNSVNHVIHIDQPRLG | |
| | LPSRDYYECTGIYKEACTAYVDFMISVARLIRQEERLPIDENQL | |
| | ALEMNKVMELEKEIANATAKPEDRNDPMLLYNKMTLAQIQNNF | |
| | SLEINGKPFSWLNFTNEIMSTVNISITNEEDVVVYAPEYLTKLKP | |
| | ILTKYSARDLQNLMSWRFIMDLVSSLSRTYKESRNAFRKALYG | |
| | TTSETATWRRCANYVNGNMENAVGRLYVEAAFAGESKHVVE | |
| | DLIAQIREVFIQTLDDLTWMDAETKKRAEEKALAIKERIGYPDDI | |
| | VSNDNKLNNEYLELNYKEDEYFENIIQNLKFSQSKQLKKLREK | |
| | VDKDEWISGAAVVNAFYSSGRNQIVFPAGILQPPFFSAQQSNS | |
| | LNYGGIGMVIGHEITHGFDDNGRNFNKDGDLVDWWTQQSAS | |
| | NFKEQSQCMVYQYGNFSWDLAGGQHLNGINTLGENIADNGG | |
| | LGQAYRAYQNYIKKNGEEKLLPGLDLNHKQLFFLNFAQVWCG | |
| | TYRPEYAVNSIKTDVHSPGNFRIIGTLQNSAEFSEAFHCRKNSY | |
| | MNPEKKCRVW* | |
| Secreted neprilysin; | MGAASGRRGPGLLLPLPLLLLLPPQPALAYDDGICKSSDCIK | 3 |
| contains secretion signal | SAARLIQNMDATTEPCTDFFKYACGGWLKRNVIPETSSRYGNF | |
| at N-terminus (Bold) | DILRDELEVVLKDVLQEPKTEDIVAVQKAKALYRSCINESAIDSR | |
| | GGEPLLKLLPDIYGWPVATENWEQKYGASWTAEKAIAQLNSK | |
| | YGKKVLINLFVGTDDKNSVNHVIHIDQPRLGLPSRDYYECTGIY | |
| | KEACTAYVDFMISVARLIRQEERLPIDENQLALEMNKVMELEKE | |
| | IANATAKPEDRNDPMLLYNKMTLAQIQNNFSLEINGKPFSWLN | |
| | FTNEIMSTVNISITNEEDVVVYAPEYLTKLKPILTKYSARDLQNL | |
| | MSWRFIMDLVSSLSRTYKESRNAFRKALYGTTSETATWRRCA | |
| | NYVNGNMENAVGRLYVEAAFAGESKHVVEDLIAQIREVFIQTL | |
| | DDLTWMDAETKKRAEEKALAIKERIGYPDDIVSNDNKLNNEYL | |
| | ELNYKEDEYFENIIQNLKFSQSKQLKKLREKVDKDEWISGAAV | |
| | VNAFYSSGRNQIVFPAGILQPPFFSAQQSNSLNYGGIGMVIGH | |
| | EITHGFDDNGRNFNKDGDLVDWWTQQSASNFKEQSQCMVY | |
| | QYGNFSWDLAGGQHLNGINTLGENIADNGGLGQAYRAYQNYI | |
| | KKNGEEKLLPGLDLNHKQLFFLNFAQVWCGTYRPEYAVNSIKT | |
| | DVHSPGNFRIIGTLQNSAEFSEAFHCRKNSYMNPEKKCRVW* | |

*indicates a STOP codon; As used herein a “secretion signal” and “signal peptide” may be used interchangeably.

In modified cells of the disclosure, the nucleic acid sequence encoding the therapeutic molecule is operably linked to a pathology responsive promoter. For example, the expression of the encoded therapeutic molecule by the operably linked promoter may be induced by the presence of amyloid beta-related pathology. As used herein, “induced by the presence of amyloid beta-related pathology,” “amyloid beta-related pathology responsive,” “pathology-responsive” and the like, mean that expression of the therapeutic molecule occurs when the modified cell is proximate to or in contact with amyloid beta-related pathology. Without being bound by theory, it is believed that activation of the promoter operably linked to the therapeutic molecule-encoding nucleic acid sequence occurs in response to endogenous and/or exogenous signals resulting from contact of the cell with the pathology, or with molecules resulting from the presence of extent of the pathology. One example of such a signal is a signal that induces an “activated” or “disease-associated microglial” phenotype. In some aspects, expression of the encoded therapeutic molecule is dependent on the modified cell being proximate to or in contact with amyloid beta-related pathology. In some aspects, expression of the encoded therapeutic molecule is dependent on the modified cell being in contact with amyloid beta-related pathology. In some aspects, the expression of the encoded therapeutic molecule is dependent on the modified cell being proximate to or in contact with β-amyloid (Aβ) peptide plaques, soluble Aβ monomers, insoluble Aβ monomers, Aβ oligomers, pyroglutamate Aβ, protofibrils, or fibrils comprising Aβ of varying lengths.

In some aspects, the nucleic acid sequence encoding the therapeutic molecule may be operatively linked to a pathology-responsive promoter. As used herein, the term operatively linked refers to two or more nucleic acid sequences, or partial sequences, which are positioned so that they functionally interact to perform their intended functions. For example, a promoter may be functionally linked to a nucleic acid (e.g., coding) molecule if it can control or modulate transcription of a nucleic acid sequence in the cis position in the nucleic acid sequence. Generally, but not necessarily, functionally linked nucleic acid sequences are close together. Although a functionally linked promoter may generally be located upstream of the coding sequence, it does not necessarily have to be close to it. For example, a nucleic acid sequence encoding a therapeutic molecule may be inserted into a gene comprising several exons, placing transcription of the nucleic acid sequence under control of the gene's promoter. If the therapeutic molecule-encoding nucleic acid sequence is joined in, inframe, with the last exon of the gene, the nucleic acid sequence would be under the control of the gene's promoter while potentially being quite distant from it. Enhancers need not be close by either, provided that they assist the transcription of the nucleic acid sequence. For this purpose, they may be both upstream and/or downstream of the nucleic acid sequence, possibly at some distance from it. A polyadenylation site is functionally linked to a polynucleotide sequence if it is positioned at the 3′ end of the sequence in such a way that the transcription progresses via the coding sequence to the polyadenylation signal. Accordingly, two or more nucleic acid sequences that are functionally linked may or may not be in direct contact (i.e., immediately adjacent to one another in the virus vector genome).

As used herein, “amyloid beta-related pathology responsive promoter,” “pathology responsive promoter,” and the like refer to a promoter within a cell that activates transcription of a nucleotide sequence operatively linked to the promoter, when the cell is proximate to or in contact with amyloid beta-related pathology. Thus, a nucleic acid sequence encoding a therapeutic molecule for a disease, and operatively linked to a pathology responsive promoter within a cell, is transcribed when the cell is proximate to or in contact with amyloid beta-related pathology. Any promoter may be considered as a pathology-responsive promoter, as long as transcription of the promoter is activated when a cell comprising the promoter is proximate to, or in contact with, amyloid beta-related pathology. In some aspects, the pathology-responsive promoter is exogenous to the modified cell. In some aspects, the pathology-responsive promoter is endogenous to the modified cell. In some aspects, the therapeutic molecule-encoding nucleic acid sequence is inserted into the genome of the modified cell so that transcription of the therapeutic molecule-encoding sequence is under the control of a pathology-responsive promoter in the genome. Examples of pathology-responsive promoters useful for producing a modified cell of the disclosure include, but are not limited to, a Dendrocyte Expressed Seven Transmembrane Protein (DCSTAMP) gene (Gene ID: 81501; MIM: 605933) promoter, a CD9 (tetraspanin) gene (Gene ID: 928; MIM: 143030) promoter, a CD44 gene (Gene ID: 960; MIM: 107269) promoter, a Galectin3 (LGALS3) gene (Gene ID: 3958; MIM: 153619) promoter, a Secreted Phosphoprotein 1 (SPP1) gene (Gene ID: 81502; MIM: 607106) promoter, a Glycoprotein Nmb (GPNMB) gene (Gene ID: 10457; MIM: 604368) promoter, a Major Histocompatibility Complex, Class II, DR Beta (HLA-DRB1) gene (Gene ID: 3123; MIM:142857) promoter, a Lipoprotein Lipase (LPL) gene (Gene ID: 4023; MIM: 609708) promoter, a Lipase A (LIPA) gene (Gene ID: 3988; MIM: 613497) promoter, a FattyAcid Binding Protein 3 (FABP3) gene (Gene ID: 2170; MIM: 134651) promoter, a Membrane Spanning 4-Domains A6A (MS4A6A) gene (Gene ID: 64231; MIM: 606548) promoter, a C-X-C Motif Chemokine Receptor 4 (CXCR4) gene (Gene ID: 7852; MIM: 162643) promoter, a Chitinase-3-like protein 1 (CH13L1) gene (Gene ID: 1116; MIM: 601525) promoter, an Oxidized low-density lipoprotein receptor 1 (OLR1) gene (Gene ID: 4973; MIM: 602601) promoter, a CD36 gene (Gene ID: 948; MIM: 173510) promoter, a Signaling Lymphocyte Activating Molecule (SLAM) F8 (SLAMF8) gene (Gene ID: 56833; MIM: 606620) promoter, a Triggering Receptor Expressed On Myeloid Cells 2 (TREM2) gene (Gene ID: 54209; MIM: 605086) promoter, a Macrophage Scavenger Receptor 1 (MSR1) gene (Gene ID: 4481; MIM: 153622) promoter, a Beta-2 Microglobulin (B2M) gene (Gene ID: 567; MIM: 109700) promoter, an Integrin Subunit Alpha X (ITGAX) gene (Gene ID:3687; MIM: 151510) promoter, and a Melanocyte Inducing Transcription Factor (MITF) gene (Gene ID: 4286; MIM: 156845) promoter.

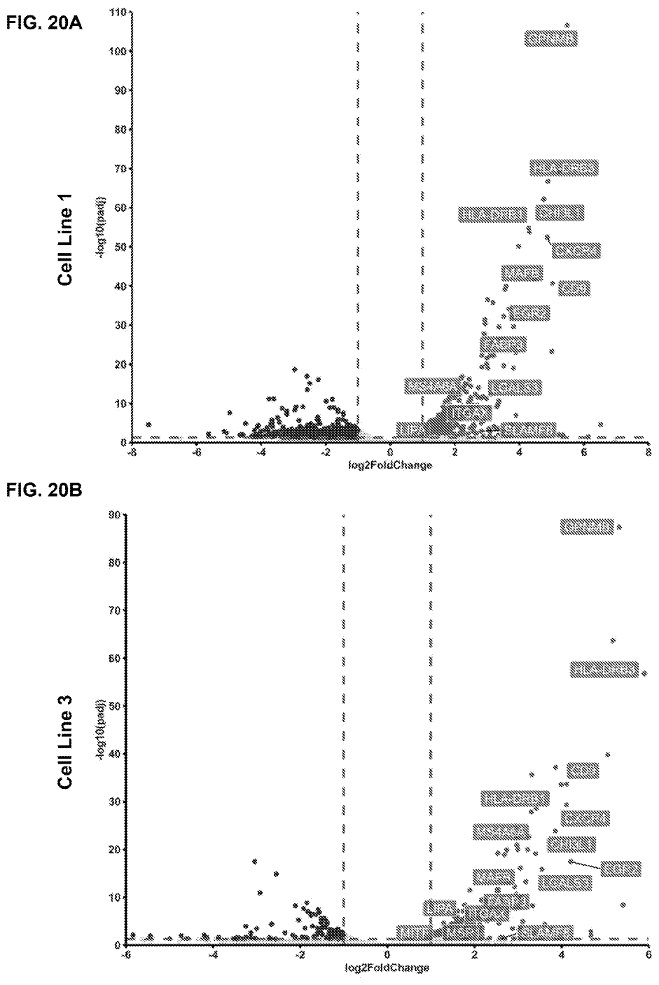

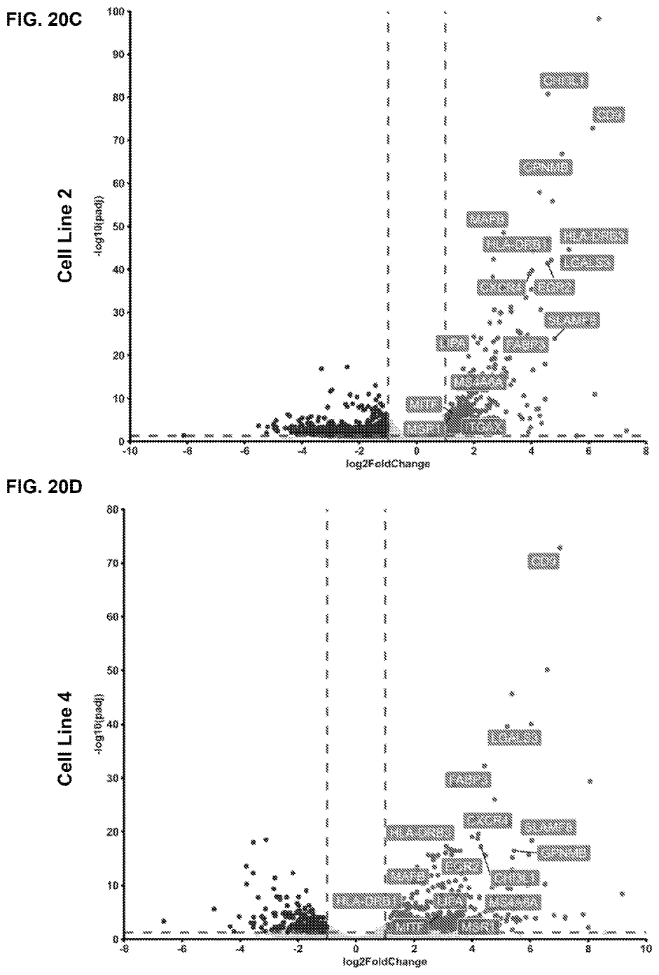

Other non-limiting examples of pathology-responsive promoters useful for producing a modified cell of the disclosure include, but are not limited to, HLA-DRB3, HLA-DRB5, HLA-DRA, CHIT1, DKK2, HLA-DQB1, PLXNA1, CCR7, HLA-DPA1, RGCC, COX6A2, HLA-DRB1, HLA-DPB1, TAFA3, FAIM2, ZNF804A, LGALS1, DOK2, HLA-DOA, GPR153, SLPI, ADGRF5, MYOZ1, HBEGF, BHLHE40, TMEM37, SLC16A8, GRM5, S100A4, GABRB1, LONRF3, PIGR, RAB7B, BIRC7, FABP5, CA2, FCMR, GABRB3, ATF7-NPFF, ANXA2, EGR2, OR9G1, IFNLR1, APOC1, CD200R1, PKD2L1, TDRD6, HLA-DMA, KCNJ5, GLDN, PREX2, OLFM2, PADI2, COL1A2, RRAGD, ROR2, PTCRA, EDN1, RAMP1, BCL2A1, CD83, CHST2, DAGLA, MAFB, DUSP2, FPR3, DPYD, PHLDA1, PTPRG, IER5L, CPNE8, MPDZ, RNF152, PTGER4, MCF2, SDS, CIITA, RGS16, ANGPTL6, PLA2G7, CCL3, ATF3, RAB42, TBX18, STARD13, NR4A3, S100A13, TRDN, GADD45G, ADRA1B, LYPD1, FXYD6, EPHB6, FERMT2, ART4, TNFSF18, TNFSF15, SLC38A4, CDH6, SCIN, FGR, ZNF385B, FUT9, SORCS1, KLF10, CFD, TRPM1, FLVCR2, NFIL3, MYO1E, ITPRID1, SLC47A1, LITAF, RASGEF1B, DENND2D, ADCY3, SYTL3, ARL4C, IL411, CDCP1, TRERF1, VAT1, INPP1, ARRDC4, ENPP1, GSDME, APOC2, COL4A1, CDKN1A, KCNC4, KCNJ2, USP2, UNC13B, VEGFB, TNFAIP2, SLC35F2, HLA-DMB, NAB2, ACP5, CYTL1, FXYD5, ALDH1A2, TMEM176B, TAGLN2, TIMD4, MEP1A, ADGRL2, FAM20C, LRRC39, CD74, RASGRP3, HEG1, TNFRSF14, ADGRE2, FAM110B, GADD45B, TSKU, ID2, ADGRD1, DUSP10, TNFSF13B, FKBP5, MYC, BCL6, TNFAIP8L3, ARID5B, APOC4-APOC2, PMAIP1, P2RY8, CCRL2, RNASE1, LSP1, ARHGEF3, CLEC19A, KCNQ5, DTNA, PILRA, WIPF3, CSTB, XYLT1, DBI, SDC2, KCNMA1, ALCAM, TSPAN4, RPS28, KLHDC8B, MBOAT1, FAM20A, CDH8, NAP1L1, SMIM4, ALAS1, PDLIM7, IQGAP2, INPP4B, TRIM58, SLC36A1, TXN, HLA-B, HSPB1, AP1S2, PLAU, HLA-DQA1, CYSTM1, IER5, MS4A7, SPN, CTSD, SELENOP, SDSL, CD14, HLA-F, B3GALNT1, GAPT, RASSF3, GAS7, GAS2L3, GPD2, CPM, SGPP1, LILRA4, NEB, PRXL2C, SNX24, PPARG, CAPG, CD6, INHBA, PTPN7, LYRM9, ATOX1, UGCG, TMSB10, IFI30, M1AP, SH3BGRL3, CMYA5, PRKCH, NIBAN1, GK, HSD3B7, GPR65, LGALS9, RPL36, SH3RF1, WWC3, ALDH5A1, SNTB2, HACD4, LTA4H, SCIMP, SLC11A1, PTPDC1, CPNE2, CALHM2, PRDX1, APOE, IQGAP1, OSBPL3, GRK3, DPEP2, TMEM154, FOXP1, GYPC, SLC15A3, NENF, HPSE, TNFRSF11A, PPM1M, HPCAL1, SLC46A1, SLC1A3, MORC1, GABARAPL1, MGLL, TGIF1, ZYX, MAP4, or MFSD2A (See FIG. 20A-20D).

In certain aspects, the pathology-responsive promoter comprises a CD9 gene promoter, or a functional variant thereof. As used herein, “functional variant” refers to a promoter having a nucleotide sequence at least 95% identical to a native promoter, and which has at least 50%, at least 60%, at least 70%, at least 80%, at least 90%, at least 95%, at least 97%, or at least 99%, of the activity of the native promoter. The present invention is not limited to the aforementioned pathology-responsive promoters.

Heretofore has been generally described modified cells encoding therapeutic molecules, the expression of which are under the control of pathology-responsive promoters. A detailed embodiment of one such cell will now be described. It should be understood that such description is not intended to limit the disclosure to a particular embodiment but is provided merely to help illustrate one possible embodiment using specific elements of the disclosure.

One aspect of the disclosure is a modified microglial-like (iMGL) cell comprising a nucleic acid sequence encoding a therapeutic molecule, which may be a therapeutic protein, wherein the nucleic acid sequence is operatively linked to a pathology responsive promoter, and wherein the therapeutic protein can reduce a least one amyloid beta-related pathology phenotype. In some aspects, the pathology responsive promoter comprises a promoter from a gene selected from the group consisting of a DCSTAMP gene, a CD9 gene, a CD44 gene, an LGALS3 gene, an SPP1 gene, a GPNMB gene, an HLA-DRB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, an ITGAX gene, and a MITF gene. In some aspects, the nucleic acid sequence may be inserted into a gene selected from the group consisting of a DCSTAMP gene, a CD9 gene, a CD44 gene, an LGALS3 gene, an SPP1 gene, a GPNMB gene, an HLA-DRB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, an ITGAX gene, and a MITF gene. In some aspects, the nucleic acid sequence may be inserted into the genome of the iMGL cell such that the nucleic acid sequence is operatively linked to the endogenous promoter of a gene selected from the group consisting of a DCSTAMP gene, a CD9 gene, a CD44 gene, an LGALS3 gene, an SPP1 gene, a GPNMB gene, an HLA-DRB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, an ITGAX gene, and a MITF gene. In some aspects, the nucleic acid sequence may be inserted into a gene selected from the group consisting of a DCSTAMP gene, a CD9 gene, a CD44 gene, an LGALS3 gene, an SPP1 gene, a GPNMB gene, an HLA-DRB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, an ITGAX gene, and a MITF gene so that the nucleic acid sequence encoding a therapeutic protein is joined to, and in-frame with, an exon of the gene. In some aspects, the nucleic acid sequence encoding the therapeutic protein is joined to and in-frame with the first exon of the gene. In some aspects, the nucleic acid sequence encoding the therapeutic protein is joined to and in-frame with the last exon of the gene. In some aspects, a first polynucleotide encoding a protease cleavage sequence or a self-cleaving peptide may be inserted between the exon and the nucleic acid sequence encoding the therapeutic protein, such that the first polynucleotide is in-frame with the exon and the nucleic acid sequence encoding the therapeutic protein. In some aspects, a second polynucleotide encoding a secreted peptide signal sequence (SP) may be joined to the nucleic acid sequence encoding the therapeutic protein, wherein the second polynucleotide may be inserted between the first polynucleotide and the therapeutic protein-encoding nucleic acid sequence, such that the encoded signal peptide is in-frame with the exon and the therapeutic protein-encoding nucleic acid sequence. In some aspects, the therapeutic protein may be a metalloprotease. In some aspects, the therapeutic protein may be neprilysin, which may be a secreted form of neprilysin or a membrane-bound form of neprilysin. In some aspects, the therapeutic protein may comprise, or consist of, an amino acid sequence at least 70%, at least 75%, at least 80%, at least 85%, at least 90%, at least 95%, at least 97%, or at least 99% identical to SEQ ID NO:1, SEQ ID NO:2, or SEQ ID NO:3, wherein the encoded therapeutic protein has neprilysin activity. In some aspects, the encoded therapeutic protein may comprise, or consist of, an amino acid sequence at least 85%, at least 90%, at least 95%, at least 97%, or at least 99% identical to SEQ ID NO:1, SEQ ID NO:2, or SEQ ID NO:3, wherein the encoded therapeutic protein has neprilysin activity, and wherein the difference between the amino acid sequence of the therapeutic protein and SEQ ID NO:1, SEQ ID NO:2, or SEQ ID NO:3 is due to conservative amino acid substitutions. In some aspects, the encoded protein may comprise, or consist of, SEQ ID NO:1, SEQ ID NO:2, or SEQ ID NO:3.

One aspect of the disclosure is a modified microglial-like (iMGL) cell comprising a nucleic acid sequence that comprises a nucleic acid sequence encoding a therapeutic protein, wherein the nucleic acid sequence is operatively linked to a pathology responsive promoter, and wherein the therapeutic protein can reduce a least one amyloid beta-related pathology. In some aspects, the pathology responsive promoter is a CD9 gene promoter. In some aspects, the nucleic acid sequence may be inserted into the genome of the iMGL cell such that the nucleic acid sequence is operatively linked to the endogenous CD9 promoter. In some aspects, the nucleic acid sequence may be inserted into the CD9 locus. In some aspects, the nucleic acid sequence may be inserted into the CD9 locus so that the nucleic acid sequence encoding the therapeutic protein is joined to, and in-frame with, an exon of the CD9 gene. In some aspects, the nucleic acid sequence encoding the therapeutic protein is joined to and in-frame with the first exon of the CD9 gene. In some aspects, the nucleic acid sequence encoding the therapeutic protein is joined to and in-frame with the last exon of the CD9 gene. In some aspects, a first polynucleotide encoding a protease cleavage sequence or a self-cleaving peptide may be inserted between the CD9 exon and the nucleic acid sequence encoding the therapeutic protein, such that the first polynucleotide is in-frame with the CD9 exon and the nucleic acid sequence encoding the therapeutic protein. In some aspects, a second polynucleotide encoding a secreted peptide signal sequence (SP) may be joined to the nucleic acid sequence encoding the therapeutic protein, wherein the second polynucleotide may be inserted between the first polynucleotide and the therapeutic protein-encoding nucleic acid sequence, such that the encoded signal peptide is in-frame with the CD9 exon and the therapeutic protein-encoding nucleic acid sequence. In some aspects, the therapeutic protein may be a metalloprotease. In some aspects, the therapeutic protein may be neprilysin, which may be a secreted form of neprilysin or a membrane-bound form of neprilysin. In some aspects, the therapeutic protein may comprise, or consist of, an amino acid sequence at least 70%, at least 75%, at least 80%, at least 85%, at least 90%, at least 95%, at least 97%, or at least 99% identical to SEQ ID NO:1, SEQ ID NO:2, or SEQ ID NO:3, wherein the encoded therapeutic protein has neprilysin activity. In some aspects, the encoded therapeutic protein may comprise, or consist of, an amino acid sequence at least 85%, at least 90%, at least 95%, at least 97%, or at least 99% identical to SEQ ID NO:1, SEQ ID NO:2, or SEQ ID NO:3, wherein the encoded therapeutic protein has neprilysin activity, and wherein the difference between the amino acid sequence of the therapeutic protein and SEQ ID NO:1, SEQ ID NO:2, or SEQ ID NO:3 is due to conservative amino acid substitutions. In some aspects, the encoded protein may comprise, or consist of, SEQ ID NO:1, SEQ ID NO:2, or SEQ ID NO:3. In some aspects, the encoded therapeutic protein comprises SEQ ID NO: 1. In some aspects, the encoded therapeutic protein comprises SEQ ID NO: 2. In some aspects, the encoded therapeutic protein comprises SEQ ID NO: 3. In some aspects, the encoded therapeutic protein comprises an amino acid sequence at least 70%, at least 75%, at least 80%, at least 85%, at least 90%, at least 95%, at least 97%, or at least 99% identical to SEQ ID NO:1. In some aspects, the encoded therapeutic protein comprises an amino acid sequence at least 70%, at least 75%, at least 80%, at least 85%, at least 90%, at least 95%, at least 97%, or at least 99% identical to SEQ ID NO: 2. In some aspects, the encoded therapeutic protein comprises an amino acid sequence at least 70%, at least 75%, at least 80%, at least 85%, at least 90%, at least 95%, at least 97%, or at least 99% identical to SEQ ID NO:3.

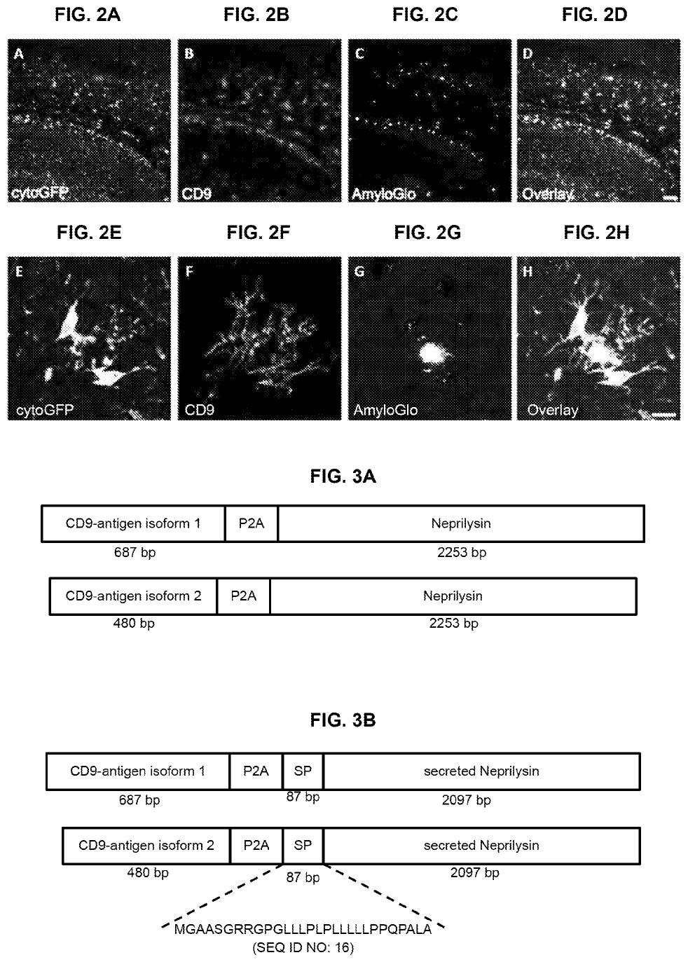

One aspect of the disclosure is a modified microglial-like (MGL) cell comprising: a nucleic acid sequence encoding a therapeutic protein, wherein the nucleic acid sequence is inserted into the CD9 locus of the cell's genome so that the therapeutic protein-encoding nucleic acid sequence is in-frame with at least part of the coding sequence of an exon of the CD9 gene; and, a first polynucleotide encoding a protease cleavage sequence or a self-cleaving peptide inserted between the at least part of the coding sequence of the exon of the CD9 gene and the therapeutic protein-encoding nucleic acid sequence such that the encoded protease cleavage site or self-cleaving peptide is in-frame with the at least part of the coding sequence of the exon of the CD9 gene and the therapeutic protein-encoding nucleic acid sequence; wherein transcription from the CD9 promoter results in a an mRNA encoding a hybrid protein comprising the amino acid sequence encoded by the at least part of the coding sequence of the first exon of the CD9 gene, the protease-cleavage sequence or self-cleaving peptide, and the therapeutic protein. In some aspects, the carboxyl end of at least a part of the encoded CD9 amino acid sequence is joined to the amino-terminal end of the protease-cleavage sequence or self-cleaving peptide sequence. In some aspects, the carboxy-terminal end of the protease-cleavage sequence or self-cleaving peptide sequence is joined to the amino-terminal end of the therapeutic protein. In some aspects, the modified MGL cell comprises a second polynucleotide encoding a secreted peptide signal sequence (SP) joined to the therapeutic protein-encoding nucleic acid sequence, wherein the encoded signal peptide is in-frame with the therapeutic protein-encoding nucleic acid sequence. In some aspects, the modified MGL cell comprises a second polynucleotide encoding a secreted peptide signal sequence (SP) between the first polynucleotide and the therapeutic protein-encoding nucleic acid sequence, wherein the encoded signal peptide is in-frame with the therapeutic protein-encoding nucleic acid sequence. Examples of such aspects are shown in FIGS. 2A and 2B. In some aspects, the therapeutic protein may be a metalloprotease. In some aspects, the therapeutic protein may be neprilysin, which may be a secreted form of neprilysin or a membrane-bound form of neprilysin. In some aspects, the therapeutic protein may comprise, or consist of, an amino acid sequence at least 70%, at least 75%, at least 80%, at least 85%, at least 90%, at least 95%, at least 97%, or at least 99% identical to SEQ ID NO:1, SEQ ID NO:2, or SEQ ID NO:3, wherein the encoded therapeutic protein has neprilysin activity. In some aspects, the encoded therapeutic protein may comprise, or consist of, an amino acid sequence at least 85%, at least 90%, at least 95%, at least 97%, or at least 99% identical to SEQ ID NO:1, SEQ ID NO:2, or SEQ ID NO:3, wherein the encoded therapeutic protein has neprilysin activity, and wherein the difference between the amino acid sequence of the therapeutic protein and SEQ ID NO:1, SEQ ID NO:2, or SEQ ID NO:3 is due to conservative amino acid substitutions. In some aspects, the encoded protein may comprise, or consist of, SEQ ID NO:1, SEQ ID NO:2, or SEQ ID NO:3. In some aspects, the encoded protein comprises SEQ ID NO: 1. In some aspects, the encoded therapeutic protein comprises SEQ ID NO: 2. In some aspects, the encoded therapeutic protein comprises SEQ ID NO: 3. In some aspects, the encoded therapeutic protein comprises an amino acid sequence at least 70%, at least 75%, at least 80%, at least 85%, at least 90%, at least 95%, at least 97%, or at least 99% identical to SEQ ID NO:1. In some aspects, the encoded therapeutic protein comprises an amino acid sequence at least 70%, at least 75%, at least 80%, at least 85%, at least 90%, at least 95%, at least 97%, or at least 99% identical to SEQ ID NO: 2. In some aspects, the encoded therapeutic protein comprises an amino acid sequence at least 70%, at least 75%, at least 80%, at least 85%, at least 90%, at least 95%, at least 97%, or at least 99% identical to SEQ ID NO:3.

One aspect of the disclosure is a therapeutic composition comprising an effective number of modified cells of the disclosure. Such composition may be formulated for administration to an individual having a disease that may be treated by a therapeutic protein expressed by modified cells in the composition. In some aspects, the compositions of the disclosure may be used to treat and/or ameliorate and/or prevent diseases associated with amyloid beta-related pathology.

In some aspects, a composition of the disclosure may comprise a modified cell comprising a nucleic acid sequence that comprises a nucleic acid sequence encoding a therapeutic protein, wherein the nucleic acid sequence is operatively linked to a pathology responsive promoter, and wherein the therapeutic protein can reduce a least one amyloid beta-related pathology phenotype. In some aspects, the nucleic acid sequence may be inserted into a gene selected from the group consisting of a DCSTAMP gene, a CD9 gene, a CD44 gene, an LGALS3 gene, an SPP1 gene, a GPNMB gene, an HLA-DRB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, an ITGAX gene, and a MITF gene. In some aspects, the nucleic acid sequence may be inserted into the genome of the iMGL cell such that the nucleic acid sequence is operatively linked to the endogenous promoter of a gene selected from the group consisting of a DCSTAMP gene, a CD9 gene, a CD44 gene, an LGALS3 gene, an SPP1 gene, a GPNMB gene, an HLA-DRB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, an ITGAX gene, and a MITF gene. In some aspects, the nucleic acid sequence may be inserted into a gene selected from the group consisting of a DCSTAMP gene, a CD9 gene, a CD44 gene, an LGALS3 gene, an SPP1 gene, a GPNMB gene, an HLA-DRB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, an ITGAX gene, and a MITF gene so that the nucleic acid sequence encoding the therapeutic protein is joined to, and in-frame with, an exon of the gene. In some aspects, the nucleic acid sequence encoding the therapeutic protein is joined to and in-frame with the first exon of the gene. In some aspects, the nucleic acid sequence encoding the therapeutic protein is joined to and in-frame with the last exon of the gene. In some aspects, a first polynucleotide encoding a protease cleavage sequence or a self-cleaving peptide may be inserted between the exon and the nucleic acid sequence encoding the therapeutic protein, such that the first polynucleotide is in-frame with the exon and the nucleic acid sequence encoding the therapeutic protein. In some aspects, a second polynucleotide encoding a secreted peptide signal sequence (SP) may be joined to the nucleic acid sequence encoding the therapeutic protein, wherein the second polynucleotide may be inserted between the first polynucleotide and the therapeutic protein-encoding nucleic acid sequence, such that the encoded signal peptide is in-frame with the exon and the therapeutic protein-encoding nucleic acid sequence.

In some aspects, the Alzheimer's disease-related pathology responsive promoter is a CD9 gene promoter. In some aspects, the nucleic acid sequence may be inserted into the genome of the iMGL cell such that the nucleic acid sequence is operatively linked to the endogenous CD9 promoter. In some aspects, the nucleic acid sequence may be inserted into the CD9 locus. In some aspects, the nucleic acid sequence may be inserted into the CD9 locus so that the nucleic acid sequence encoding the therapeutic protein is joined to, and in-frame with, an exon of the CD9 gene. In some aspects, the nucleic acid sequence encoding the therapeutic protein is joined to and in-frame with the first exon of the CD9 gene. In some aspects, the nucleic acid sequence encoding the therapeutic protein is joined to and in-frame with the last exon of the CD9 gene. In some aspects, a first polynucleotide encoding a protease cleavage sequence or a self-cleaving peptide may be inserted between the CD9 exon and the nucleic acid sequence encoding the therapeutic protein, such that the first polynucleotide is in-frame with the CD9 exon and the nucleic acid sequence encoding the therapeutic protein. In some aspects, a second polynucleotide encoding a secreted peptide signal sequence (SP) may be joined to the nucleic acid sequence encoding the therapeutic protein, wherein the second polynucleotide may be inserted between the first polynucleotide and the therapeutic protein-encoding nucleic acid sequence, such that the encoded signal peptide is in-frame with the CD9 exon and the therapeutic protein-encoding nucleic acid sequence. In some aspects, the therapeutic protein may be a metalloprotease. In some aspects, the therapeutic protein may be neprilysin, which may be a secreted form of neprilysin or a membrane-bound form of neprilysin. In some aspects, the therapeutic protein may comprise, or consist of, an amino acid sequence at least 70%, at least 75%, at least 80%, at least 85%, at least 90%, at least 95%, at least 97%, or at least 99% identical to SEQ ID NO:1, SEQ ID NO:2, or SEQ ID NO:3, wherein the encoded therapeutic protein has neprilysin activity. In some aspects, the encoded therapeutic protein may comprise, or consist of, an amino acid sequence at least 85%, at least 90%, at least 95%, at least 97%, or at least 99% identical to SEQ ID NO:1, SEQ ID NO:2, or SEQ ID NO:3, wherein the encoded therapeutic protein has neprilysin activity, and wherein the difference between the amino acid sequence of the therapeutic protein and SEQ ID NO:1, SEQ ID NO:2, or SEQ ID NO:3 is due to conservative amino acid substitutions. In some aspects, the encoded protein may comprise, or consist of, SEQ ID NO:1, SEQ ID NO:2, or SEQ ID NO:3. In some aspects, the encoded therapeutic protein comprises SEQ ID NO: 1. In some aspects, the encoded therapeutic protein comprises SEQ ID NO: 2. In some aspects, the encoded therapeutic protein comprises SEQ ID NO: 3. In some aspects, the encoded therapeutic protein comprises an amino acid sequence at least 70%, at least 75%, at least 80%, at least 85%, at least 90%, at least 95%, at least 97%, or at least 99% identical to SEQ ID NO:1. In some aspects, the encoded therapeutic protein comprises an amino acid sequence at least 70%, at least 75%, at least 80%, at least 85%, at least 90%, at least 95%, at least 97%, or at least 99% identical to SEQ ID NO: 2. In some aspects, the encoded therapeutic protein comprises an amino acid sequence at least 70%, at least 75%, at least 80%, at least 85%, at least 90%, at least 95%, at least 97%, or at least 99% identical to SEQ ID NO:3.

Therapeutic compositions, according to the disclosure, may be administered using any route suitable to the disease being treated. Examples of routes of administration that may be used include but are not limited to, oral administration, parenteral administration, intravascular administration, intravenous administration, intramuscular administration, stereotactic administration, intracerebral administration, intracranial administration, intracerebroventricular administration, and intradermal administration. In certain aspects, it may be advantageous to apply the pharmaceutical compositions as described above via intravenous injection or by direct injection into the target tissue(s). For example, for systemic application, the intravenous, intravascular, intramuscular, stereotactic, intraparenchymal, or intracerebroventricular, may be preferred. A more local application may be affected subcutaneously, intradermally, intracutaneously, intralobally, intramedullarly, or directly in or near the tissue to be treated. Depending on the desired duration and effectiveness of the treatment, the compositions, according to the disclosure, may be administered once or several times, also intermittently, for instance, on a daily basis for several days, weeks or months, and in different dosages.

In various embodiments, the therapeutic composition described herein is administered on a monthly dosage schedule. In other embodiments, the therapeutic composition is administered biweekly. In yet other embodiments, the therapeutic compositions are administered weekly. In certain embodiments, the therapeutic compositions are administered daily. In select embodiments, the therapeutic compositions are administered twice a day. In certain embodiments, the therapeutic composition is administered for at least 3 months, at least 6 months, at least 12 months, or more. In some embodiments, the therapeutic composition is administered for at least 18 months, 2 years, 3 years, or more.

In some aspects, the target tissue(s) comprise brain tissue. In some aspects, the target tissue(s) comprise one or more target brain regions. In some aspects, the target tissue(s) comprise at least 2, 3, 4, 5, 6, 7, 8, 9, 10, or more target brain regions.

In some aspects, the target brain region comprises the cerebral cortex. In some aspects, the target brain region comprises one or more subregions of the cerebral cortex. In some aspects, the target brain region comprises a medial prefrontal cortex or subregion thereof. In some aspects, the target brain region comprises an anterior cingulate cortex or subregion thereof. In some aspects, the target brain region comprises a primary sensory cortex or sensory association cortex.

In some aspects, the target brain region comprises a cerebral ventricle. In some aspects, the cerebral ventricle comprises a lateral ventricle, a third ventricle, a fourth ventricle.

In some aspects, the target brain region comprises a hippocampus or subregion thereof. In some aspects, the target brain region comprises a CA1 region of the hippocampus. In some aspects, the target brain region comprises a CA3 region of the hippocampus. In some aspects, the target brain region comprises a dentate gyrus of the hippocampus. In some aspects, the target brain region comprises a CA2 region of the hippocampus. In some aspects, the target brain region comprises a septal region or fornix of the hippocampus. In some aspects, the target brain region comprises an entorhinal cortex. In some aspects, the target brain region comprises an amygdala or subregion thereof.

In some aspects, the target brain region comprises basal ganglia. In some aspects, the target brain region comprises a basal ganglia nucleus. In some aspects, the basal ganglia nucleus comprises a globus pallidus. In some aspects, the basal ganglia nucleus comprises a substantia nigra pars compacta or substantia nigra pars reticulata. In some aspects, the basal ganglia nucleus comprises a striatum. In some aspects, the basal ganglia nucleus comprises a caudate putamen. In some aspects, the basal ganglia nucleus comprises a subthalamic nucleus.

The administration modes, dosages, and optimum formulations may be determined according to criteria generally taken into account in the establishment of a treatment adapted to an individual such as, for example, the age or the weight, the seriousness of its general condition, the tolerance to the treatment and the secondary effects noted.

Formulations designed for injection into body fluid systems require proper isotonicity and pH buffering to the corresponding levels of body fluids. Isotonicity can be appropriately adjusted with sodium chloride and other salts as needed. Suitable solvents may be used to increase the solubility of the ingredients in the formulation and the stability of the liquid preparation. Further additives that may be employed in the present compositions include but are not limited to, dextrose, conventional antioxidants, and conventional chelating agents. Parenteral dosage forms must also be sterilized prior to use.

One aspect is a method of treating Alzheimer's in an individual, comprising administering to the individual a modified cell of the disclosure or a composition comprising a modified cell of the disclosure. As used herein, “treating Alzheimer's disease,” “treating Alzheimer's,” and the like means reducing the frequency or severity of at least one Alzheimer's disease-related phenotype and/or at least one aspect of an Alzheimer's disease-related pathology. In some aspects, treating Alzheimer's in an individual comprises reducing the incidence or severity of at least one observable characteristic or trait selected from the group consisting of memory problems, learning deficits, cognitive problems, vision problems, behavioral changes, personality changes, depression, and seizures. In some aspects, treating Alzheimer's in an individual may comprise reducing the amount of Aβ peptide in the brain of the individual. In some aspects, treating Alzheimer's in an individual may comprise reducing the size or number of Aβ peptide plaques or soluble or insoluble Aβ protein, oligomers, pyroglutamate Aβ, protofibrils, or fibrils in the brain of the individual, or the amount of Aβ peptide in Aβ peptide plaques in the brain of the individual.

One aspect is a method of treating a disease or condition associated with amyloid beta-related pathology in an individual, comprising administering to the individual a modified cell of the disclosure or a composition comprising a modified cell of the disclosure. As used herein, “treating a disease or condition associated with amyloid beta-related pathology,” “treating amyloid beta-related pathology” and the like means reducing the frequency or severity of at least one amyloid beta-related pathology phenotype and/or at least one aspect of an amyloid beta-related pathology. In some aspects, treating a disease or condition associated with amyloid beta-related pathology in an individual comprises reducing the incidence or severity of at least one observable characteristic or trait selected from the group consisting of memory problems, learning deficits, cognitive problems, vision problems, behavioral changes, personality changes, depression, and seizures. In some aspects, treating a disease or condition associated with amyloid beta-related pathology in an individual may comprise reducing the amount of Aβ peptide in the brain of the individual. In some aspects, treating a disease or condition associated with amyloid beta-related pathology in an individual may comprise reducing the size or number of Aβ peptide plaques or soluble or insoluble Aβ protein, oligomers, pyroglutamate Aβ, protofibrils, or fibrils in the brain of the individual, or the amount of Aβ peptide in Aβ peptide plaques in the brain of the individual.

One aspect is a method of treating a disease or condition associated with amyloid beta-related pathology in an individual comprising administering to the individual a modified cell comprising a nucleic acid sequence encoding a therapeutic protein, wherein the nucleic acid sequence is operatively linked to a pathology responsive promoter, and wherein the therapeutic protein can reduce a least one amyloid beta-related pathology phenotype or pathology. In some aspects, the pathology responsive promoter comprises a promoter from a gene selected from the group consisting of a DCSTAMP gene, a CD9 gene, a CD44 gene, an LGALS3 gene, an SPP1 gene, a GPNMB gene, an HLA-DRB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, an ITGAX gene, and a MITF gene. For example, in some embodiments, the promoter comprises the promoter of a CD9 gene. In some embodiments, the promoter comprises the promoter of an LGALS3 gene. In some embodiments, the promoter comprises the promoter of a HLA-DRB gene. In some embodiments, the promoter comprises the promoter of the CD11c (ITGAX) gene. In some embodiments, the promoter comprises the promoter of a gene selected from: CD9, LGALS3, HLA-DRB, and CD11c. In some embodiments, the promoter comprises the promoter of a gene selected from: CD9, LGALS3, HLA-DRB, TREM2, and CD11c. In some embodiments, the promoter comprises the promoter of a DCSTAMP gene. In some embodiments, the promoter comprises the promoter of a CD44 gene. In some embodiments, the promoter comprises the promoter of an SPP1 gene. In some embodiments, the promoter comprises the promoter of a GPNMB gene. In some embodiments, the promoter comprises the promoter of an LPL gene. In some embodiments, the promoter comprises the promoter of a LIPA gene. In some embodiments, the promoter comprises the promoter of a FABP3 gene. In some embodiments, the promoter comprises the promoter of an MS4A6A gene. In some embodiments, the promoter comprises the promoter of a CXCR4 gene. In some embodiments, the promoter comprises the promoter of a CH13L1 gene. In some embodiments, the promoter comprises the promoter of an OLR1 gene. In some embodiments, the promoter comprises the promoter of a CD36 gene. In some embodiments, the promoter comprises the promoter of a SLAMF8 gene. In some embodiments, the promoter comprises the promoter of a TREM2 gene. In some embodiments, the promoter comprises the promoter of an MSR1 gene. In some embodiments, the promoter comprises the promoter of a B2M gene. In some embodiments, the promoter comprises the promoter of a MITF gene. As previously discussed, the present invention is not limited to the aforementioned promoters.

In some aspects, the nucleic acid sequence may be inserted into a gene selected from the group consisting of a DCSTAMP gene, a CD9 gene, a CD44 gene, an LGALS3 gene, an SPP1 gene, a GPNMB gene, an HLA-DRB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, an ITGAX gene, and a MITF gene. In some aspects, the nucleic acid sequence may be inserted into the genome of the iMGL cell such that the nucleic acid sequence is operatively linked to the endogenous promoter of a gene selected from the group consisting of a DCSTAMP gene, a CD9 gene, a CD44 gene, an LGALS3 gene, an SPP1 gene, a GPNMB gene, an HLA-DRB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, an ITGAX gene, and a MITF gene. In some aspects, the nucleic acid sequence may be inserted into a gene selected from the group consisting of a DCSTAMP gene, a CD9 gene, a CD44 gene, an LGALS3 gene, an SPP1 gene, a GPNMB gene, an HLA-DRB gene, an LPL gene, a LIPA, a FABP3 gene, an MS4A6A gene, a CXCR4 gene, a CH13L1 gene, an OLR1 gene, a CD36 gene, a SLAMF8 gene, a TREM2 gene, an MSR1 gene, a B2M gene, an ITGAX gene, and a MITF gene so that the nucleic acid sequence encoding the therapeutic protein is joined to, and in-frame with, an exon of the gene. In some aspects, the nucleic acid sequence encoding the therapeutic protein is joined to and in-frame with the first exon of the gene. In some aspects, the nucleic acid sequence encoding the therapeutic protein is joined to and in-frame with the last exon of the gene. In some aspects, a first polynucleotide encoding a protease cleavage sequence or a self-cleaving peptide may be inserted between the exon and the nucleic acid sequence encoding the therapeutic protein, such that the first polynucleotide is in-frame with the exon and the nucleic acid sequence encoding the therapeutic protein. In some aspects, a second polynucleotide encoding a secreted peptide signal sequence (SP) may be joined to the nucleic acid sequence encoding the therapeutic protein, wherein the second polynucleotide may be inserted between the first polynucleotide and the therapeutic protein-encoding nucleic acid sequence, such that the encoded signal peptide is in-frame with the exon and the therapeutic protein-encoding nucleic acid sequence. In some aspects, the Alzheimer's disease-related pathology responsive promoter is a CD9 gene promoter. In some aspects, the nucleic acid sequence may be inserted into the genome of the iMGL cell such that the nucleic acid sequence is operatively linked to the endogenous CD9 promoter. In some aspects, the nucleic acid sequence may be inserted into the CD9 locus. In some aspects, the nucleic acid sequence may be inserted into the CD9 locus so that the nucleic acid sequence encoding the therapeutic protein is joined to, and in-frame with, an exon of the CD9 gene. In some aspects, the nucleic acid sequence encoding the therapeutic protein is joined to and in-frame with the first exon of the CD9 gene. In some aspects, the nucleic acid sequence encoding the therapeutic protein is joined to and in-frame with the last exon of the CD9 gene. In some aspects, a first polynucleotide encoding a protease cleavage sequence or a self-cleaving peptide may be inserted between the CD9 exon and the nucleic acid sequence encoding the therapeutic protein, such that the first polynucleotide is in-frame with the CD9 exon and the nucleic acid sequence encoding the therapeutic protein. In some aspects, a second polynucleotide encoding a secreted peptide signal sequence (SP) may be joined to the nucleic acid sequence encoding the therapeutic protein, wherein the second polynucleotide may be inserted between the first polynucleotide and the therapeutic protein-encoding nucleic acid sequence, such that the encoded signal peptide is in-frame with the CD9 exon and the therapeutic protein-encoding nucleic acid sequence. In some aspects, the therapeutic protein may be a metalloprotease. In some aspects, the therapeutic protein may be neprilysin, which may be a secreted form of neprilysin or a membrane-bound form of neprilysin. In some aspects, the therapeutic protein may comprise, or consist of, an amino acid sequence at least 70%, at least 75%, at least 80%, at least 85%, at least 90%, at least 95%, at least 97%, or at least 99% identical to SEQ ID NO:1, SEQ ID NO:2, or SEQ ID NO:3, wherein the encoded therapeutic protein has neprilysin activity. In some aspects, the encoded therapeutic protein may comprise, or consist of, an amino acid sequence at least 85%, at least 90%, at least 95%, at least 97%, or at least 99% identical to SEQ ID NO:1, SEQ ID NO:2, or SEQ ID NO:3, wherein the encoded therapeutic protein has neprilysin activity, and wherein the difference between the amino acid sequence of the therapeutic protein and SEQ ID NO:1, SEQ ID NO:2, or SEQ ID NO:3 is due to conservative amino acid substitutions. In some aspects, the encoded protein may comprise, or consist of, SEQ ID NO:1, SEQ ID NO:2, or SEQ ID NO:3. In some aspects, the therapeutic protein comprises SEQ ID NO: 1. In some aspects, the therapeutic protein comprises SEQ ID NO: 2. In some aspects, the therapeutic protein comprises SEQ ID NO: 3. In some aspects, the therapeutic protein comprises an amino acid sequence at least 70%, at least 75%, at least 80%, at least 85%, at least 90%, at least 95%, at least 97%, or at least 99% identical to SEQ ID NO:1. In some aspects, the therapeutic protein comprises an amino acid sequence at least 70%, at least 75%, at least 80%, at least 85%, at least 90%, at least 95%, at least 97%, or at least 99% identical to SEQ ID NO: 2. In some aspects, the therapeutic protein comprises an amino acid sequence at least 70%, at least 75%, at least 80%, at least 85%, at least 90%, at least 95%, at least 97%, or at least 99% identical to SEQ ID NO:3

One aspect of the disclosure is a kit comprising a modified cell of the disclosure, wherein the modified cell comprises a nucleic acid sequence encoding a therapeutic protein, wherein the nucleic acid sequence is operatively linked to a pathology responsive promoter, and wherein the therapeutic protein can reduce a least one amyloid beta-related pathology phenotype. Kits may also comprise additional components, such as, but not limited to, buffers, labels, containers, tubing, vials, syringes, instructions for administering a modified cell of the disclosure, and the like.

The present invention may include methods of treating neurodegenerative diseases, disorders, or conditions associated with amyloid beta pathology, including but not limited to Alzheimer's disease, and alleviating associated symptoms or pathological processes. These methods may comprise administering a therapeutic composition comprising a sufficient number of modified cells of the disclosure, or a modified cell as described herein, to an individual in need of such treatment. The modified cells and/or therapeutic composition can be delivered directly to the brain or at least one specific target brain region of the individual. In some embodiments, the target brain region may include the cerebral cortex or subregions thereof, hippocampus or subregions thereof, basal ganglia or basal ganglia nucleus thereof, cerebral ventricle, or a combination thereof. Furthermore, the target brain region may include specific subregions such as the anterior cingulate cortex, entorhinal cortex, dentate gyrus, CA1, CA3, or CA2 regions of the hippocampus, fornix, primary sensory cortex, sensory association cortex, septum, globus pallidus, substantia nigra pars compacta, substantia nigra pars reticulata, striatum, caudate putamen, or subthalamic nucleus.

In some embodiments, the modified cells and/or therapeutic composition may be administered after the appearance of Aβ disease pathology. In certain embodiments, the administration may occur after the detection of β-amyloid (Aβ) peptide plaques, soluble or insoluble Aβ monomers, Aβ oligomers, pyroglutamate Aβ, protofibrils, or fibrils comprising Aβ or a fragment thereof in the individual. Administration of the modified cells and/or therapeutic composition may be done through stereotactic injection directly into the brain of the individual.

The present invention may also include methods for preventing or attenuating the onset of neurodegenerative disorders or conditions associated with amyloid beta pathology, such as but not limited to Alzheimer's disease. These methods may comprise administering a therapeutic composition comprising a sufficient number of modified cells of the disclosure, or a modified cell as described herein, to an individual in need of such treatment. The modified cells and/or therapeutic composition can be delivered directly to the brain or at least one specific target brain region of the individual. In some embodiments, the target brain region may include the cerebral cortex or subregions thereof, hippocampus or subregions thereof, cerebral ventricle, or a combination thereof. Furthermore, the target brain region may include specific regions such as the anterior cingulate cortex, entorhinal cortex, dentate gyrus, CA1, CA3, or CA2 regions of the hippocampus, fornix, primary sensory cortex, sensory association cortex, septum, globus pallidus, substantia nigra pars compacta, substantia nigra pars reticulata, striatum, caudate putamen, or subthalamic nucleus.

To effectively prevent or delay the onset of neurodegenerative disease pathology, the modified cells and/or therapeutic composition may be administered before the appearance of Aβ disease pathology. In certain embodiments, the administration may occur before the detection of β-amyloid (Aβ) peptide plaques, soluble or insoluble Aβ monomers, Aβ oligomers, pyroglutamate Aβ, protofibrils, or fibrils comprising Aβ or a fragment thereof in the individual. Administration of the modified cells and/or therapeutic composition may be done through stereotactic injection directly into the brain of the individual and may occur prior to adulthood in some embodiments.

Individuals at risk of developing neurodegenerative disease or condition, or those with a genetic mutation associated with a neurodegenerative disorder, may benefit from the methods disclosed herein. Thus, in some embodiments, the individual comprises a subject who is at risk of developing a neurodegenerative disease or condition. Or the individual may comprise a genetic mutation associated with a neurodegenerative disorder (e.g., genetic mutations in Apolipoprotein E (APOE4), Presenilin 1 and 2, Amyloid precursor protein (APP), or TREM2 (Triggering Receptor Expressed On Myeloid Cells 2). In certain embodiments, the neurodegenerative disease or condition may be Alzheimer's disease. In some embodiments, the disease or condition is Parkinson's disease. In some embodiments, the disease or condition is Huntington's disease. In some embodiments, the disease or condition is amyotrophic lateral sclerosis (ALS). In some embodiments, the disease or condition is another amyloid beta-related neurodegenerative disorder. The present invention is not limited to the aforementioned diseases or conditions.

The present invention may further feature a composition (e.g., a therapeutic composition) comprising a sufficient number of modified cells described herein for use in a method of treating neurodegenerative disorders or conditions. In some embodiments, the present invention features a composition (e.g., a therapeutic composition) comprising a sufficient number of modified cells described herein for use in a method of treating a disease or condition associated with amyloid beta pathology.

In some embodiments, the present invention features the use of modified cells described herein in the manufacturing of a therapeutic composition for the treatment of neurodegenerative disorders or conditions. In other embodiments, the present invention features the use of modified cells described herein in the manufacturing of a therapeutic composition for the treatment of Alzheimer's Disease. In other embodiments, the present invention features the use of modified cells described herein in the manufacturing of a therapeutic composition for the treatment of Parkinson's Disease. In other embodiments, the present invention features the use of modified cells described herein in the manufacturing of a therapeutic composition for the treatment of Huntington's Disease. In other embodiments, the present invention features the use of modified cells described herein in the manufacturing of a therapeutic composition for the treatment of ALS.

As has been described herein, the present invention features a human induced microglia-like (iMGL) cell that expresses and presents a therapeutic molecule comprising membrane-bound neprilysin or expresses and secretes a therapeutic molecule comprising secreted neprilysin.

As has been described herein, the present invention features a human induced microglia-like (iMGL) cell that expresses and presents or expresses and secretes a therapeutic molecule comprising: membrane-bound neprilysin, secreted neprilysin, TREM2, APOE, LRP1, insulin degrading enzyme, endothelin-converting enzyme, plasminogen activator, angiotensin-converting enzyme, or a matrix metalloproteinase.

Referring to the appropriate embodiments herein, in some embodiments, the promoter is configured to activate transcription of the therapeutic molecule upon (i) proximity of the modified cell to 3-amyloid (Aβ) peptide plaques, soluble Aβ monomers, insoluble Aβ monomers, Aβ oligomers, pyroglutamate Aβ, protofibrils, or fibrils comprising Aβ of or a fragment thereof; or (ii) contact of the modified cell with β-amyloid (Aβ) peptide plaques, soluble Aβ monomers, insoluble Aβ monomers, Aβ oligomers, pyroglutamate Aβ, protofibrils, or fibrils comprising Aβ or a fragment thereof. In some embodiments, the cell expresses P2RY12 and TREM2.

Referring to the appropriate embodiments herein, in some embodiments, the cell comprises a nucleic acid sequence encoding the therapeutic molecule; and a promoter selected from a CD9 gene promoter, a LGALS3 gene promoter, an HLA-DRB gene promoter, or a CD11c gene promoter, wherein the promoter is operatively linked to the nucleic acid sequence encoding the therapeutic molecule. In some embodiments, the promoter comprises the CD9 gene promoter. In some embodiments, the promoter comprises the LGALS3 gene promoter. In some embodiments, the promoter comprises the HLA-DRB gene promoter. In some embodiments, the promoter comprises the CD11c gene promoter. In some embodiments, the promoter is one of the aforementioned promoters and the therapeutic molecule comprises membrane-bound neprilysin or secreted neprilysin. In some embodiments, the promoter is one of the aforementioned promoters and the therapeutic molecule comprises one of SEQ ID NO: 1, SEQ ID NO: 2, or SEQ ID NO: 3. In some embodiments, the promoter is one of the aforementioned promoters and the therapeutic molecule comprises TREM2. In some embodiments, the promoter is one of the aforementioned promoters and the therapeutic molecule comprises insulin degrading enzyme.

Referring to the appropriate embodiments herein, in some embodiments, the nucleic acid sequence is inserted into the genome of the cell. In some embodiments, the nucleic acid sequence is inserted downstream of a locus controlled by the promoter such that the nucleic acid sequence is in-frame with a coding sequence in an exon of the locus. In some embodiments, the nucleic acid sequence is inserted within the locus controlled by the promoter such that the nucleic acid sequence is joined, inframe, with at least part of the coding sequence of the locus. In some embodiments, a first polynucleotide encoding a protease cleavage site, a ribosomal skipping sequence, or a self-cleaving peptide is inserted between the coding sequence of the exon and the nucleic acid sequence encoding the therapeutic protein. In some embodiments, the self-cleaving peptide is P2A. In some embodiments, a second polynucleotide encoding a secreted peptide signal sequence is inserted at the 5′ end of the nucleic acid sequence encoding the therapeutic protein.

Referring to the appropriate embodiments herein, in some embodiments, the therapeutic protein affects one or more amyloid-beta (Aβ)-related pathologies. In some embodiments, the Aβ-related pathology comprises β-amyloid (Aβ) peptide plaques, soluble Aβ monomers, insoluble Aβ monomers, Aβ oligomers, pyroglutamate Aβ, protofibrils, or fibrils comprising Aβ of varying lengths. In some embodiments, the therapeutic molecule reduces the amount of Aβ peptide in Aβ peptide plaques in the brain of an individual. In some embodiments, the therapeutic molecule reduces the size or number of soluble Aβ monomers, insoluble Aβ monomers, Aβ oligomers, pyroglutamate Aβ, protofibrils, or fibrils comprising Aβ of varying lengths. In some embodiments, the therapeutic molecule enhances amyloid proteolysis. In some embodiments, the therapeutic molecule enhances microglial phagocytosis of amyloid beta.

As has been described herein, the present invention features a modified cell that expresses and presents or secretes a therapeutic molecule when the cell is proximal to or in contact with amyloid-beta (Aβ)-related pathology, wherein the therapeutic molecule reduces or eliminates an Aβ-related pathology phenotype or ameliorates a symptom of the Aβ-related pathology.

As has been described herein, the present invention features a modified cell that expresses and presents or secretes a therapeutic molecule when the cell is proximal to, or in contact with amyloid-beta (Aβ)-related pathology, wherein the therapeutic molecule reduces or eliminates at least one aspect of the Aβ-related pathology.

As has been described herein, the present invention features a modified cell that expresses and presents or secretes a therapeutic molecule when the cell is proximal to, or in contact with an amyloid-beta (Aβ) aggregate, wherein the therapeutic molecule reduces or eliminates an amyloid beta-related pathology or ameliorates a symptom thereof.

As has been described herein, the present invention features a modified cell that expresses and presents or secretes a therapeutic molecule when the cell is proximal to, or in contact with an amyloid-beta (Aβ) aggregate, plaque, oligomer, or fibril, wherein the therapeutic molecule reduces or eliminates an amyloid beta-related pathology or ameliorates a symptom thereof.

Referring to the appropriate embodiments herein, in some embodiments, the amyloid beta-related pathology phenotype comprises a phenotype selected from the group consisting of memory problems, cognitive problems, vision problems, behavioral changes, personality changes, depression, and seizures.

Referring to the appropriate embodiments herein, in some embodiments, the cell comprises a nucleic acid sequence encoding the therapeutic molecule, wherein the nucleic acid sequence is operatively linked to a promoter responsive to amyloid beta-related pathology.

As has been described herein, the present invention features a modified cell that expresses and presents or secretes a therapeutic molecule, said modified cell comprises: a nucleic acid sequence encoding a therapeutic molecule, the therapeutic molecule cleaves amyloid beta, binds to amyloid beta, enhances amyloid proteolysis, enhances amyloid proteolysis, enhances microglial phagocytosis of amyloid beta, or a combination thereof; and a promoter selected from a CD9 gene promoter, a LGALS3 gene promoter, an HLA-DRB gene promoter, a TREM2 gene promoter, or a CD11c gene promoter, the promoter is operatively linked to the nucleic acid sequence encoding the therapeutic molecule.

As has been described herein, the present invention features a modified cell that expresses and presents or secretes a therapeutic molecule, said modified cell comprises: a nucleic acid sequence encoding a therapeutic molecule, the therapeutic molecule cleaves amyloid beta, binds to amyloid beta, enhances amyloid proteolysis, enhances amyloid proteolysis, enhances microglial phagocytosis of amyloid beta, or a combination thereof; and a promoter selected from a DCSTAMP gene promoter, a CD9 gene promoter, a CD44 gene promoter, an LGALS3 gene promoter, an SPP1 gene promoter, a GPNMB gene promoter, an HLA-DRB gene promoter, an LPL gene promoter, a LIPA gene promoter, a FABP3 gene promoter, an MS4A6A gene promoter, a CXCR4 gene promoter, a CHI3L1 gene promoter, an OLR1 gene promoter promoter, a CD36 gene promoter, a SLAMF8 gene promoter, a TREM2 gene promoter, an MSR1 gene promoter, a B2M gene promoter, an ITGAX gene promoter, and a MITF gene promoter; the promoter is operatively linked to the nucleic acid sequence encoding the therapeutic molecule.

As has been described herein, the present invention features a modified cell that expresses and presents or secretes a therapeutic molecule comprising membrane-bound neprilysin or secreted neprilysin, said modified cell comprising: a nucleic acid sequence encoding the therapeutic molecule; and a promoter operatively linked to the nucleic acid sequence encoding the therapeutic molecule, the promoter is configured to activate transcription of the therapeutic molecule upon (i) proximity of the cell to β-amyloid (Aβ) peptide plaques, soluble Aβ monomers, insoluble Aβ monomers, Aβ oligomers, pyroglutamate Aβ, protofibrils, or fibrils comprising Aβ of or a fragment thereof; or (ii) contact of the cell with β-amyloid (Aβ) peptide plaques, soluble Aβ monomers, insoluble Aβ monomers, Aβ oligomers, pyroglutamate Aβ, protofibrils, or fibrils comprising Aβ or a fragment thereof.

As has been described herein, the present invention features a modified cell that expresses and presents or secretes a therapeutic molecule comprising membrane-bound neprilysin, secreted neprilysin, TREM2, or insulin degrading enzyme, said modified cell comprising: a nucleic acid sequence encoding the therapeutic molecule; and a promoter operatively linked to the nucleic acid sequence encoding the therapeutic molecule, the promoter is configured to activate transcription of the therapeutic molecule upon (i) proximity of the cell to β-amyloid (Aβ) peptide plaques, soluble Aβ monomers, insoluble Aβ monomers, Aβ oligomers, pyroglutamate Aβ, protofibrils, or fibrils comprising Aβ of or a fragment thereof; or (ii) contact of the cell with β-amyloid (Aβ) peptide plaques, soluble Aβ monomers, insoluble Aβ monomers, Aβ oligomers, pyroglutamate Aβ, protofibrils, or fibrils comprising Aβ or a fragment thereof.

As has been described herein, the present invention features a microglia-like (MGL) cell that expresses and presents or secretes a therapeutic molecule that cleaves amyloid beta, binds to amyloid beta, enhances amyloid proteolysis, enhances enhances microglial phagocytosis of amyloid beta, or a combination thereof, said cell comprising: a nucleic acid sequence encoding the therapeutic molecule; and a promoter operatively linked to the nucleic acid sequence encoding the therapeutic molecule, the promoter is configured to activate transcription of the therapeutic molecule upon (i) proximity of the cell to β-amyloid (Aβ) peptide plaques, soluble Aβ monomers, insoluble Aβ monomers, Aβ oligomers, pyroglutamate Aβ, protofibrils, or fibrils comprising Aβ of or a fragment thereof; or (ii) contact of the cell with β-amyloid (Aβ) peptide plaques, soluble Aβ monomers, insoluble Aβ monomers, Aβ oligomers, pyroglutamate Aβ, protofibrils, or fibrils comprising Aβ or a fragment thereof.

Referring to the appropriate embodiments herein, in some embodiments, the promoter is configured to activate transcription of the therapeutic molecule upon (i) proximity of the cell to β-amyloid (Aβ) peptide plaques, soluble Aβ monomers, insoluble Aβ monomers, Aβ oligomers, pyroglutamate Aβ, protofibrils, or fibrils comprising Aβ of or a fragment thereof; or (ii) contact of the cell with β-amyloid (Aβ) peptide plaques, soluble Aβ monomers, insoluble Aβ monomers, Aβ oligomers, pyroglutamate Aβ, protofibrils, or fibrils comprising Aβ or a fragment thereof. In some embodiments, the cell expresses P2RY12 and TREM2.

Referring to the appropriate embodiments herein, in some embodiments, the cell is a migratory cell or is produced from a lineage of cells that can differentiate into migratory cells. In some embodiments, the cell is a pluripotent stem cell (PSC), an induced pluripotent stem cells (iPSC), a myeloid progenitor cell, an erythromyeloid progenitor, a hematopoietic stem cell, a hematopoietic progenitor cell, a lymphoid progenitor cell, a megakaryocyte-erythroid (mk-ery), a cord-blood stem cell, or an embryonic stem cell. In some embodiments, the cell is a monocyte. In some embodiments, the cell is a bone marrow-derived hematopoietic precursor cell. In some embodiments, the cell is a neural stem cell. In some embodiments, the cell is an iPSC-derived microglial cell. In some embodiments, the cell is an iPSC-derived hematopoietic precursor cell. In some embodiments, the cell is a hematopoietic precursor cell. In some embodiments, the cell is a microglia-like (MGL) cell. In some embodiments, the cell is a human induced microglia-like cell (hiMGL). In some embodiments, the MGL cell is a human induced pluripotent stem-cell-derived MGL cell. In some embodiments, the cell is a microglia-like (MGL) cell capable of phagocytosing human synaptosomes. In some embodiments, the cell is a microglia-like (MGL) cell capable of phagocytosing amyloid Aβ fibers. In some embodiments, the cell is a microglia-like (MGL) cell capable of migrating to an injury site.

Referring to the appropriate embodiments herein, in some embodiments, the therapeutic protein is a membrane bound protein. In some embodiments, the therapeutic protein is a secreted protein. In some embodiments, the therapeutic protein is a metalloprotease. In some embodiments, the therapeutic protein comprises neprilysin activity. In some embodiments, the therapeutic molecule comprises membrane-bound neprilysin or secreted neprilysin. In some embodiments, the therapeutic molecule comprises a peptide according to SEQ ID NO: 1, SEQ ID NO: 2, or SEQ ID NO: 3. In some embodiments, the therapeutic molecule comprises TREM2, APOE, LRP1, or insulin degrading enzyme, endothelin-converting enzyme, plasminogen activator, angiotensin-converting enzyme, or a matrix metalloproteinase.

Referring to the appropriate embodiments herein, in some embodiments, the promoter is selected from a CD9 gene promoter, a LGALS3 gene promoter, a TREM2 gene promoter, an HLA-DRB gene promoter, or a CD11c gene promoter. In some embodiments, the promoter is selected from: a DCSTAMP gene promoter, a CD9 gene promoter, a CD44 gene promoter, an LGALS3 gene promoter, an SPP1 gene promoter, a GPNMB gene promoter, an HLA-DRB gene promoter, an LPL gene promoter, a LIPA gene promoter, a FABP3 gene promoter, an MS4A6A gene promoter, a CXCR4 gene promoter, a CHI3L1 gene promoter, an OLR1 gene promoter promoter, a CD36 gene promoter, a SLAMF8 gene promoter, a TREM2 gene promoter, an MSR1 gene promoter, a B2M gene promoter, an ITGAX gene promoter, and a MITF gene promoter. In some embodiments, the nucleic acid sequence is inserted into the genome of the cell. In some embodiments, the nucleic acid sequence is inserted downstream of a locus controlled by the promoter such that the nucleic acid sequence is in-frame with a coding sequence in an exon of the locus. In some embodiments, the nucleic acid sequence is inserted within the locus controlled by the promoter such that the nucleic acid sequence is joined, inframe, with at least part of the coding sequence of the locus. In some embodiments, a first polynucleotide encoding a protease cleavage site, a ribosomal skipping sequence, or a self-cleaving peptide is inserted between the coding sequence of the exon and the nucleic acid sequence encoding the therapeutic protein. In some embodiments, the self-cleaving peptide is P2A. In some embodiments, a second polynucleotide encoding a secreted peptide signal sequence is inserted at the 5′ end of the nucleic acid sequence encoding the therapeutic protein. In some embodiments, the therapeutic protein affects one or more amyloid-beta (Aβ)-related pathologies. In some embodiments, the Aβ-related pathology comprises β-amyloid (Aβ) peptide plaques, soluble Aβ monomers, insoluble Aβ monomers, Aβ oligomers, pyroglutamate Aβ, protofibrils, or fibrils comprising Aβ of varying lengths. In some embodiments, the therapeutic molecule reduces the amount of Aβ peptide in Aβ peptide plaques in the brain of an individual. In some embodiments, the therapeutic molecule reduces the size or number of soluble Aβ monomers, insoluble Aβ monomers, Aβ oligomers, pyroglutamate Aβ, protofibrils, or fibrils comprising Aβ of varying lengths; enhances amyloid proteolysis; enhances microglial phagocytosis of amyloid beta, or a combination thereof.

As has been described herein, the present invention features a composition comprising the cell of any of the embodiments disclosed herein.

As has been described herein, the present invention features a kit comprising the cell of any of the embodiments disclosed herein or the composition of any of the embodiments disclosed herein.

As has been described herein, the present invention features a composition for use in a method of treating a disease or condition associated with amyloid beta-related pathology or ameliorating symptoms or pathological processes associated with a disease or condition associated with amyloid beta-related pathology, said composition comprising a cell any of the embodiments disclosed herein.

As has been described herein, the present invention features a method of treating an individual having a disease or condition associated with amyloid beta-related pathology, said method comprising administering to at least one target brain region in a brain of the individual the cell of any of the embodiments disclosed herein or the composition of any of the embodiments disclosed herein.

As has been described herein, the present invention features a method of reducing: an amount of Aβ peptide in Aβ peptide plaques; and/or a size or number of soluble Aβ monomers, insoluble Aβ monomers, Aβ oligomers, pyroglutamate Aβ, protofibrils, or fibrils comprising Aβ of varying lengths; in a brain of an individual; comprising administering to at least one target brain region in a brain of the individual the cell any of the embodiments disclosed herein, or the composition of any of the embodiments disclosed herein, to the individual.

As has been described herein, the present invention features a method of reducing neuronal or synaptic loss in a subject in need thereof, the subject having an amyloid beta-related pathology comprising Aβ plaques in brain tissue, said method comprising administering to at least one target brain region in a brain of the individual the cell of any of the embodiments disclosed herein, or the composition of any of the embodiments disclosed herein, to the individual.

As has been described herein, the present invention features a method of preventing or attenuating onset of a neurodegenerative disorder or condition associated with amyloid beta pathology, said method comprising administering to at least one target brain region in a brain of the individual the cell any of the embodiments disclosed herein or the composition of any of the embodiments disclosed herein.

Referring to the appropriate embodiments herein, in some embodiments, the at least one target brain region comprises a cerebral cortex or subregion thereof, a hippocampus or subregion thereof, a cerebral ventricle, a basal ganglia or basal ganglia nucleus thereof, an entorhinal cortex, a medial prefrontal cortex or subregion thereof, an anterior cingulate cortex or subregion thereof, a primary sensory cortex or sensory association cortex, a fornix, a septum, or a combination thereof. In some embodiments, the hippocampus or subregion thereof comprises a CA1 region of the hippocampus, a CA3 region of the hippocampus, a dentate gyrus of the hippocampus, a CA2 region of the hippocampus, a septal region or fornix of the hippocampus. In some embodiments, the cerebral ventricle comprises a lateral ventricle, a third ventricle, a fourth ventricle, or a combination thereof. In some embodiments, the basal ganglia nucleus comprises a globus pallidus, a substantia nigra pars compacta, a substantia nigra pars reticulata, a striatum, a caudate putamen, a subthalamic nucleus.

Referring to the appropriate embodiments herein, in some embodiments, the amyloid beta-related pathology is Alzheimer's disease. In some embodiments, the amyloid beta-related pathology is Parkinson's disease. In some embodiments, the amyloid beta-related pathology is Huntington's disease. In some embodiments, the amyloid beta-related pathology is amyotrophic lateral sclerosis (ALS). In some embodiments, the method inhibits expansion or progression of the amyloid beta-related pathology. In some embodiments, the method inhibits the expansion of Abeta fibers. In some embodiments, the method reduces synaptic loss.

Referring to the appropriate embodiments herein, in some embodiments, the cell is administered prior to onset of Aβ-related pathology. In some embodiments, the cell is administered prior to a presence of β-amyloid (Aβ) peptide plaques, soluble Aβ monomers, insoluble Aβ monomers, Aβ oligomers, pyroglutamate Aβ, protofibrils, or fibrils comprising Aβ or a fragment thereof in the individual. In some embodiments, the cell is administered after detection of a presence of β-amyloid (Aβ) peptide plaques, soluble Aβ monomers, insoluble Aβ monomers, Aβ oligomers, pyroglutamate Aβ, protofibrils, or fibrils comprising Aβ or a fragment thereof in the individual. In some embodiments, the cell is administered via stereotactic injection into the brain of the individual. In some embodiments, the cell is administered to the individual prior to adulthood. In some embodiments, the individual is at-risk of developing a neurodegenerative disease or condition. In some embodiments, the individual comprises a genetic mutation associated with a neurodegenerative disorder.

Referring to the appropriate embodiments herein, in some embodiments, the amount of Aβ peptide or Aβ peptide plaques is determined using amyloid beta PET imaging, a histological method, an immunoblotting method, an amyloid beta staining method, or a combination thereof. In some embodiments, the immunoblotting method detects a synaptic marker. In some embodiments, the staining method detects amyloid beta. In some embodiments, the staining method comprises Golgi staining for measuring synapse number. In some embodiments, the amount of Aβ peptide in Aβ peptide plaques is determined using magnetic resonance imaging (MRI).

As has been described herein, the present invention features the use of the cell of of any of the embodiments disclosed herein, the composition of any of the embodiments disclosed herein, or the kit of any of the embodiments disclosed herein, in: reducing the amount of Aβ peptide in Aβ peptide plaques; and/or reducing the size or number of soluble Aβ monomers, insoluble Aβ monomers, Aβ oligomers, pyroglutamate Aβ, protofibrils, or fibrils comprising Aβ of varying lengths Aβ peptide deposition. In some embodiments, the use is for a method comprising transplanting hiMGLs into a brain tissue of an individual.

As has been described herein, the present invention features the use of the cell of any of the embodiments disclosed herein, the composition of any of the embodiments disclosed herein, or the kit of any of the embodiments disclosed herein, in treating a disease associated with amyloid beta-related pathology, or ameliorating symptoms or pathological processes associated with amyloid beta-related pathology.

As has been described herein, the present invention features a human induced microglia-like (iMGL) cell that expresses and presents or expresses and secretes a therapeutic molecule comprising membrane-bound neprilysin, secreted neprilysin, TREM2, APOE, LRP1, insulin degrading enzyme, endothelin-converting enzyme, plasminogen activator, angiotensin-converting enzyme, or a matrix metalloproteinase. In some embodiments, the cell expresses and presents or expresses and secretes the therapeutic molecule when contacted by β-amyloid (A) peptide plaques, soluble Aβ monomers, insoluble Aβ monomers, Aβ oligomers, pyroglutamate Aβ, protofibrils, or fibrils comprising Aβ of varying lengths. In some embodiments, the cell comprises: a nucleic acid sequence encoding the therapeutic molecule; and a promoter selected from a CD9 gene promoter, a LGALS3 gene promoter, an HLA-DRB gene promoter, or a CD11c gene promoter, wherein the promoter is operatively linked to the nucleic acid sequence encoding the therapeutic molecule.

Referring to the appropriate embodiments herein, in some embodiments, the therapeutic molecule binds to Aβ peptide. Referring to the appropriate embodiments herein, in some embodiments, the therapeutic molecule cleaves Aβ peptide.

In some embodiments, Aβ peptide refers to Aβ-40. the cell of claim 4, wherein Aβ peptide refers to Aβ-42.

Referring to the appropriate embodiments herein, in some embodiments, the therapeutic molecule cleaves amyloid beta, binds to amyloid beta, enhances amyloid proteolysis, enhances amyloid proteolysis, enhances microglial phagocytosis of amyloid beta, or a combination thereof.

As has been described herein, the present invention features cells, compositions, and methods, wherein the nucleic acid sequence is inserted into the genome of the cell. For any of the appropriate embodiments herein, in some embodiments, the nucleic acid sequence is inserted downstream of a locus controlled by the promoter such that the nucleic acid sequence is in-frame with a coding sequence in an exon of the locus. In some embodiments, the nucleic acid sequence is inserted within the locus controlled by the promoter such that the nucleic acid sequence is joined, inframe, with at least part of the coding sequence of the locus. In some embodiments, the exon is the first exon in the locus. In some embodiments, the exon is the last exon in the locus.

As has been described herein, the present invention features the use of the cells of any of the embodiments or claims herein, the compositions of any of the embodiments or claims herein, or the kits of any of the embodiments or claims herein, in a method of treating a disease or condition associated with amyloid beta-related pathology or ameliorating symptoms or pathological processes associated with a disease or condition associated with amyloid beta-related pathology.

As has been described herein, the present invention features a method of treating a neurodegenerative disease or condition, said method comprising: administering to the individual a cell of any of the embodiments or claims herein or a composition of any of the embodiments or claims herein.

Referring to any of the appropriate embodiments herein, in some embodiments, neurodegenerative disease or condition is associated with amyloid beta-related pathology. In some embodiments, amyloid beta-related pathology is Alzheimer's disease. In some embodiments, amyloid beta-related pathology is Parkinson's disease. In some embodiments, amyloid beta-related pathology is Huntington's disease. In some embodiments, amyloid beta-related pathology is amyotrophic lateral sclerosis (ALS).

Referring to any of the appropriate embodiments herein, the at least one target brain region comprises a cerebral cortex or subregion thereof. In some embodiments, the at least one target brain region comprises a hippocampus or subregion thereof. In some embodiments, the at least one target brain region comprises a CA1 region of the hippocampus. In some embodiments, the at least one target brain region comprises a CA3 region of the hippocampus. In some embodiments, the at least one target brain region comprises a dentate gyrus of the hippocampus. In some embodiments, the at least one target brain region comprises a CA2 region of the hippocampus. In some embodiments, the at least one target brain region comprises a septal region or fornix of the hippocampus. In some embodiments, the at least one target brain region comprises a cerebral ventricle. In some embodiments, the cerebral ventricle comprises a lateral ventricle, a third ventricle, a fourth ventricle, or a combination thereof. In some embodiments, the at least one target brain region comprises a basal ganglia or basal ganglia nucleus thereof. In some embodiments, the basal ganglia nucleus comprises a globus pallidus. In some embodiments, the basal ganglia nucleus comprises a substantia nigra pars compacta or substantia nigra pars reticulata. In some embodiments, the basal ganglia nucleus comprises a striatum. In some embodiments, the basal ganglia nucleus comprises a caudate putamen. In some embodiments, the basal ganglia nucleus comprises a subthalamic nucleus. In some embodiments, the at least one target brain region comprises an entorhinal cortex. In some embodiments, the at least one target brain region comprises a medial prefrontal cortex or subregion thereof. In some embodiments, the at least one target brain region comprises an anterior cingulate cortex or subregion thereof. In some embodiments, the at least one target brain region comprises a primary sensory cortex or sensory association cortex. In some embodiments, the at least one target brain region comprises a cerebral cortex or subregion thereof, a hippocampus or subregion thereof, a cerebral ventricle, or a combination thereof. In some embodiments, the at least one target brain region comprises an anterior cingulate cortex, an entorhinal cortex, a dentate gyrus, a CA1 region of the hippocampus, a CA3 region of the hippocampus, a fomix, a primary sensory cortex, a sensory association cortex, a septum, or a CA2 region of the hippocampus.

The present invention also features a method of producing a therapeutic modified cell, comprising introducing into a cell a nucleic acid sequence encoding a therapeutic molecule, wherein the nucleic acid sequence is operatively linked to a promoter that is responsive to amyloid beta-related pathology, and wherein the therapeutic protein alters an amyloid beta-related pathology phenotype or at least one aspect of the amyloid beta-related pathology.

The present invention also features a method of producing a cell population comprising human microglial-like cells (iMGLs), the method comprising: contacting human induced hematopoietic progenitor cells (iHPCs) with a microglial differentiating medium comprising CSF-1, IL-34, and TGFβ1 or CSF-1, IL-34, and a TGFβ mimetic to differentiate the iHPCs into iMGLs, and introducing a nucleic acid encoding a protease that cleave amyloid Aβ fibers into the iMGLs.

The present invention also features a method of producing a cell population comprising human microglial-like cells (iMGLs), the method comprising: contacting human induced hematopoietic progenitor cells (iHPCs) with a microglial differentiating medium comprising CSF-1, IL-34, and TGFβ1 or CSF-1, IL-34, and a TGFβ mimetic to differentiate the iHPCs into iMGLs, and introducing a nucleic acid encoding a therapeutic molecule that affects at least one aspect of an amyloid beta-related pathology into the iMGLs.

The present invention also features a method of producing a therapeutic cell population comprising human microglial-like cells (iMGLs), the method comprising: plating human induced hematopoietic progenitor cells (iHPCs) on a basement membrane protein-coated culture dish; and contacting the human iHPCs with a microglial differentiating medium comprising CSF-1 or IL-34 to differentiate the iHPCs into iMGLs; and introducing a nucleic acid encoding a therapeutic molecule that affects at least one aspect of an amyloid beta-related pathology into the iMGLs.

The present invention also features a method of producing a therapeutic cell population comprising human microglial-like cells (iMGLs), the method comprising: plating human induced hematopoietic progenitor cells (iHPCs) on a basement membrane protein-coated culture dish; contacting the human iHPCs with a microglial differentiating medium comprising CSF-1 or IL-34 to differentiate the iHPCs into iMGLs; and introducing a nucleic acid encoding a protease that cleaves amyloid Aβ fibers into the iMGLs.

For any of the appropriate embodiments, in some embodiments, the therapeutic molecule binds to Aβ peptide, cleaves Aβ peptide, reduces the amount of Aβ peptide in Aβ peptide plaques, reduces the size or number of soluble Aβ monomers, insoluble Aβ monomers, Aβ oligomers, pyroglutamate Aβ, protofibrils, or fibrils comprising Aβ of varying lengths, enhances amyloid proteolysis, enhances microglial phagocytosis of amyloid beta, or a combination thereof.

For any of the appropriate embodiments, in some embodiments, the cell comprises a nucleic acid sequence encoding the therapeutic molecule, wherein the nucleic acid sequence is operatively linked to a promoter responsive to amyloid beta-related pathology.

For any of the appropriate embodiments, in some embodiments, the promoter is an endogenous promoter. For any of the appropriate embodiments, in some embodiments, the promoter is a non-endogenous promoter.

As has been described herein, the present invention features a method of treating an individual having a disease or condition associated with amyloid beta-related pathology, said method comprising administering to at least one target brain region in a brain of the individual a cell of any of the embodiments or claims herein, or a compositions of any of the embodiments or claims herein. The present invention also features a method of reducing: an amount of Aβ peptide in Aβ peptide plaques; and/or a size or number of soluble Aβ monomers, insoluble Aβ monomers, Aβ oligomers, pyroglutamate Aβ, protofibrils, or fibrils comprising Aβ of varying lengths; in a brain of an individual; comprising administering to at least one target brain region in a brain of the individual a cell of any of the embodiments or claims herein, or a compositions of any of the embodiments or claims herein to the individual. The present invention also features a method of reducing neuronal or synaptic loss in a subject in need thereof, the subject having an amyloid beta-related pathology comprising Aβ plaques in brain tissue, said method comprising administering to at least one target brain region in a brain of the individual a cell of any of the embodiments or claims herein, or a compositions of any of the embodiments or claims herein, to the individual. The present invention also features a method of preventing or attenuating onset of a neurodegenerative disorder or condition associated with amyloid beta pathology, said method comprising administering to at least one target brain region in a brain of the individual a cell of any of the embodiments or claims herein, or a compositions of any of the embodiments or claims herein. In some embodiments, the amyloid beta-related pathology is Alzheimer's disease. In some embodiments, the amyloid beta-related pathology is Parkinson's disease. In some embodiments, the amyloid beta-related pathology is Huntington's disease. In some embodiments, the amyloid beta-related pathology is amyotrophic lateral sclerosis (ALS). In some embodiments, the amyloid beta-related pathology is Alzheimer's disease, Parkinson's disease, Hungtington's disease, or ALS. In some embodiments, the amyloid beta related pathology is a different neurological disease. In some embodiments, the methods inhibit expansion or progression of the amyloid beta-related pathology. In some embodiments, the method inhibits the expansion of amyloid beta fibers. In some embodiments, the method reduces synaptic loss. In some embodiments, the individual is a subject who is at-risk of developing a neurodegenerative disease or condition. In some embodiments, the individual comprises a genetic mutation associated with a neurodegenerative disorder. In some embodiments, the cell is administered prior to adulthood.

Detection or monitoring of levels of amyloid beta peptide and/or plaques may be performed using imaging (e.g., amyloid beta PET imaging, MRI), a histological method, an immunoblotting method, an amyloid beta staining method, or a combination thereof. In some embodiments, the immunoblotting method detects a synaptic marker. In some embodiments, the staining method detects amyloid beta. In some embodiments, the staining method comprises Golgi staining for measuring synapse number. In some embodiments, the amount of Aβ peptide in Aβ peptide plaques is determined using magnetic resonance imaging (MRI).

As has been described herein, the present invention features a method of treating an individual having a disease or condition associated with amyloid beta-related pathology, said method comprising administering to at least one target brain region in a brain of the individual a cell of any of the embodiments or claims herein, or a compositions of any of the embodiments or claims herein. The present invention also features a method of reducing: an amount of Aβ peptide in Aβ peptide plaques; and/or a size or number of soluble Aβ monomers, insoluble Aβ monomers, Aβ oligomers, pyroglutamate Aβ, protofibrils, or fibrils comprising Aβ of varying lengths; in a brain of an individual; comprising administering to at least one target brain region in a brain of the individual a cell of any of the embodiments or claims herein, or a compositions of any of the embodiments or claims herein to the individual. The present invention also features a method of reducing neuronal or synaptic loss in a subject in need thereof, the subject having an amyloid beta-related pathology comprising Aβ plaques in brain tissue, said method comprising administering to at least one target brain region in a brain of the individual a cell of any of the embodiments or claims herein, or a compositions of any of the embodiments or claims herein, to the individual. The present invention also features a method of preventing or attenuating onset of a neurodegenerative disorder or condition associated with amyloid beta pathology, said method comprising administering to at least one target brain region in a brain of the individual a cell of any of the embodiments or claims herein, or a compositions of any of the embodiments or claims herein. In some embodiments, the at least one target brain region comprises a cerebral cortex or subregion thereof, a hippocampus or subregion thereof, a cerebral ventricle, a basal ganglia or basal ganglia nucleus thereof, an entorhinal cortex, a medial prefrontal cortex or subregion thereof, an anterior cingulate cortex or subregion thereof, a primary sensory cortex or sensory association cortex, a fornix, a septum, or a combination thereof. In some embodiments, the hippocampus or subregion thereof comprises a CA1 region of the hippocampus, a CA3 region of the hippocampus, a dentate gyrus of the hippocampus, a CA2 region of the hippocampus, a septal region or fornix of the hippocampus. In some embodiments, the cerebral ventricle comprises a lateral ventricle, a third ventricle, a fourth ventricle, or a combination thereof.

In some embodiments, the basal ganglia nucleus comprises a globus pallidus, a substantia nigra pars compacta, a substantia nigra pars reticulata, a striatum, a caudate putamen, a subthalamic nucleus.

This written description uses examples to disclose the invention, including the best mode, and to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### EXAMPLES

The following are non-limiting examples of the present invention. It is to be understood that said examples are not intended to limit the present invention in any way. Equivalents or substitutes are within the scope of the present invention.

### Example 1. Mouse Model Description

Microglia are the primary innate immune cells of the brain and play a critical role in maintaining neuronal homeostasis and surveying their local environment for pathogenic agents and neuronal damage. Recently, a chimeric mouse model was able to be developed that allows examination of the interactions between human iPSC-derived microglia and neuropathology (Hasselmann et. al., Neuron, 2019). This model harbors deletions in Rag2 and il2rγ genes and humanized CSF-1 alleles, which has thus shown to be necessary and sufficient to enable long-term engraftment and survival of xenotransplanted human microglia within the murine brain. This model was further crossed with 5×FAD mice (Oakley et. al., J Neurosci, 2006) which overexpress co-integrated transgenes for Familial Alzheimer's Disease (FAD) mutant APP (Swedish, Florida, and London) and mutant FAD PS1 (M146L and L286V) and develops extensive amyloid plaque pathology. To generate chimeric mice, mouse pups are transplanted within 5 days of birth with human pluripotent stem cell derived microglia leading to robust 60-80% human microglia chimerism within the forebrain (Hasselmann et. al., Neuron, 2019). In this model, after xenotransplantation, human iPSC-derived microglia (xMGs) disperse widely, mature into homeostatic microglia that exhibit in vivo-like transcriptional profiles and thrive.

### Example 2. Transplanted iMGLs Migrate to and Respond to Aβ Pathology

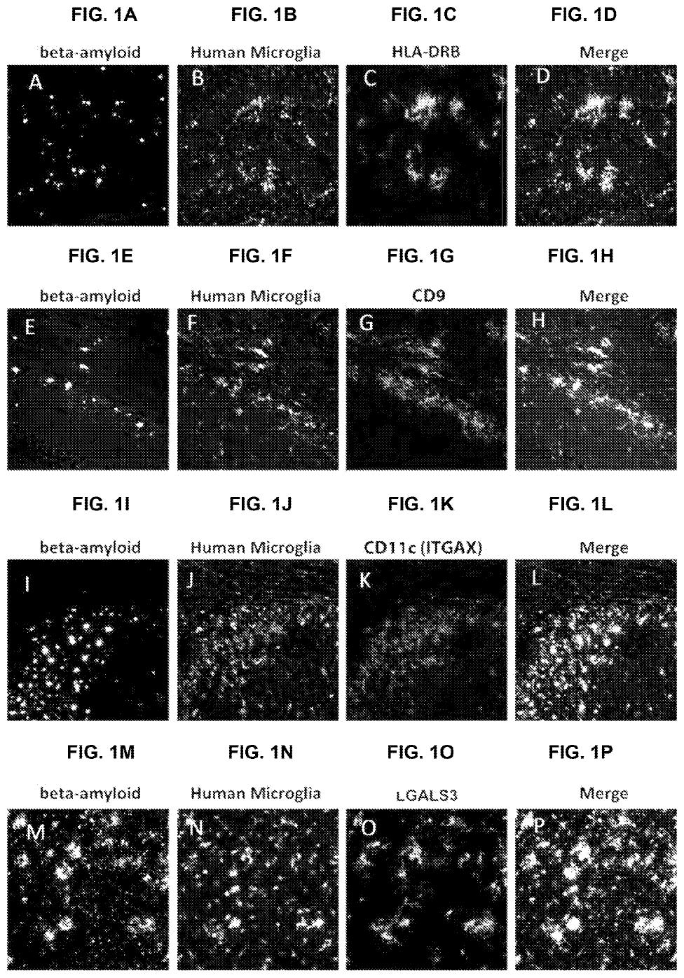

Understanding how iPSC-derived microglia respond to amyloid beta-related pathology, e.g., Alzheimer's disease-associated (AD) pathology, would enable the rational design of gene expression modules to regulate the expression of therapeutic agents. To this end, 5×fAD transgenic mice were crossed onto a MITRG (M-CSF^{h}, IL-3/GM-CSF^{h}, and TPO^{h}Rag2^{tm1.1Flv}Il2γ^{tm1.1Flv}) background to establish a xenotransplantation-compatible model that develops substantial Aβ pathology and both synaptic and neuronal loss referred to as 5×-MITRG (5×FAD×M-CSF^{h}, IL-3/GM-CSF^{h} and TPO^{h}Rag2^{tm1.1Flv}Il2γ^{tm1.1Flv}). Using this model, it was demonstrated that iPSC-derived microglia migrate toward Aβ pathology and express unique gene signatures when they encounter Aβ plaques (Hasselmann et al., Neuron, 2019). Human xenotransplanted microglia (xMGs) were isolated from the brains of 9-10 month old 5×-MITRG mice using a negative magnetic sorting approach to deplete all murine cells. Single-cell RNA sequencing of isolated xMGs was then performed using the 10× genomics Chromium platform and used to distinguish the Disease Associated Microglia (DAM) population associated with Aβ pathology in 5×-MITRG mice and to identify mRNAs that are enriched within human DAM microglia. Immunohistochemistry and confocal microscopy was then used to validate and examine the localization of a subset of these DAM genes at the protein level. It was observed that CD9, a tetraspanin protein, was highly and specifically expressed only in microglia that were directly in contact with Aβ plaques (FIGS. 1A-1P and FIGS. 2A-2F), consistent with mouse models and human microglia from Aβ patient brains. These show that iPSC-derived microglia cells can be transplanted into mouse models of Aβ and that Aβ pathology-associated iPSC-derived microglia exquisitely express CD9.

### Example 3. Construction of Neprilysin (NEP) Expressing iPSC-Derived Microglia Under the Control of the CD9 Promoter

This example illustrates the construction of iPSC-derived microglia cells expressing NEP under the control of the endogenous promoter for CD9. Constructs for both a membrane-anchored neprilysin and a secreted form of NEP were produced. Schematics illustrating the design of the CD9 locus containing the coding sequences for a membrane-anchored form of NEP and the secreted form of NEP are shown in FIGS. 3A & 3B, respectively.

iPSCs were collected following Accutase enzymatic digestion for 3 min at 37 C. 250,000 cells were resuspended in a 100 μL nucleofection buffer from Human Stem Cell Nucleofector™ Kit 2 (Lonza). CD9:NEP or CD9:sNEP plasmid Template (2 μg; sequences provided in Table 2) and RNP complex formed by incubating Alt-R® S.p. HiFi Cas9 Nuclease V3 (50 ug; IDTDNA) fused with crRNA:tracrRNA (IDTDNA) duplex (gRNA: 5′ GCTGACTCTAGACCATCTCG CGG (SEQ ID NO: 9)) was combined with the cellular suspension and nucleofected using the Amaxa Nucleofector program B-46. Cells were plated on matrigel-coated plates in TeSR™-E8™ media with 0.25 NM Thiazovivin (STEMCELL Technologies) and CloneR™ (STEMCELL Technologies) overnight to recover. The following day, cells were mechanically replated to 96-well matrigel-coated plates in TeSR™-E8™ media with 0.25 μM Thiazovivin and CloneR supplement for clonal isolation and expansion. Culture media was replenished everyday with fresh medium. Plates were visually screened for single-clone wells after 5 days. Visually clonal wells were passaged with ReLeSR after 10 days. A cell pellet was collected from each well for which genomic DNA was extracted using Extracta DNA prep for PCR (Quantabio) amplification using Taq PCR Master Mix (Thermo Fisher Scientific) to confirm diallelic integration of the CD9:NEP or CD9:sNEP cassette.

These two constructs were then used for differentiation into microglia using fully defined and scalable production of cmicroglia from human pluripotent cell lines (WO2018160496Ac).

Table 2: Shows sequences for that were used to generate CD9:NEP and CD9:sNEP iPSC lines. The NEP and sNEP templates included the following left and right homology arms and the NEP and sNEP DNA sequences were placed between the left and right homolog arms.

| | | SEQ ID |
|---|---|---|
| | Sequence | NO: |
| NEP sequence | gctactaacttcagcctgctgaagcaggctggagacgtggaggagaaccctggacct | 4 |
| | atgggcaagtcagaaagtcagatggatataactgatatcaacactccaaagccaaag | |
| | aagaaacagcgatggactccactggagatcagcctctcggtccttgtcctgctcctcacc | |
| | atcatagctgtgacaatgatcgcactctatgcaacctacgatgatggtatttgcaagtcat | |
| | cagactgcataaaatcagctgctcgactgatccaaaacatggatgccaccactgagcc | |
| | ttgtacagactttttcaaatatgcttgcggaggctggttgaaacgtaatgtcattcccgaga | |
| | ccagctcccgttacggcaactttgacattttaagagatgaactagaagtcgttttgaaaga | |
| | tgtccttcaagaacccaaaactgaagatatagtagcagtgcagaaagcaaaagcattg | |
| | tacaggtcttgtataaatgaatctgctattgatagcagaggtggagaacctctactcaaac | |
| | tgttaccagacatatatgggtggccagtagcaacagaaaactgggagcaaaaatatg | |
| | gtgcttcttggacagctgaaaaagctattgcacaactgaattctaaatatgggaaaaaa | |
| | gtccttattaatttgtttgttggcactgatgataagaattctgtgaatcatgtaattcatattgac | |
| | caacctcgacttggcctcccttctagagattactatgaatgcactggaatctataaagag | |
| | gcttgtacagcatatgtggattttatgatttctgtggccagattgattcgtcaggaagaaag | |
| | attgcccatcgatgaaaaccagcttgctttggaaatgaataaagttatggaattggaaaa | |
| | agaaattgccaatgctacggctaaacctgaagatcgaaatgatccaatgcttctgtataa | |
| | caagatgacattggcccagatccaaaataacttttcactagagatcaatgggaagccat | |
| | tcagctggttgaatttcacaaatgaaatcatgtcaactgtgaatattagtattacaaatgag | |
| | gaagatgtggttgtttatgctccagaatatttaaccaaacttaagcccattcttaccaaatat | |
| | tctgccagagatcttcaaaatttaatgtcctggagattcataatggatcttgtaagcagcct | |
| | cagccgaacctacaaggagtccagaaatgctttccgcaaggccctttatggtacaacct | |
| | cagaaacagcaacttggagacgttgtgcaaactatgtcaatgggaatatggaaaatgc | |
| | tgtggggaggctttatgtggaagcagcatttgctggagagagtaaacatgtggtcgagg | |
| | atttgattgcacagatccgagaagtttttattcagactttagatgacctcacttggatggatg | |
| | ccgagacaaaaaagagagctgaagaaaaggccttagcaattaaagaaaggatcgg | |
| | ctatcctgatgacattgtttcaaatgataacaaactgaataatgagtacctcgagttgaact | |
| | acaaagaagatgaatacttcgagaacataattcaaaatttgaaattcagccaaagtaa | |
| | acaactgaagaagctccgagaaaaggtggacaaagatgagtggataagtggagca | |
| | gctgtagtcaatgcattttactcttcaggaagaaatcagatagtcttcccagccggcattct | |
| | gcagccccccttctttagtgcccagcagtccaactcattgaactatgggggcatcggcat | |
| | ggtcataggacacgaaatcacccatggcttcgatgacaatggcagaaactttaacaaa | |
| | gatggagacctcgttgactggtggactcaacagtctgcaagtaactttaaggagcaatc | |
| | ccagtgcatggtgtatcagtatggaaacttttcctgggacctggcaggtggacagcacct | |
| | taatggaattaatacactgggagaaaacattgctgataatggaggtcttggtcaagcata | |
| | cagagcctatcagaattatattaaaaagaatggcgaagaaaaattacttcctggacttg | |
| | acctaaatcacaaacaactatttttcttgaactttgcacaggtgtggtgtggaacctatagg | |
| | ccagagtatgcggttaactccattaaaacagatgtgcacagtccaggcaatttcaggatt | |
| | attgggactttgcagaactctgcagagttttcagaagcctttcactgccgcaagaattcat | |
| | acatgaatccagaaaagaagtgccgggtttgg | |
| sNEP sequence | gctactaacttcagcctgctgaagcaggctggagacgtggaggagaaccctggacct | 5 |
| | atgggcgccgctagcggccggcgcggccccgggctgctcctgcccctgccactgctgc | |
| | tgctcctgccccctcagcccgccctggcctacgatgatggtatttgcaagtcatcagactg | |
| | cataaaatcagctgctcgactgatccaaaacatggatgccaccactgagccttgtacag | |
| | actttttcaaatatgcttgcggaggctggttgaaacgtaatgtcattcccgagaccagctc | |
| | ccgttacggcaactttgacattttaagagatgaactagaagtcgttttgaaagatgtccttc | |
| | aagaacccaaaactgaagatatagtagcagtgcagaaagcaaaagcattgtacagg | |
| | tcttgtataaatgaatctgctattgatagcagaggtggagaacctctactcaaactgttacc | |
| | agacatatatgggtggccagtagcaacagaaaactgggagcaaaaatatggtgcttct | |
| | tggacagctgaaaaagctattgcacaactgaattctaaatatgggaaaaaagtccttatt | |
| | aatttgtttgttggcactgatgataagaattctgtgaatcatgtaattcatattgaccaacctc | |
| | gacttggcctcccttctagagattactatgaatgcactggaatctataaagaggcttgtac | |
| | agcatatgtggattttatgatttctgtggccagattgattcgtcaggaagaaagattgccca | |
| | tcgatgaaaaccagcttgctttggaaatgaataaagttatggaattggaaaaagaaatt | |
| | gccaatgctacggctaaacctgaagatcgaaatgatccaatgcttctgtataacaagat | |
| | gacattggcccagatccaaaataacttttcactagagatcaatgggaagccattcagct | |
| | ggttgaatttcacaaatgaaatcatgtcaactgtgaatattagtattacaaatgaggaaga | |
| | tgtggttgtttatgctccagaatatttaaccaaacttaagcccattcttaccaaatattctgcc | |
| | agagatcttcaaaatttaatgtcctggagattcataatggatcttgtaagcagcctcagcc | |
| | gaacctacaaggagtccagaaatgctttccgcaaggccctttatggtacaacctcagaa | |
| | acagcaacttggagacgttgtgcaaactatgtcaatgggaatatggaaaatgctgtggg | |
| | gaggctttatgtggaagcagcatttgctggagagagtaaacatgtggtcgaggatttgatt | |
| | gcacagatccgagaagtttttattcagactttagatgacctcacttggatggatgccgaga | |
| | caaaaaagagagctgaagaaaaggccttagcaattaaagaaaggatcggctatcct | |
| | gatgacattgtttcaaatgataacaaactgaataatgagtacctcgagttgaactacaaa | |
| | gaagatgaatacttcgagaacataattcaaaatttgaaattcagccaaagtaaacaact | |
| | gaagaagctccgagaaaaggtggacaaagatgagtggataagtggagcagctgtag | |
| | tcaatgcattttactcttcaggaagaaatcagatagtcttcccagccggcattctgcagcc | |
| | ccccttctttagtgcccagcagtccaactcattgaactatgggggcatcggcatggtcata | |
| | ggacacgaaatcacccatggcttcgatgacaatggcagaaactttaacaaagatgga | |
| | gacctcgttgactggtggactcaacagtctgcaagtaactttaaggagcaatcccagtg | |
| | catggtgtatcagtatggaaacttttcctgggacctggcaggtggacagcaccttaatgg | |
| | aattaatacactgggagaaaacattgctgataatggaggtcttggtcaagcatacagag | |
| | cctatcagaattatattaaaaagaatggcgaagaaaaattacttcctggacttgacctaa | |
| | atcacaaacaactatttttcttgaactttgcacaggtgtggtgtggaacctataggccaga | |
| | gtatgcggttaactccattaaaacagatgtgcacagtccaggcaatttcaggattattggg | |
| | actttgcagaactctgcagagttttcagaagcctttcactgccgcaagaattcatacatga | |
| | atccagaaaagaagtgccgggtttgg | |
| Right Homology | tagagtcagcttacatccctgagcaggaaagtttacccatgaagattggtgggattttttgt | 6 |
| Arm | ttgtttgttttgttttgtttgttgtttgttgtttgtttttttgccactaattttagtattcattctgca | |
| | ttgctagataaaagctgaagttactttatgtttgtcttttaatgcttcattcaatattgacatttgtag | |
| | ttgagcggggggtttggtttgctttggtttatattttttcagttgtttgtttttgcttgttatattaag | |
| | cagaaatcctgcaatgaaaggtactatatttgctagactctagacaagatattgtacataaaag | |
| | aatttttttgtctttaaatagatacaaatgtctatcaactttaatcaagttgtaacttatattgaa | |
| | gacaatttgatacataataaaaaattatgacaatgtcctggactggtttttgttttgtttaattat | |
| | tttgagagctatttggtttgcaaatgtctttcaactcctattaactccacctgcatttttttttttttt | |
| | taagacggagtttcgctcttgttgcccaggctggagtgcaatggcccgatcttggctcacc | |
| | acagcctccacctcctgggttcaaacaattctcctgcctcagcttcctgagtagctgggatt | |
| | acaggcatgcaccaccacacccggctaattttgtattittagtagagacggggtttctccgt | |
| | gttggtcaggctggtctcgaactcccaacctcagttgatccgcccgcctcagcctcccaa | |
| | agtgctgggattacaggcgtgagccaccgtgcccggccccacctgcatttatatgagaa | |
| | gtagattgggaggttgctcaccccagtttactaatgaagaaatctagatgtggagagatt | |
| | aaaggattctgatccaaagtcacacaggtgtcaacagaggagtcagcaccagcacct | |
| | ggtcctcagacgctcattcagcagtggggtatatcttggggatat | |
| Left Homology | tcccccacccccgcctgcctctgccctcttgccagactgcccccaaagagagaatcgc | 7 |
| Arm | ctcctttcttagtggttatttgcacccttcaccggcatcttaagaaaggcatgaagcaccttt | |
| | cagcaaaatatacgacaataccttcttttttttctttttttctttttttgagacgaagtttcactcttg | |
| | tcgcccaggctggagtgcagtggtgtgatctcgactcactgcaacctccgcctctctggg | |
| | ttcaagcaattatccagcctcagcctcccgagtagctgggactacaggcgtgtgccacc | |
| | atgcccagctgattatttttagtagagatggggtttcaccatgttggtcaggctggtctcgaa | |
| | ctcctgacctcaggtaatctgcctgccttggcctcccaaagtgctgggattacaggcgtg | |
| | agccactgtgcccagccaagatataacaatacgttttaaaatagaatattaacattttggg | |
| | aggccgaggcaggcggatcgcaccacagcactctagcctaagcaacagagcaaga | |
| | ctccatctcaaaagaaaaaaaaaagagtattaaataacatacacacaaagacaaat | |
| | gcaaaagtactaacctgtatggactaggactataggaaaacctggaatttgagcattaa | |
| | atttggctctgagcttactttcccaccctgaaagttctccaaggaaaatgcttgtgcagctg | |
| | actgccaacccccccagattcctcacgtcccgcacccttctcattatctggcctgcatctct | |
| | cctgcttcagcaccttctgactcgtttggcctcctggatgccattctccttgtcatttttcttgga | |
| | ctgggtgacccatgtctctccctttccctcagccttccttcagatcaaaccaccctgatcctc | |
| | atgtttcttcctatctcctagatatttggcatgatcttcagtatgatcttgtgctgtgctatccgc | |
| | aggaaccgcgagatggtc | |
| gRNA | gctgactctagaccatctcgcgg | 8 |

TABLE 3:
Shows non-limiting examples of constructs used herein, and portions thereof.

| | | SEQ ID |
|---|---|---|
| | Sequence | NO: |
| CD9 isoform 1: | MPVKGGTKCIKYLLFGFNFIFWLAGIAVLAIGLWLRFDSQTKSI | 9 |
| P2A: NEP | FEQETNNNNSSFYTGVYILIGAGALMMLVGFLGCCGAVQESQ | |
| | CMLGLFFGFLLVIFAIEIAAAIWGYSHKDEVIKEVQEFYKDTYN | |
| | KLKTKDEPQRETLKAIHYALNCCGLAGGVEQFISDICPKKDVL | |
| | ETFTVKSCPDAIKEVFDNKFHIIGAVGIGIAVVMIFGMIFSMILC | |
| | CAIRRNREMVATNFSLLKQAGDVEENPGPMGKSESQMDITDI | |
| | NTPKPKKKQRWTPLEISLSVLVLLLTIIAVTMIALYATYDDGICKS | |
| | SDCIKSAARLIQNMDATTEPCTDFFKYACGGWLKRNVIPETSS | |
| | RYGNFDILRDELEVVLKDVLQEPKTEDIVAVQKAKALYRSCINE | |
| | SAIDSRGGEPLLKLLPDIYGWPVATENWEQKYGASWTAEKAIA | |
| | QLNSKYGKKVLINLFVGTDDKNSVNHVIHIDQPRLGLPSRDYY | |
| | ECTGIYKEACTAYVDFMISVARLIRQEERLPIDENQLALEMNKV | |
| | MELEKEIANATAKPEDRNDPMLLYNKMTLAQIQNNFSLEINGKP | |
| | FSWLNFTNEIMSTVNISITNEEDVVVYAPEYLTKLKPILTKYSAR | |
| | DLQNLMSWRFIMDLVSSLSRTYKESRNAFRKALYGTTSETATW | |
| | RRCANYVNGNMENAVGRLYVEAAFAGESKHVVEDLIAQIREVF | |
| | IQTLDDLTWMDAETKKRAEEKALAIKERIGYPDDIVSNDNKLNN | |
| | EYLELNYKEDEYFENIIQNLKFSQSKQLKKLREKVDKDEWISG | |
| | AAVVNAFYSSGRNQIVFPAGILQPPFFSAQQSNSLNYGGIGMV | |
| | IGHEITHGFDDNGRNFNKDGDLVDWWTQQSASNFKEQSQCM | |
| | VYQYGNFSWDLAGGQHLNGINTLGENIADNGGLGQAYRAYQ | |
| | NYIKKNGEEKLLPGLDLNHKQLFFLNFAQVWCGTYRPEYAVNS | |
| | IKTDVHSPGNFRIIGTLQNSAEFSEAFHCRKNSYMNPEKKCRV | |
| | W* | |
| CD9 isoform 2: | MMLVGFLGCCGAVQESQCMLGLFFGFLLVIFAIEIAAAIWGYS | 10 |
| P2A: NEP | HKDEVIKEVQEFYKDTYNKLKTKDEPQRETLKAIHYALNCCG | |
| | LAGGVEQFISDICPKKDVLETFTVKSCPDAIKEVFDNKFHIIGA | |
| | VGIGIAVVMIFGMIFSMILCCAIRRNREMVATNFSLLKQAGDVE | |
| | ENPGPMGKSESQMDITDINTPKPKKKQRWTPLEISLSVLVLLLT | |
| | IIAVTMIALYATYDDGICKSSDCIKSAARLIQNMDATTEPCTDFFK | |
| | YACGGWLKRNVIPETSSRYGNFDILRDELEVVLKDVLQEPKTE | |
| | DIVAVQKAKALYRSCINESAIDSRGGEPLLKLLPDIYGWPVATE | |
| | NWEQKYGASWTAEKAIAQLNSKYGKKVLINLFVGTDDKNSVN | |
| | HVIHIDQPRLGLPSRDYYECTGIYKEACTAYVDFMISVARLIRQE | |
| | ERLPIDENQLALEMNKVMELEKEIANATAKPEDRNDPMLLYNK | |
| | MTLAQIQNNFSLEINGKPFSWLNFTNEIMSTVNISITNEEDVVV | |
| | YAPEYLTKLKPILTKYSARDLQNLMSWRFIMDLVSSLSRTYKES | |
| | RNAFRKALYGTTSETATWRRCANYVNGNMENAVGRLYVEAAF | |
| | AGESKHVVEDLIAQIREVFIQTLDDLTWMDAETKKRAEEKALAI | |
| | KERIGYPDDIVSNDNKLNNEYLELNYKEDEYFENIIQNLKFSQS | |
| | KQLKKLREKVDKDEWISGAAVVNAFYSSGRNQIVFPAGILQPP | |
| | FFSAQQSNSLNYGGIGMVIGHEITHGFDDNGRNFNKDGDLVD | |
| | WWTQQSASNFKEQSQCMVYQYGNFSWDLAGGQHLNGINTL | |
| | GENIADNGGLGQAYRAYQNYIKKNGEEKLLPGLDLNHKQLFFL | |
| | NFAQVWCGTYRPEYAVNSIKTDVHSPGNFRIIGTLQNSAEFSE | |
| | AFHCRKNSYMNPEKKCRVW* | |
| CD9 isoform 1: | MPVKGGTKCIKYLLFGFNFIFWLAGIAVLAIGLWLRFDSQTKSI | 11 |
| P2A: SignalPeptide: | FEQETNNNNSSFYTGVYILIGAGALMMLVGFLGCCGAVQESQ | |
| sNEP | CMLGLFFGFLLVIFAIEIAAAIWGYSHKDEVIKEVQEFYKDTYN | |
| | KLKTKDEPQRETLKAIHYALNCCGLAGGVEQFISDICPKKDVL | |
| | ETFTVKSCPDAIKEVFDNKFHIIGAVGIGIAVVMIFGMIFSMILC | |
| | CAIRRNREMVATNFSLLKQAGDVEENPGPMGAASGRRGPGL | |
| | LLPLPLLLLLPPQPALAYDDGICKSSDCIKSAARLIQNMDATTEP | |
| | CTDFFKYACGGWLKRNVIPETSSRYGNFDILRDELEVVLKDVL | |
| | QEPKTEDIVAVQKAKALYRSCINESAIDSRGGEPLLKLLPDIYG | |
| | WPVATENWEQKYGASWTAEKAIAQLNSKYGKKVLINLFVGTD | |
| | DKNSVNHVIHIDQPRLGLPSRDYYECTGIYKEACTAYVDFMISV | |
| | ARLIRQEERLPIDENQLALEMNKVMELEKEIANATAKPEDRNDP | |
| | MLLYNKMTLAQIQNNFSLEINGKPFSWLNFTNEIMSTVNISITN | |
| | EEDVVVYAPEYLTKLKPILTKYSARDLQNLMSWRFIMDLVSSLS | |
| | RTYKESRNAFRKALYGTTSETATWRRCANYVNGNMENAVGRL | |
| | YVEAAFAGESKHVVEDLIAQIREVFIQTLDDLTWMDAETKKRA | |
| | EEKALAIKERIGYPDDIVSNDNKLNNEYLELNYKEDEYFENIIQN | |
| | LKFSQSKQLKKLREKVDKDEWISGAAVVNAFYSSGRNQIVFPA | |
| | GILQPPFFSAQQSNSLNYGGIGMVIGHEITHGFDDNGRNFNKD | |
| | GDLVDWWTQQSASNFKEQSQCMVYQYGNFSWDLAGGQHL | |
| | NGINTLGENIADNGGLGQAYRAYQNYIKKNGEEKLLPGLDLNH | |
| | KQLFFLNFAQVWCGTYRPEYAVNSIKTDVHSPGNFRIIGTLQN | |
| | SAEFSEAFHCRKNSYMNPEKKCRVW* | |
| CD9 isoform 2: | MMLVGFLGCCGAVQESQCMLGLFFGFLLVIFAIEIAAAIWGYS | 12 |
| P2A: SignalPeptide: | HKDEVIKEVQEFYKDTYNKLKTKDEPQRETLKAIHYALNCCG | |
| sNEP | LAGGVEQFISDICPKKDVLETFTVKSCPDAIKEVFDNKFHIIGA | |
| | VGIGIAVVMIFGMIFSMILCCAIRRNREMVATNFSLLKQAGDVE | |
| | ENPGPMGAASGRRGPGLLLPLPLLLLLPPQPALAYDDGICKSS | |
| | DCIKSAARLIQNMDATTEPCTDFFKYACGGWLKRNVIPETSSR | |
| | YGNFDILRDELEVVLKDVLQEPKTEDIVAVQKAKALYRSCINES | |
| | AIDSRGGEPLLKLLPDIYGWPVATENWEQKYGASWTAEKAIAQ | |
| | LNSKYGKKVLINLFVGTDDKNSVNHVIHIDQPRLGLPSRDYYE | |
| | CTGIYKEACTAYVDFMISVARLIRQEERLPIDENQLALEMNKVM | |
| | ELEKEIANATAKPEDRNDPMLLYNKMTLAQIQNNFSLEINGKPF | |
| | SWLNFTNEIMSTVNISITNEEDVVVYAPEYLTKLKPILTKYSARD | |
| | LQNLMSWRFIMDLVSSLSRTYKESRNAFRKALYGTTSETATWR | |
| | RCANYVNGNMENAVGRLYVEAAFAGESKHVVEDLIAQIREVFI | |
| | QTLDDLTWMDAETKKRAEEKALAIKERIGYPDDIVSNDNKLNN | |
| | EYLELNYKEDEYFENIIQNLKFSQSKQLKKLREKVDKDEWISG | |
| | AAVVNAFYSSGRNQIVFPAGILQPPFFSAQQSNSLNYGGIGMV | |
| | IGHEITHGFDDNGRNFNKDGDLVDWWTQQSASNFKEQSQCM | |
| | VYQYGNFSWDLAGGQHLNGINTLGENIADNGGLGQAYRAYQ | |
| | NYIKKNGEEKLLPGLDLNHKQLFFLNFAQVWCGTYRPEYAVNS | |
| | IKTDVHSPGNFRIIGTLQNSAEFSEAFHCRKNSYMNPEKKCRV | |
| | W* | |
| CD9 isoform 1 | MPVKGGTKCIKYLLFGFNFIFWLAGIAVLAIGLWLRFDSQTKSIF | 13 |
| | EQETNNNNSSFYTGVYILIGAGALMMLVGFLGCCGAVQESQC | |
| | MLGLFFGFLLVIFAIEIAAAIWGYSHKDEVIKEVQEFYKDTYNKL | |
| | KTKDEPQRETLKAIHYALNCCGLAGGVEQFISDICPKKDVLETF | |
| | TVKSCPDAIKEVFDNKFHIIGAVGIGIAVVMIFGMIFSMILCCAIR | |
| | RNREMV | |
| CD9 isoform 2 | MMLVGFLGCCGAVQESQCMLGLFFGFLLVIFAIEIAAAIWGYSH | 14 |
| | KDEVIKEVQEFYKDTYNKLKTKDEPQRETLKAIHYALNCCGLA | |
| | GGVEQFISDICPKKDVLETFTVKSCPDAIKEVFDNKFHIIGAVGI | |
| | GIAVVMIFGMIFSMILCCAIRRNREMV | |
| P2A | ATNFSLLKQAGDVEENPGP | 15 |
| Signal Peptide | MGAASGRRGPGLLLPLPLLLLLPPQPALA | 16 |

*indicates a STOP codon; As used herein a “secretion signal” and “signal peptide” may be used interchangeably.

### Example 4. Expression of Neprilysin by Designed iPSC-Derived Microglia In Vitro

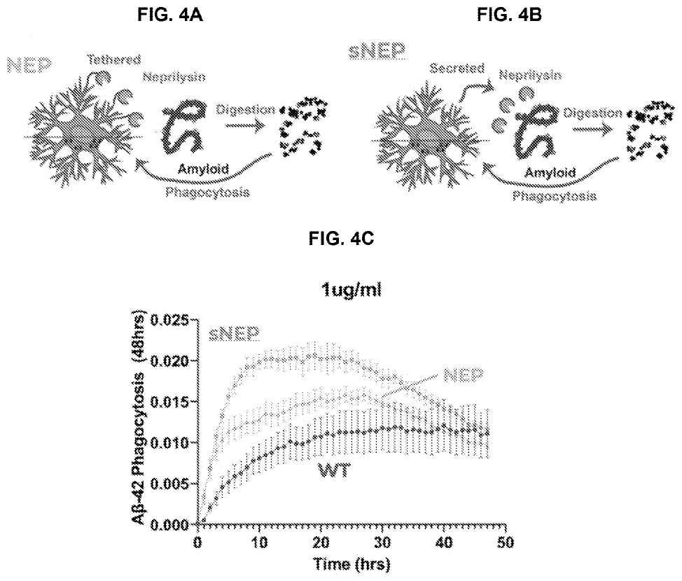

The digestion and phagocytosis properties of the designed iPSC-derived microglia were determined. The phagocytic activity of isogenic WT, CD9:NEP (FIG. 4A), and CD9:sNEP (FIG. 4B) iPS-derived microglia was examined using the IncuCyte S3 Live-Cell Analysis System (Sartorius). iPS-Microglia were plated on Matrigel-coated 96-well plates at 40K cells per well (6 wells per line). 45 min after plating, wells were plated with 500 ng/ml, 1 ug/ml, and 2 ug/ml 488-labeled fibrillized-Aβ (1-42) (Anaspec) for recording. Using IncuCyte 2020B software, image masks for fluorescent signal (phagocytosis of 488-labeled fibrillized-Aβ) were normalized to cell body area. Aβ shown in FIG. 40, significant phagocytosis by cells expressing and secreting neprilysin was observed (FIG. 41B). Moreover, higher levels of phagocytosis were observed in cells expressing tethered neprilysin over wild-type microglia (FIGS. 4A and 4C). These studies demonstrate that engineered microglia are capable of expressing the therapeutic payload neprilysin and that expressed neprilysin increases the phagocytosis of Aβ polymers.

### Example 5. Demonstrated Expression of Neprilysin by Designed iPSC-Derived Microglia In Vivo

As used herein, the term “xMGs” refers to cells having been xenotransplanted or re-isolated from the brain. The term “iMGL” refers to the cells in vitro or prior to any transplantation.

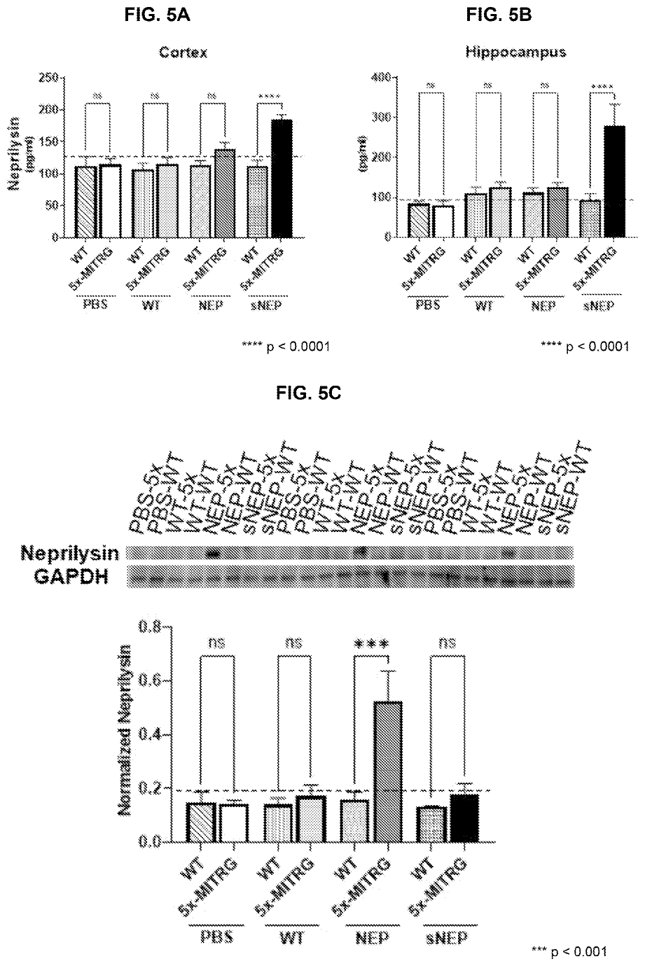

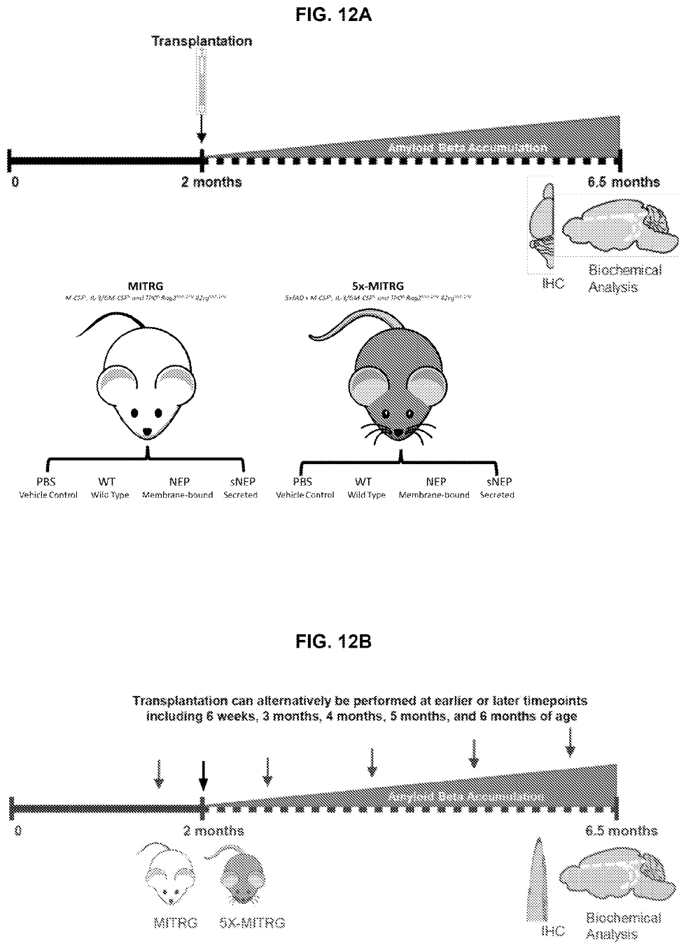

The activity of engrafted xMGs according to the present disclosure was explored in a series of engraftment studies in MITRG and 5×-MITRG mice, the design of which is shown schematically in FIG. 12A. Briefly, Human iPSC-derived microglial progenitors were transplanted into the hippocampus and overlying cortex of 2-month-old WT and 5×-MITRG mice. At 6.5 months of age, mice were sacrificed, and the hippocampus and cortex were microdissected and examined using an ELISA that detects both endogenous murine neprilysin and human neprilysin and is more sensitive to soluble, secreted NEP than membrane-bound NEP. As shown in FIG. 5A-5B, xMGs of this disclosure specifically expressed both soluble (sNEP) and membrane-bound (NEP) (FIG. 5C) species when transplanted into 5×-MITRG but not WT-MITRG mice, with the soluble species detectable over background in soluble extracts of both cortex and hippocampus and the membrane-bound species detectable in soluble extracts from hippocampus samples sNEP levels were significantly increased only in 5×-MITRG mice, not WT mice, as a result of CD9-regulated expression of sNEP which is specifically induced by proximity to amyloid pathology (see FIGS. 1A-1P and FIG. 2A-2H).

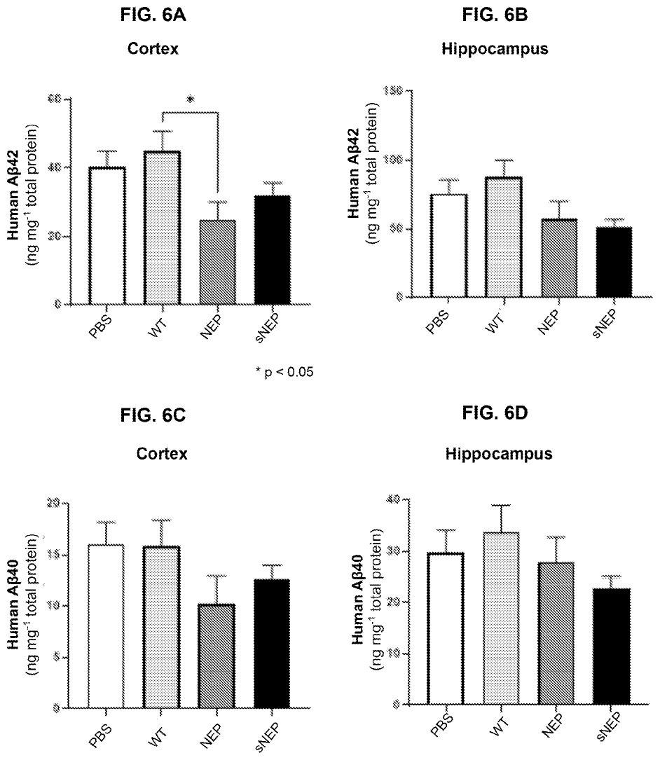

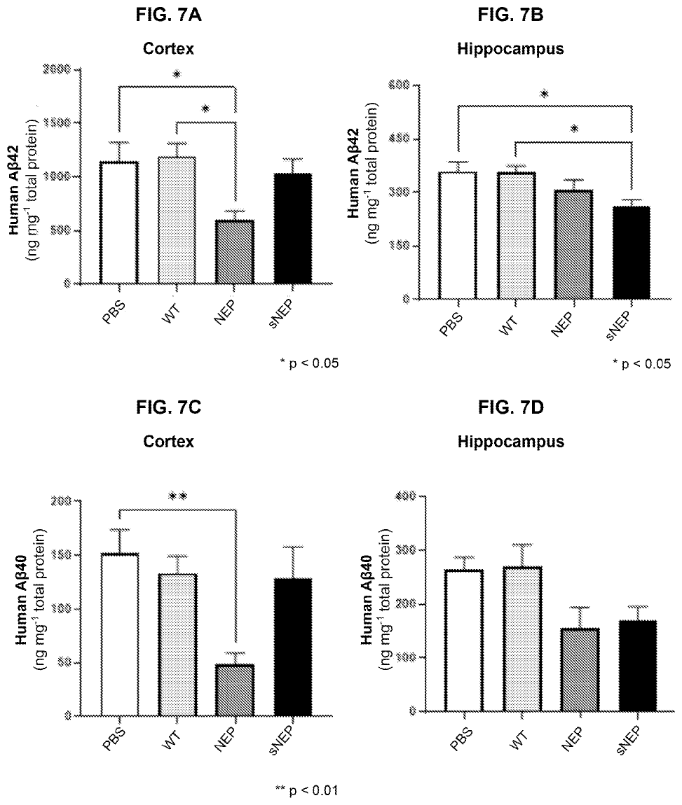

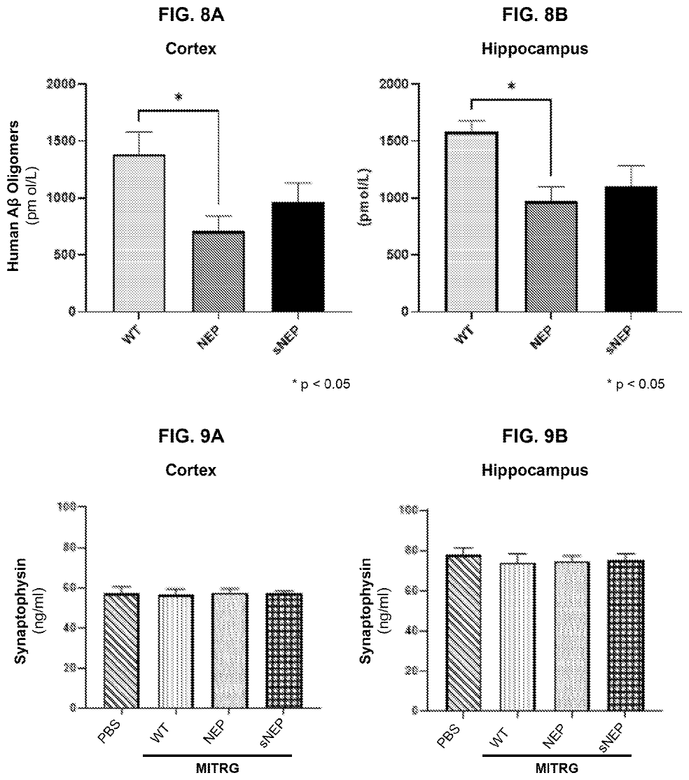

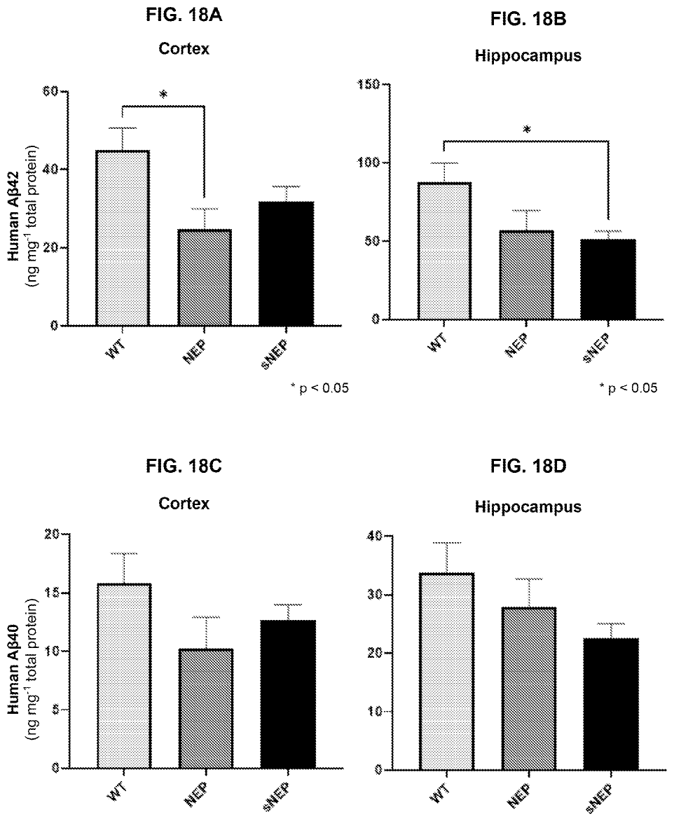

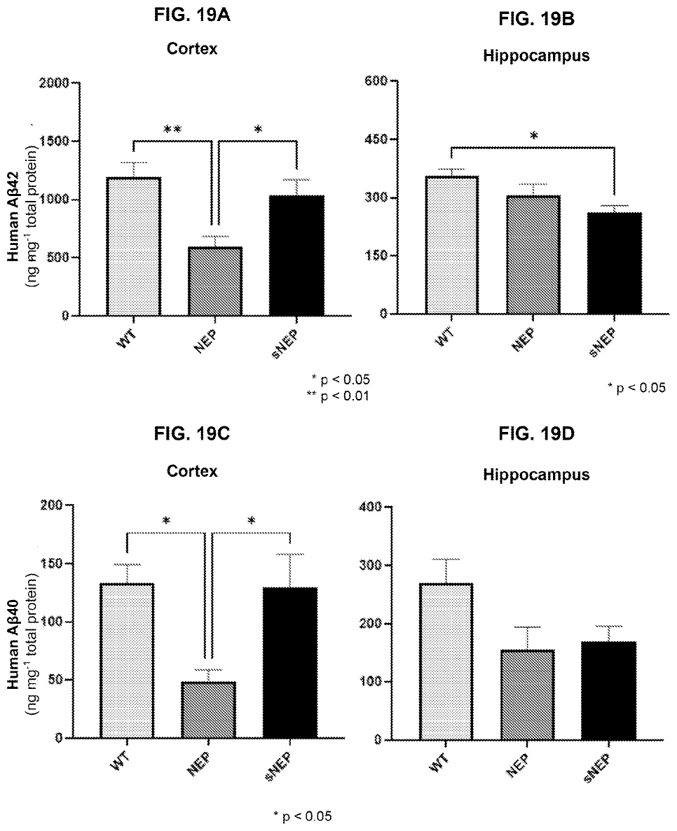

The inventors observed statistically significant reductions in Human Aβ-42 and Aβ-40 peptide in soluble and insoluble fractions of cortex and hippocampus from 5×-MITRG mice treated with either NEP- or sNEP-expressing iMGLs, as shown in FIGS. 6A-6D, FIGS. 7A-7D, FIGS. 18A-18D, and FIGS. 19A-19D. NEP-expressing microglia significantly reduced soluble Aβ-42 levels within the cortex (ANOVA with Tukey's post hoc test, p<0.05). Additional trends toward reduced Aβ are observed in the hippocampus with NEP and sNEP expression. A statistically significant reduction in human Aβ oligomers in soluble hippocampus and cortex fractions was also observed in NEP treated animals by MSD assay, as shown in FIG. 8A-8B. These measurements also suggested a trend toward reduction in the sNEP treated animals but did not reach statistical significance in the samples examined. Nonetheless, in aggregate, these data indicate that both NEP- and sNEP-expressing iMGLs are capable of reducing at least some AD-associated Aβ species in vivo.

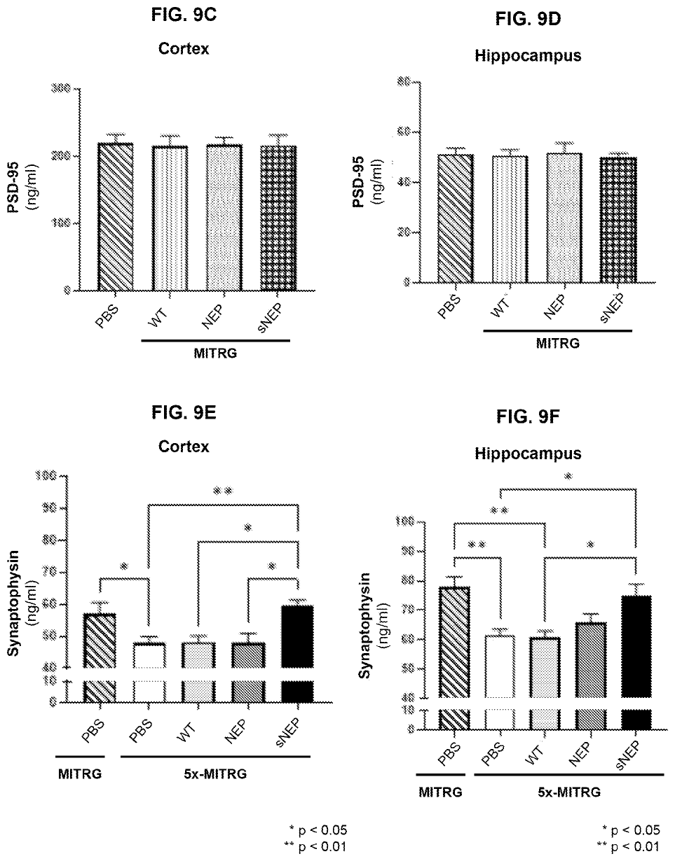

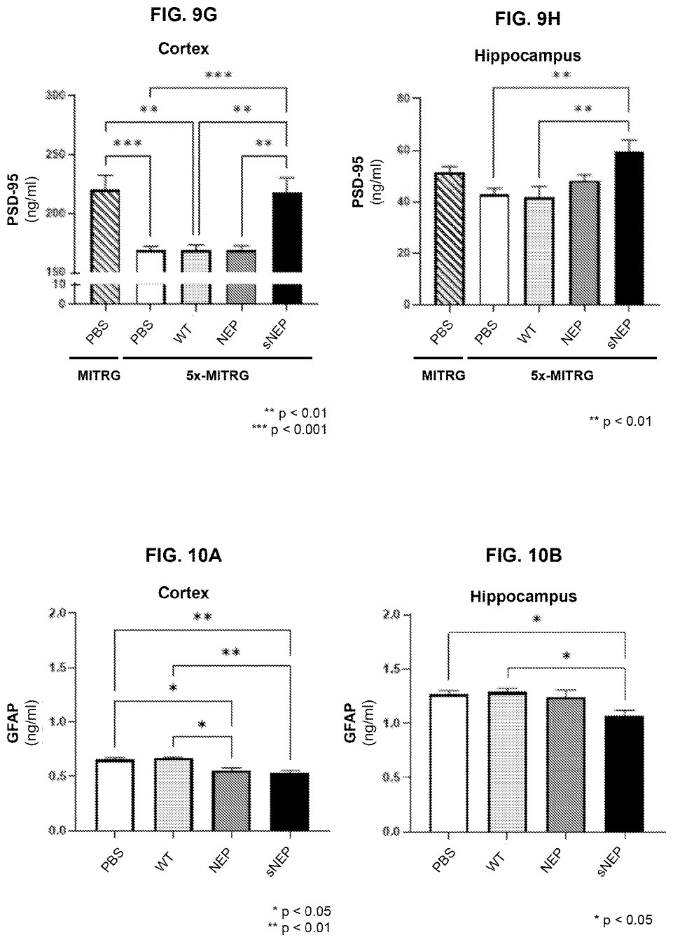

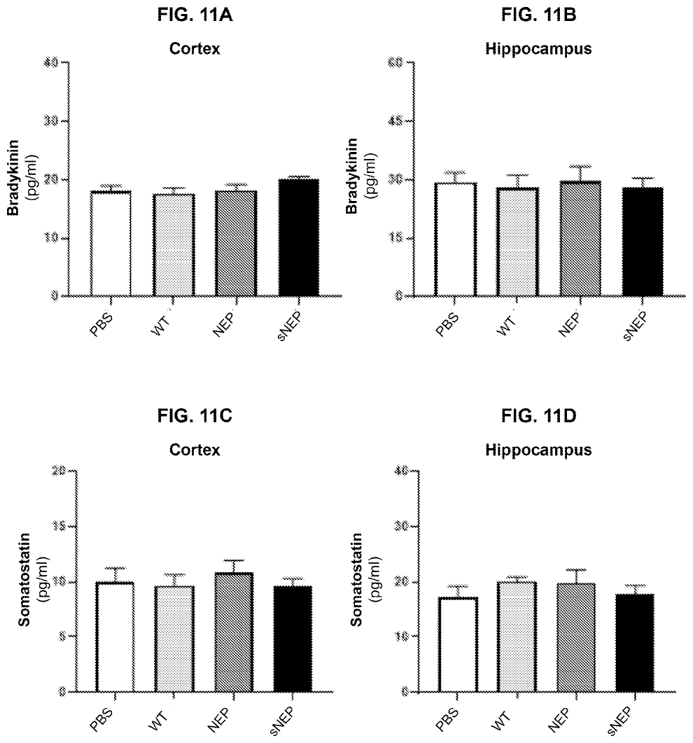

Reductions in Aβ species were complemented by reductions in phenotypic signals associated with AD. As shown in FIG. 9A-9H, sNEP-treated 5×-MITRG mice showed increases in pre- and post-synaptic markers that suggested a reduction or reversal of the synapse loss seen in untreated 5×-fAD mice and human patients. Reductions in GFAP expression, a marker of astrogliosis observed, were also seen in both NEP- and sNEP-treated 5×MITRG mice, as shown in FIG. 10A-10B. Taken together, these data indicate that iMGLs are capable of reducing or correcting AD-associated cellular and molecular phenotypes in vivo.

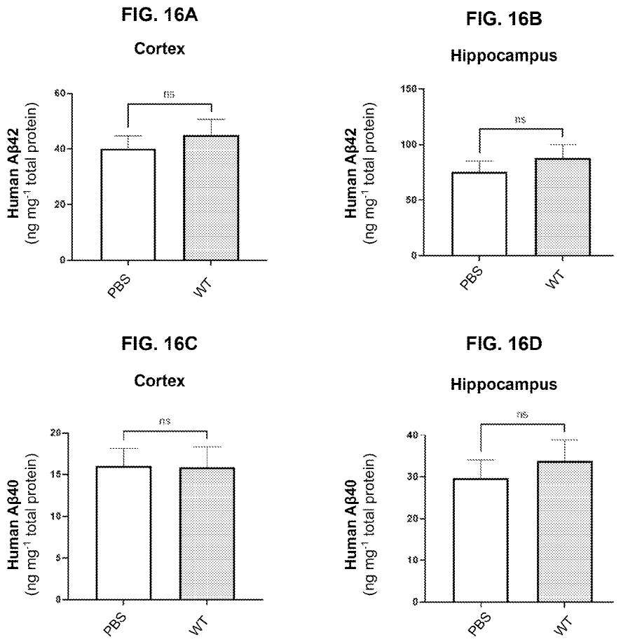

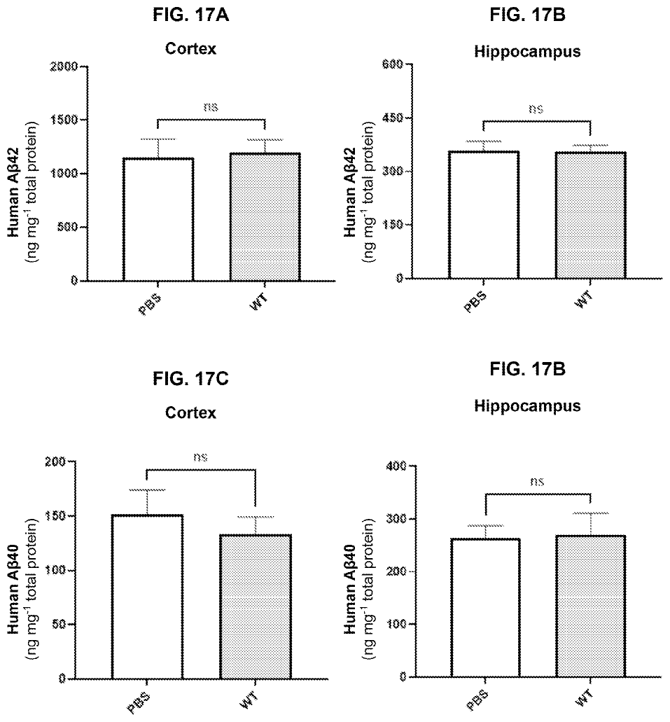

Crucially, the reductions in AD-associated phenotypic signals shown in FIGS. 5A-10B were not accompanied by significant off-target effects of Neprilysin expression (FIGS. 16A-16D and FIGS. 17A-17D). As shown in FIGS. 11A-11D, the levels of two non-Aβ substrates of Neprilysin, Bradykinin, and Somatostatin, were substantially constant in cortex and hippocampus samples from sham-treated (PBS), WT, NEP, and sNEP-treated 5×MITRG mice, indicating that systemic Neprilysin activity is not increased to a level that would cause off-target degradation by administration of NEP- or sNEP-expressing iMGLs according to this disclosure.

Taken together, these data provide strong evidence that the systems and methods of the present disclosure deliver pharmaceutically effective doses of therapeutic payloads with high specificity to sites of AD pathology and that the systems and methods of the present disclosure may be effective in the treatment, palliation, or prevention of amyloid beta-related pathologies, e.g., Alzheimer's-related pathologies.

### Example 6. Amyloid Pathology Induces CD9 Expression within Human Microglia, which Leads to Highly Localized Induction of Neprilysin

Two-month-old 5×-MITRG mice were transplanted into the hippocampus and overlying cortex with either PBS vehicle, unmodified human microglial progenitors, CD9-NEP modified human microglial progenitors or CD9-sNEP modified human microglial progenitors. At 6.5 months of age mice were sacrificed and brains examined using immunohistochemistry and confocal microscopy. To determine whether neprilysin expression is induced in plaque-associated human microglia under control of the CD9 promoter sections were labeled for Ku80 to detect human microglial nuclei, Amylo-Glo to detect beta-amyloid plaques, CD9 and neprilysin.

As shown, Ku80+ human microglial nuclei are observed scattered throughout the tissue. However, CD9 expression is only upregulated in human Ku80+ microglia that are adjacent to Amylo-Glo positive amyloid plaques. Furthermore, Neprilysin expression is highly correlated with CD9 expression and only observed in microglia that are adjacent to plaques. These immunohistochemical images therefore demonstrate that CD9-regulated expression of neprilysin is highly localized to only plaque-adjacent human microglia. These data therefore demonstrate that human microglia modified to introduce the NEP and sNEP sequences under control of the CD9 promoter are functioning as specifically designed.

As shown, Ku80+ human microglial nuclei are observed scattered throughout the tissue. However, CD9 expression is only upregulated in human Ku80+ microglia that are adjacent to Amylo-Glo positive amyloid plaques. Furthermore, Neprilysin expression is highly correlated with CD9 expression and only observed in microglia that are adjacent to plaques. These immunohistochemical images therefore demonstrate that CD9-regulated expression of neprilysin is highly localized to only plaque-adjacent human microglia. These data therefore demonstrate that human microglia modified to introduce the NEP and sNEP sequences under control of the CD9 promoter are functioning as specifically designed.

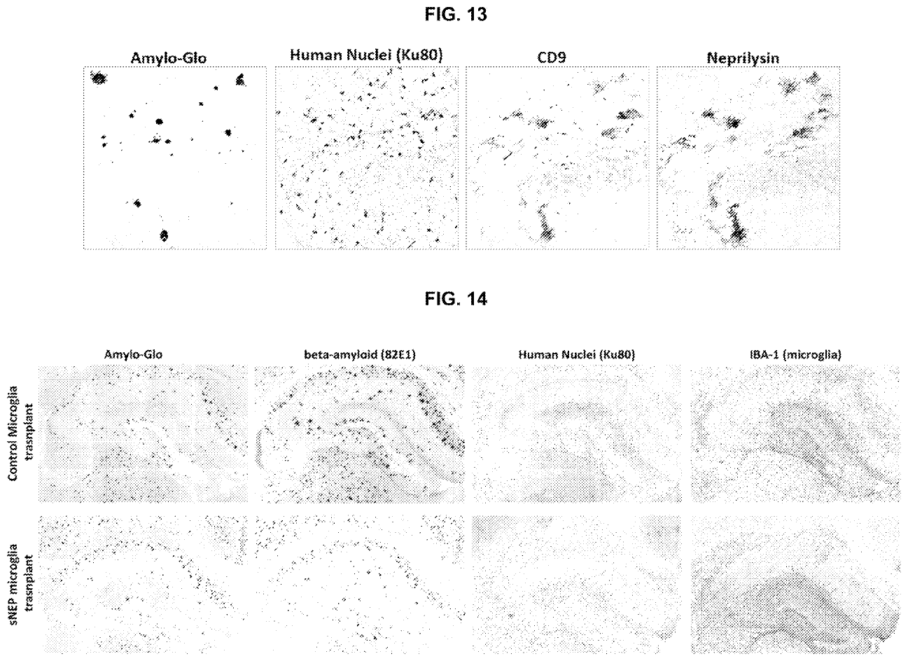

Results depicted in FIG. 13 showed that CD9 expression is induced in human microglia by amyloid pathology, leading to highly localized induction of neprilysin. Ku80 was used to visualize the nuclei of transplanted human microglia. Importantly, CD9 expression and consequent neprilysin induction were observed only in human microglia located adjacent to Amylo-Glo positive beta-amyloid plaques.

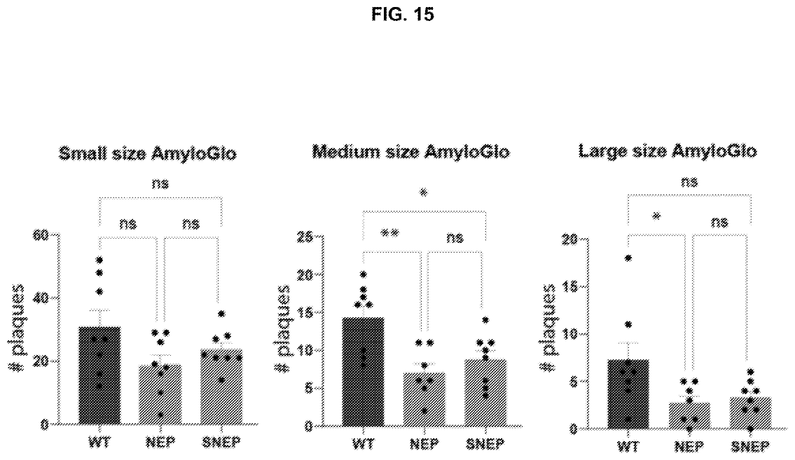

Additionally, FIG. 14 demonstrated that hippocampal Aβ pathology can be reduced by sNEP-expressing human microglia in vivo, indicating the therapeutic potential of using genetically modified human microglia for the treatment of Alzheimer's disease. In addition, as shown in FIG. 15, the numbers of medium and large AmyloGlo positive plaques were significantly reduced in vivo with the induction of neprilysin, further highlighting the potential of this approach as a therapeutic strategy for Alzheimer's disease.

### Example 7: Use of the Modified Cells Described Herein to Prevent Alzheimer's Disease Path

A 45-year-old subject, who has undergone a genetic test and discovered that she has an increased risk of developing Alzheimer's disease, consults with her physician to seek professional advice. The physician suggests a novel therapy involving iPSC-derived Human Microglia Cells (iMGLs) that express and release a therapeutic molecule (e.g., neprilysin) when in proximity to Alzheimer's-related pathology (e.g., alpha-beta plaques). The doctor explains that the therapeutic molecule (e.g., neprilysin) is expected to prevent the formation of any Aβ pathology. The subject consents to undergo the therapy but is first required to take a series of cognitive tests. After receiving the treatment, the subject is able to successfully complete yearly cognitive tests during check-ups. No adverse side effects were reported.

### Example 8: Use of the Modified Cells Described Herein to Ameliorate Alzheimer's Disease Pathology

A 65-year-old individual has recently been diagnosed with Alzheimer's Disease after undergoing numerous cognitive tests and biopsies revealing beta-amyloid plaques. Together with their family, they consult with a doctor to explore possible treatment options. The doctor recommends a therapeutic composition comprising modified iMGLs cells. The doctor explains that a sample of the subject's cells will be retrieved and modified to incorporate a therapeutic molecule with neprilysin activity. After modification, the cells will be administered to the subject's hippocampus. After consenting to the treatment, the subject undergoes two surgeries, one to retrieve the cells and another to administer the modified cells. Subsequently, the subject undergoes regular cognitive tests and biopsies to evaluate the effectiveness of the treatment. Six months after treatment, the subject's cognitive test results show improvement, and their biopsies show a reduction in amyloid plaques. The doctor plans to monitor the subject every six months for the next 5 years and once a year thereafter. No adverse side effects have been reported.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of,” and as such the written description requirement for claiming one or more embodiments of the present invention using the phrase “consisting essentially of” or “consisting of” is met.

What is claimed is:

1. A human induced microglia-like (iMGL) cell that expresses and presents a therapeutic molecule comprising membrane-bound neprilysin or expresses and secretes a therapeutic molecule comprising secreted neprilysin.

2. A human induced microglia-like (iMGL) cell that expresses and presents or expresses and secretes a therapeutic molecule comprising: membrane-bound neprilysin, secreted neprilysin, TREM2, APOE, LRP1, insulin degrading enzyme, endothelin-converting enzyme, plasminogen activator, angiotensin-converting enzyme, or a matrix metalloproteinase.

3. The cell of claim 1 or 2, wherein the promoter is configured to activate transcription of the therapeutic molecule upon (i) proximity of the cell to (i-amyloid (Aβ) peptide plaques, soluble Aβ monomers, insoluble Aβ monomers, Aβ oligomers, pyroglutamate Aβ, protofibrils, or fibrils comprising Aβ of or a fragment thereof; or (ii) contact of the cell with β-amyloid (Aβ) peptide plaques, soluble Aβ monomers, insoluble Aβ monomers, Aβ oligomers, pyroglutamate Aβ, protofibrils, or fibrils comprising Aβ or a fragment thereof.

4. The cell of claim 3, wherein the cell expresses P2RY12 and TREM2.

5. The cell of claim 1 or 2, wherein the cell comprises a nucleic acid sequence encoding the therapeutic molecule; and a promoter selected from a CD9 gene promoter, a LGALS3 gene promoter, an HLA-DRB gene promoter, or a CD11c gene promoter, wherein the promoter is operatively linked to the nucleic acid sequence encoding the therapeutic molecule.

6. The cell of claim 5, wherein the promoter comprises the CD9 gene promoter.

7. The cell of claim 5, wherein the promoter comprises the LGALS3 gene promoter.

8. The cell of claim 5, wherein the promoter comprises the HLA-DRB gene promoter.

9. The cell of claim 5, wherein the promoter comprises the CD11c gene promoter.

10. The cell of any one of claims 6-9, wherein the therapeutic molecule comprises membrane-bound neprilysin or secreted neprilysin.

11. The cell of any one of claims 6-9, wherein the therapeutic molecule comprises one of SEQ ID NO: 1, SEQ ID NO: 2, or SEQ ID NO: 3.

12. The cell of any one of claims 6-9, wherein the therapeutic molecule comprises TREM2.

13. The cell of any one of claims 6-9, wherein the therapeutic molecule comprises insulin degrading enzyme.

14. The cell of any one of claims 1-13, wherein the nucleic acid sequence is inserted into the genome of the cell.

15. The cell of claim 14, wherein the nucleic acid sequence is inserted downstream of a locus controlled by the promoter such that the nucleic acid sequence is in-frame with a coding sequence in an exon of the locus.

16. The cell of claim 14, wherein the nucleic acid sequence is inserted within the locus controlled by the promoter such that the nucleic acid sequence is joined, inframe, with at least part of the coding sequence of the locus.

17. The cell of any one of claims 15-16, wherein a first polynucleotide encoding a protease cleavage site, a ribosomal skipping sequence, or a self-cleaving peptide is inserted between the coding sequence of the exon and the nucleic acid sequence encoding the therapeutic protein.

18. The cell of claim 17, wherein the self-cleaving peptide is P2A.

19. The cell of claim 17, wherein a second polynucleotide encoding a secreted peptide signal sequence is inserted at the 5′ end of the nucleic acid sequence encoding the therapeutic protein.

20. The cell of any one of claims 1-19, wherein the therapeutic protein affects one or more amyloid-beta (Aβ)-related pathologies.

21. The cell of claim 20, wherein the Aβ-related pathology comprises β-amyloid (Aβ) peptide plaques, soluble Aβ monomers, insoluble Aβ monomers, Aβ oligomers, pyroglutamate Aβ, protofibrils, or fibrils comprising Aβ of varying lengths.

22. The cell of any one of claims 20-21, wherein the therapeutic molecule reduces the amount of Aβ peptide in Aβ peptide plaques in a brain of an individual.

23. The cell of any one of claims 20-21, wherein the therapeutic molecule reduces the size or number of soluble Aβ monomers, insoluble Aβ monomers, Aβ oligomers, pyroglutamate Aβ, protofibrils, or fibrils comprising Aβ of varying lengths.

24. The cell of any one of claims 20-21 wherein the therapeutic molecule enhances amyloid proteolysis.

25. The cell of any one of claims 20-21, wherein the therapeutic molecule enhances microglial phagocytosis of amyloid beta.

26. A modified cell that expresses and presents or secretes a therapeutic molecule when the cell is proximal to or in contact with amyloid-beta (Aβ)-related pathology, wherein the therapeutic molecule reduces or eliminates an Aβ-related pathology phenotype or ameliorates a symptom of the Aβ-related pathology.

27. A modified cell that expresses and presents or secretes a therapeutic molecule when the cell is proximal to, or in contact with amyloid-beta (Aβ)-related pathology, wherein the therapeutic molecule reduces or eliminates at least one aspect of the Aβ-related pathology.

28. A modified cell that expresses and presents or secretes a therapeutic molecule when the cell is proximal to, or in contact with an amyloid-beta (Aβ) aggregate, wherein the therapeutic molecule reduces or eliminates an amyloid beta-related pathology or ameliorates a symptom thereof.

29. A modified cell that expresses and presents or secretes a therapeutic molecule when the cell is proximal to, or in contact with an amyloid-beta (Aβ) aggregate, plaque, oligomer, or fibril, wherein the therapeutic molecule reduces or eliminates an amyloid beta-related pathology or ameliorates a symptom thereof.

30. The cell of any one of claims 26-29, wherein the amyloid beta-related pathology phenotype comprises a phenotype selected from the group consisting of memory problems, cognitive problems, vision problems, behavioral changes, personality changes, depression, and seizures.

31. The cell of any one of claims 26-30, wherein the cell comprises a nucleic acid sequence encoding the therapeutic molecule, wherein the nucleic acid sequence is operatively linked to a promoter responsive to amyloid beta-related pathology.

32. A modified cell that expresses and presents or secretes a therapeutic molecule, said modified cell comprises:
a. a nucleic acid sequence encoding a therapeutic molecule, the therapeutic molecule cleaves amyloid beta, binds to amyloid beta, enhances amyloid proteolysis, enhances amyloid proteolysis, enhances microglial phagocytosis of amyloid beta, or a combination thereof; and
b. a promoter selected from a CD9 gene promoter, a LGALS3 gene promoter, an HLA-DRB gene promoter, a TREM2 gene promoter, or a CD11c gene promoter, the promoter is operatively linked to the nucleic acid sequence encoding the therapeutic molecule.

33. A modified cell that expresses and presents or secretes a therapeutic molecule, said modified cell comprises:
a. a nucleic acid sequence encoding a therapeutic molecule, the therapeutic molecule cleaves amyloid beta, binds to amyloid beta, enhances amyloid proteolysis, enhances amyloid proteolysis, enhances microglial phagocytosis of amyloid beta, or a combination thereof; and
b. a promoter selected from a DCSTAMP gene promoter, a CD9 gene promoter, a CD44 gene promoter, an LGALS3 gene promoter, an SPP1 gene promoter, a GPNMB gene promoter, an HLA-DRB gene promoter, an LPL gene promoter, a LIPA gene promoter, a FABP3 gene promoter, an MS4A6A gene promoter, a CXCR4 gene promoter, a CHI3L1 gene promoter, an OLR1 gene promoter promoter, a CD36 gene promoter, a SLAMF8 gene promoter, a TREM2 gene promoter, an MSR1 gene promoter, a B2M gene promoter, an ITGAX gene promoter, and a MITF gene promoter; the promoter is operatively linked to the nucleic acid sequence encoding the therapeutic molecule.

34. A modified cell that expresses and presents or secretes a therapeutic molecule comprising membrane-bound neprilysin or secreted neprilysin, said modified cell comprising:
a. a nucleic acid sequence encoding the therapeutic molecule; and
b. a promoter operatively linked to the nucleic acid sequence encoding the therapeutic molecule, the promoter is configured to activate transcription of the therapeutic molecule upon (i) proximity of the cell to β-amyloid (Aβ) peptide plaques, soluble Aβ monomers, insoluble Aβ monomers, Aβ oligomers, pyroglutamate Aβ, protofibrils, or fibrils comprising Aβ of or a fragment thereof; or (ii) contact of the cell with β-amyloid (Aβ) peptide plaques, soluble Aβ monomers, insoluble Aβ monomers, Aβ oligomers, pyroglutamate Aβ, protofibrils, or fibrils comprising Aβ or a fragment thereof.

35. A modified cell that expresses and presents or secretes a therapeutic molecule comprising membrane-bound neprilysin, secreted neprilysin, TREM2, or insulin degrading enzyme, said modified cell comprising:
a. a nucleic acid sequence encoding the therapeutic molecule; and
b. a promoter operatively linked to the nucleic acid sequence encoding the therapeutic molecule, the promoter is configured to activate transcription of the therapeutic molecule upon (i) proximity of the cell to β-amyloid (Aβ) peptide plaques, soluble Aβ monomers, insoluble Aβ monomers, Aβ oligomers, pyroglutamate Aβ, protofibrils, or fibrils comprising Aβ of or a fragment thereof; or (ii) contact of the cell with β-amyloid (Aβ) peptide plaques, soluble Aβ monomers, insoluble Aβ monomers, Aβ oligomers, pyroglutamate Aβ, protofibrils, or fibrils comprising Aβ or a fragment thereof.

36. A microglia-like (MGL) cell that expresses and presents or secretes a therapeutic molecule that cleaves amyloid beta, binds to amyloid beta, enhances amyloid proteolysis, enhances enhances microglial phagocytosis of amyloid beta, or a combination thereof, said cell comprising:
a. a nucleic acid sequence encoding the therapeutic molecule; and
b. a promoter operatively linked to the nucleic acid sequence encoding the therapeutic molecule, the promoter is configured to activate transcription of the therapeutic molecule upon (i) proximity of the cell to β-amyloid (Aβ) peptide plaques, soluble Aβ monomers, insoluble Aβ monomers, Aβ oligomers, pyroglutamate Aβ, protofibrils, or fibrils comprising Aβ of or a fragment thereof; or (ii) contact of the cell with β-amyloid (Aβ) peptide plaques, soluble Aβ monomers, insoluble Aβ monomers, Aβ oligomers, pyroglutamate Aβ, protofibrils, or fibrils comprising Aβ or a fragment thereof.

37. The cell of any one of claims 31-36 wherein the promoter is configured to activate transcription of the therapeutic molecule upon (i) proximity of the cell to β-amyloid (Aβ) peptide plaques, soluble Aβ monomers, insoluble Aβ monomers, Aβ oligomers, pyroglutamate Aβ, protofibrils, or fibrils comprising Aβ of or a fragment thereof; or (ii) contact of the cell with β-amyloid (Aβ) peptide plaques, soluble Aβ monomers, insoluble Aβ monomers, Aβ oligomers, pyroglutamate Aβ, protofibrils, or fibrils comprising Aβ or a fragment thereof.

38. The cell of claim 37, wherein the cell expresses P2RY12 and TREM2.

39. The cell of any one of claims 31-35, wherein the cell is a migratory cell or is produced from a lineage of cells that can differentiate into migratory cells.

40. The cell of any one of claims 31-35, wherein the cell is a pluripotent stem cell (PSC), an induced pluripotent stem cells (iPSC), a myeloid progenitor cell, an erythromyeloid progenitor, a hematopoietic stem cell, a hematopoietic progenitor cell, a lymphoid progenitor cell, a megakaryocyte-erythroid (mk-ery), a cord-blood stem cell, or an embryonic stem cell.

41. The cell of any one of claims 31-35, wherein the cell is a monocyte.

42. The cell of any one of claims 31-35, wherein the cell is a bone marrow-derived hematopoietic precursor cell.

43. The cell of any one of claims 31-35, wherein the cell is a neural stem cell.

44. The cell of any one of claims 31-35, wherein the cell is an iPSC-derived microglial cell.

45. The cell of any one of claims 31-35, wherein the cell is an iPSC-derived hematopoietic precursor cell.

46. The cell of any one of claims 31-35, wherein the cell is a hematopoietic precursor cell.

47. The cell of any one of claims 31-35, wherein the cell is a microglia-like (MGL) cell.

48. The cell of claim 47, wherein the cell is a human induced microglia-like cell (hiMGL).

49. The cell of claim 47, wherein the MGL cell is a human induced pluripotent stem-cell-derived MGL cell.

50. The cell of any one of claims 31-36, wherein the cell is a microglia-like (MGL) cell capable of phagocytosing human synaptosomes.

51. The cell of any one of claims 31-36, wherein the cell is a microglia-like (MGL) cell capable of phagocytosing amyloid Aβ fibers.

52. The cell of any one of claims 31-36, wherein the cell is a microglia-like (MGL) cell capable of migrating to an injury site.

53. The cell of any one of 31-52, wherein the therapeutic protein is a membrane bound protein.

54. The cell of any one of claims 31-52, wherein the therapeutic protein is a secreted protein.

55. The cell of any one of claims 31-52, wherein the therapeutic protein is a metalloprotease.

56. The cell of any one of claims 31-52, wherein the therapeutic protein comprises neprilysin activity.

57. The cell of claim 31-52, wherein therapeutic molecule comprises membrane-bound neprilysin or secreted neprilysin.

58. The cell of any one of claims 31-52, wherein the therapeutic molecule comprises a peptide according to SEQ ID NO: 1, SEQ ID NO: 2, or SEQ ID NO: 3.

59. The cell of any one of claims 31-33 or 35-36, wherein the therapeutic molecule comprises TREM2, APOE, LRP1, or insulin degrading enzyme, endothelin-converting enzyme, plasminogen activator, angiotensin-converting enzyme, or a matrix metalloproteinase.

60. The cell of any one of claims 31-59, wherein the promoter is selected from a CD9 gene promoter, a LGALS3 gene promoter, a TREM2 gene promoter, an HLA-DRB gene promoter, or a CD11c gene promoter.

61. The cell of any one of claims 31 or 34-36, wherein the promoter is selected from: a DCSTAMP gene promoter, a CD9 gene promoter, a CD44 gene promoter, an LGALS3 gene promoter, an SPP1 gene promoter, a GPNMB gene promoter, an HLA-DRB gene promoter, an LPL gene promoter, a LIPA gene promoter, a FABP3 gene promoter, an MS4A6A gene promoter, a CXCR4 gene promoter, a CHI3L1 gene promoter, an OLR1 gene promoter promoter, a CD36 gene promoter, a SLAMF8 gene promoter, a TREM2 gene promoter, an MSR1 gene promoter, a B2M gene promoter, an ITGAX gene promoter, and a MITF gene promoter.

62. The cell of any one of claims 31-61, wherein the nucleic acid sequence is inserted into the genome of the cell.

63. The cell of claim 62, wherein the nucleic acid sequence is inserted downstream of a locus controlled by the promoter such that the nucleic acid sequence is in-frame with a coding sequence in an exon of the locus.

64. The cell of claim 62, wherein the nucleic acid sequence is inserted within the locus controlled by the promoter such that the nucleic acid sequence is joined, inframe, with at least part of the coding sequence of the locus.

65. The cell of any one of claims 63-64, wherein a first polynucleotide encoding a protease cleavage site, a ribosomal skipping sequence, or a self-cleaving peptide is inserted between the coding sequence of the exon and the nucleic acid sequence encoding the therapeutic protein.

66. The cell of claim 65, wherein the self-cleaving peptide is P2A.

67. The cell of claim 65, wherein a second polynucleotide encoding a secreted peptide signal sequence is inserted at the 5′ end of the nucleic acid sequence encoding the therapeutic protein.

68. The cell of any one of claims 26-35, wherein the therapeutic protein affects one or more amyloid-beta (Aβ)-related pathologies.

69. The cell of claim 68, wherein the Aβ-related pathology comprises β-amyloid (Aβ) peptide plaques, soluble Aβ monomers, insoluble Aβ monomers, Aβ oligomers, pyroglutamate Aβ, protofibrils, or fibrils comprising Aβ of varying lengths.

70. The cell of any one of claims 68-69, wherein the therapeutic molecule reduces the amount of Aβ peptide in Aβ peptide plaques in a brain of an individual.

71. The cell of any one of claims 68-69, wherein the therapeutic molecule reduces the size or number of soluble Aβ monomers, insoluble Aβ monomers, Aβ oligomers, pyroglutamate Aβ, protofibrils, or fibrils comprising Aβ of varying lengths; enhances amyloid proteolysis; enhances microglial phagocytosis of amyloid beta, or a combination thereof.

72. A composition comprising the cell of any one of claims 1-71.

73. A kit comprising the cell of any one of claims 1-71 or the composition of claim 72.

74. A composition for use in a method of treating a disease or condition associated with amyloid beta-related pathology or ameliorating symptoms or pathological processes associated with a disease or condition associated with amyloid beta-related pathology, said composition comprising a modified cell of any one of claims 1-71.

75. A method of treating an individual having a disease or condition associated with amyloid beta-related pathology, said method comprising administering to at least one target brain region in a brain of the individual the cell of any one of claims 1-71 or the composition of claim 72.

76. A method of reducing:
i) an amount of Aβ peptide in Aβ peptide plaques; and/or
ii) a size or number of soluble Aβ monomers, insoluble Aβ monomers, Aβ oligomers, pyroglutamate Aβ, protofibrils, or fibrils comprising Aβ of varying lengths;
in a brain of an individual;
comprising administering to at least one target brain region in a brain of the individual the cell of any one of claims 1-71, or the composition of claim 72, to the individual.

77. A method of reducing neuronal or synaptic loss in a subject in need thereof, the subject having an amyloid beta-related pathology comprising Aβ plaques in brain tissue, said method comprising administering to at least one target brain region in a brain of the individual the cell of any one of claims 1-71, or the composition of claim 72, to the individual.

78. A method of preventing or attenuating onset of a neurodegenerative disorder or condition associated with amyloid beta pathology, said method comprising administering to at least one target brain region in a brain of the individual the cell of any one of claims 1-71 or the composition of claim 72.

79. The method of any one of claims 75-78, wherein the at least one target brain region comprises a cerebral cortex or subregion thereof, a hippocampus or subregion thereof, a cerebral ventricle, a basal ganglia or basal ganglia nucleus thereof, an entorhinal cortex, a medial prefrontal cortex or subregion thereof, an anterior cingulate cortex or subregion thereof, a primary sensory cortex or sensory association cortex, a fornix, a septum, or a combination thereof.

80. The method of any one of claims 75-78, wherein the amyloid beta-related pathology is Alzheimer's disease, Parkinson's disease, Huntington's disease, or amyotrophic lateral sclerosis (ALS).

81. The method of any one of claims 75-79, wherein the cell is administered prior to onset of Aβ-related pathology.

82. The method of any one of claims 75-79, wherein the cell is administered prior to a presence of β-amyloid (Aβ) peptide plaques, soluble Aβ monomers, insoluble Aβ monomers, Aβ oligomers, pyroglutamate Aβ, protofibrils, or fibrils comprising Aβ or a fragment thereof in the individual.

83. The method of any one of claims 75-79, wherein the cell is administered after detection of a presence of β-amyloid (Aβ) peptide plaques, soluble Aβ monomers, insoluble Aβ monomers, Aβ oligomers, pyroglutamate Aβ, protofibrils, or fibrils comprising Aβ or a fragment thereof in the individual.

84. The method of any one of claims 75-79, wherein the cell is administered via stereotactic injection into the brain of the individual.

85. The method of any one of claims 75-84, wherein an amount of Aβ peptide or Aβ peptide plaques is determined using amyloid beta PET imaging, MRI, a histological method, an immunoblotting method, an amyloid beta staining method, or a combination thereof.

86. Use of the cell of any one of claims 1-71, the composition of claim 72, or the kit of claim 73, in:
i) reducing the amount of Aβ peptide in Aβ peptide plaques;
ii) reducing the size or number of soluble Aβ monomers, insoluble Aβ monomers, Aβ oligomers, pyroglutamate Aβ, protofibrils, or fibrils comprising Aβ of varying lengths Aβ peptide deposition.

87. The use according to claim 86, wherein use is for a method comprising transplanting hiMGLs into a brain tissue of an individual.

88. Use of the cell of any one of claims 1-71, the composition of claim 72, or the kit of claim 73, in treating a disease associated with amyloid beta-related pathology, or ameliorating symptoms or pathological processes associated with amyloid beta-related pathology.


SEQUENCE LISTING (16 sequences)

SEQ ID NO: 1 - protein, 750 aa, Homo sapiens
MGKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 2 - protein, 699 aa, Homo sapiens
YDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 3 - protein, 728 aa, Homo sapiens
MGAASGRRGPGLLLPLPLLLLLPPQPALAYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 4 - DNA, 2307 bp, Homo sapiens
  source 1..2307: mol_type = genomic DNA
gctactaacttcagcctgctgaagcaggctggagacgtggaggagaaccctggacctatgggcaagtcagaaagtcagatggatataactgatatcaacactccaaagccaaagaagaaacagcgatggactccactggagatcagcctctcggtccttgtcctgctcctcaccatcatagctgtgacaatgatcgcactctatgcaacctacgatgatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgacattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacctcgagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttgg

SEQ ID NO: 5 - DNA, 2241 bp, Homo sapiens
  source 1..2241: mol_type = genomic DNA
gctactaacttcagcctgctgaagcaggctggagacgtggaggagaaccctggacctatgggcgccgctagcggccggcgcggccccgggctgctcctgcccctgccactgctgctgctcctgccccctcagcccgccctggcctacgatgatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgacattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacctcgagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttgg

SEQ ID NO: 6 - DNA, 1000 bp, synthetic construct
  source 1..1000: mol_type = other DNA
tagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgtacataaaagaatttttttgtctttaaatagatacaaatgtctatcaactttaatcaagttgtaacttatattgaagacaatttgatacataataaaaaattatgacaatgtcctggactggtttttgttttgtttaattattttgagagctatttggtttgcaaatgtctttcaactcctattaactccacctgcatttttttttttttttaagacggagtttcgctcttgttgcccaggctggagtgcaatggcccgatcttggctcaccacagcctccacctcctgggttcaaacaattctcctgcctcagcttcctgagtagctgggattacaggcatgcaccaccacacccggctaattttgtatttttagtagagacggggtttctccgtgttggtcaggctggtctcgaactcccaacctcagttgatccgcccgcctcagcctcccaaagtgctgggattacaggcgtgagccaccgtgcccggccccacctgcatttatatgagaagtagattgggaggttgctcaccccagtttactaatgaagaaatctagatgtggagagattaaaggattctgatccaaagtcacacaggtgtcaacagaggagtcagcaccagcacctggtcctcagacgctcattcagcagtggggtatatcttggggatat

SEQ ID NO: 7 - DNA, 1000 bp, synthetic construct
  source 1..1000: mol_type = other DNA
tcccccacccccgcctgcctctgccctcttgccagactgcccccaaagagagaatcgcctcctttcttagtggttatttgcacccttcaccggcatcttaagaaaggcatgaagcacctttcagcaaaatatacgacaataccttcttttttttctttttttctttttttgagacgaagtttcactcttgtcgcccaggctggagtgcagtggtgtgatctcgactcactgcaacctccgcctctctgggttcaagcaattatccagcctcagcctcccgagtagctgggactacaggcgtgtgccaccatgcccagctgattatttttagtagagatggggtttcaccatgttggtcaggctggtctcgaactcctgacctcaggtaatctgcctgccttggcctcccaaagtgctgggattacaggcgtgagccactgtgcccagccaagatataacaatacgttttaaaatagaatattaacattttgggaggccgaggcaggcggatcgcaccacagcactctagcctaagcaacagagcaagactccatctcaaaagaaaaaaaaaagagtattaaataacatacacacaaagacaaatgcaaaagtactaacctgtatggactaggactataggaaaacctggaatttgagcattaaatttggctctgagcttactttcccaccctgaaagttctccaaggaaaatgcttgtgcagctgactgccaacccccccagattcctcacgtcccgcacccttctcattatctggcctgcatctctcctgcttcagcaccttctgactcgtttggcctcctggatgccattctccttgtcatttttcttggactgggtgacccatgtctctccctttccctcagccttccttcagatcaaaccaccctgatcctcatgtttcttcctatctcctagatatttggcatgatcttcagtatgatcttgtgctgtgctatccgcaggaaccgcgagatggtc

SEQ ID NO: 8 - RNA, 23 nt, synthetic construct
  source 1..23: mol_type = other RNA
gctgactctagaccatctcgcgg

SEQ ID NO: 9 - protein, 997 aa, synthetic construct
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMVATNFSLLKQAGDVEENPGPMGKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 10 - protein, 928 aa, synthetic construct
MMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMVATNFSLLKQAGDVEENPGPMGKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 11 - protein, 975 aa, synthetic construct
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMVATNFSLLKQAGDVEENPGPMGAASGRRGPGLLLPLPLLLLLPPQPALAYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 12 - protein, 906 aa, synthetic construct
MMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMVATNFSLLKQAGDVEENPGPMGAASGRRGPGLLLPLPLLLLLPPQPALAYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 13 - protein, 228 aa, Homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 14 - protein, 159 aa, Homo sapiens
MMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 15 - protein, 19 aa, Homo sapiens
ATNFSLLKQAGDVEENPGP

SEQ ID NO: 16 - protein, 29 aa, Homo sapiens
MGAASGRRGPGLLLPLPLLLLLPPQPALA